(19) United States
(12) Patent Application Publication
(10) Pub. No.: US 2022/0267356 A1
(43) Pub. Date: Aug. 25, 2022

(54) ARGINASE INHIBITORS AND METHODS OF USE THEREOF

(71) Applicant: AstraZeneca AB, Södertälje (SE)

(72) Inventors: Scott Nathan MLYNARSKI, Wilmington, DE (US); Jason SHIELDS, Wilmington, DE (US); Sameer KAWATKAR, Wilmington, DE (US); Qing YE, Wilmington, DE (US); Haixia WANG, Wilmington, DE (US); Xiaolan ZHENG, Wilmington, DE (US); Ray FINLAY, Cambridge (GB); Iain SIMPSON, Cambridge (GB)

(21) Appl. No.: 17/628,921

(22) PCT Filed: Jul. 22, 2020
(86) PCT No.: PCT/IB2020/056899

§ 371 Date: Jan. 21, 2022

Related U.S. Application Data
(60) Provisional application No. 62/877,407, filed on Jul. 23, 2019.

(51) Int. Cl. C07F 5/02

(52) U.S. Cl. CPC C07F 5/025

(57) ABSTRACT

Disclosed are compounds of formula (I), or pharmaceutically acceptable salts thereof, pharmaceutical compositions comprising the same, and methods of treating cancer or a respiratory inflammatory disease using the same:

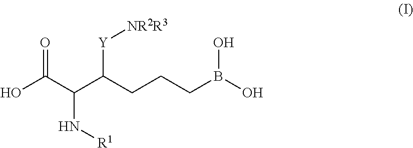

wherein
R¹ is selected from hydrogen, —CH₃ and —(C═O)CH(R^{1a})NH₂;
R^{1a} is C₁-C₄ alkyl;
Y is —(CH₂)ₙ— or —(C═O)—;
n is an integer selected from 1 and 2;
R² is selected from hydrogen, —CH₃ and —(C═X)R⁴ and R³ is hydrogen or —CH₃; or
R² and R³, together with the nitrogen to which they are attached, are linked to form a 6-membered heterocyclic ring;
X is NH or O;
R⁴ is —CH₃ or —[CH(R^{4a})]ₘNH₂;
m is an integer selected from 0 or 1; and
R^{4a} is hydrogen or C₁-C₆ alkyl.

### BACKGROUND

Arginase is a manganese metalloenzyme that catalyzes the conversion of L-arginine to urea and L-ornithine. Two isoforms exist: Arginase 1 is a cytosolic enzyme predominantly found in hepatocytes where it plays a critical role in removing ammonia through urea synthesis, and Arginase 2, a mitochondrial enzyme highly expressed in kidney involved in production of ornithine, a precursor for polyamines and prolines important for cell proliferation and collagen production, respectively.

Although L-arginine is not an essential amino acid as it can be provided through protein turnover in healthy adults, increased expression and secretion of arginases results in reduced L-arginine levels in various physiologic and pathologic conditions (e.g., pregnancy, auto-immune diseases, cancer). Immune cells, in particular, are sensitive to reduced L-arginine levels. T-cells, when faced with a low L-arginine microenvironment, reduce their proliferation rate and lower the expression of CD3ξ chain, IFNγ, and lytic enzymes resulting in impaired T-cell responsiveness. Dendritic cells respond to low L-arginine conditions by reducing their ability to present antigens, and natural killer cells reduce both proliferation and expression of lytic enzymes.

Tumors use multiple immune suppressive mechanisms to evade the immune system. One of these is the reduction of L-arginine through increased levels of circulating arginase, increased expression and secretion of arginase by tumor cells, and recruitment of arginase expressing and secreting myeloid derived suppressor cells. Together, these lead to a reduction of L-arginine in the tumor microenvironment and an immune-suppressive phenotype. Pharmacologic inhibition of arginase activity has been shown to reverse the low L-arginine induced immune suppression in animal models. As such, there is a need for potent and selective arginase inhibitors to reverse immune suppression and re-activate anti-cancer immunity in patients, either as single agent, or in combination with therapies reversing additional immune-suppressive mechanisms.

### SUMMARY

In some embodiments, disclosed are compounds of formula (I), or a pharmaceutically acceptable salt thereof:

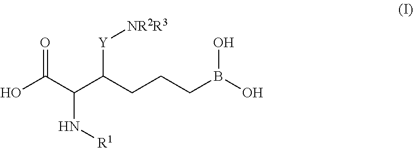

wherein

R¹ is selected from hydrogen, —CH₃ and —(C═O)CH(R^{1a})NH₂;

R^{1a} is C₁-C₄ alkyl;

Y is —(CH₂)ₙ— or —(C═O)—;

n is an integer selected from 1 and 2;

R² is selected from hydrogen, —CH₃ and —(C═X)R⁴ and R³ is hydrogen or —CH₃; or

R² and R³, together with the nitrogen to which they are attached, are linked to form a nitrogen-containing 6-membered heterocyclic ring;

X is NH or O;

R⁴ is —CH₃ or —[CH(R^{4a})]ₘNH₂;

m is an integer selected from 0 or 1; and

R^{4a} is hydrogen or C₁-C₄ alkyl.

In some embodiments, disclosed is a compound of formula (II), or a pharmaceutically acceptable salt thereof:

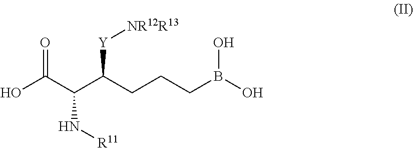

wherein

R¹¹ is selected from hydrogen, —CH₃ and

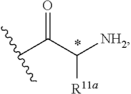

wherein * indicates (S) stereochemistry;

Y¹ is —(CH₂)ₚ— or —(C═O)—;

p is an integer selected from 1 and 2;

R^{11a} is C₁-C₄ alkyl;

R¹² is selected from hydrogen, —CH₃ and —(C═X¹)R¹⁴ and R¹³ is hydrogen or —CH₃; or

R¹² and R¹³, together with the nitrogen to which they are attached, are linked to form a nitrogen-containing 6-membered heterocyclic ring;

X¹ is NH or O;

R¹⁴ is —CH₃ or

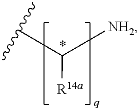

wherein * indicates (S) stereochemistry;

R^{14a} is C₁-C₄ alkyl; and

q is an integer selected from 0 and 1.

In some embodiments, disclosed is a compound of formula (III), or a pharmaceutically acceptable salt thereof:

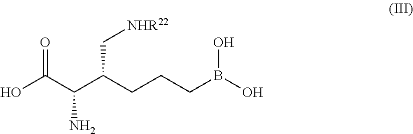

wherein

R²² is hydrogen or

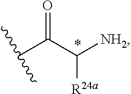

wherein * indicates (S) stereochemistry; and

R^{24a} is C₁-C₄ alkyl.

In some embodiments, disclosed is a compound of formula (IV), or a pharmaceutically acceptable salt thereof:

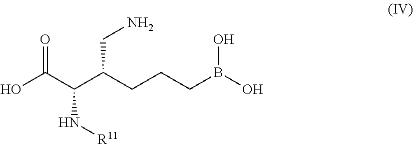

wherein

R¹¹ is

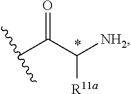

wherein * indicates (S) stereochemistry; and R^{11a} is C₁-C₄ alkyl.

In some embodiments, disclosed are the compounds of Table 1, or a pharmaceutically acceptable salt thereof.

In some embodiments, disclosed are pharmaceutical compositions comprising a compound of formula (I), (Ia), (Ib), (II), (III), (IV) or Table 1, or a pharmaceutically acceptable salt thereof, and a pharmaceutically acceptable carrier.

In some embodiments, disclosed are methods of treating cancer comprising administering a compound of formula (I), (Ia), (Ib), (II), (III), (IV) or Table 1, or a pharmaceutically acceptable salt thereof.

In some embodiments, disclosed are compounds of formula (I), (Ia), (Ib), (II), (III), (IV) or Table 1, or a pharmaceutically acceptable salt thereof, for treating cancer.

In some embodiments, disclosed is the use of a compound of (I), (Ia), (Ib), (II), (III), (IV) or Table 1, or a pharmaceutically acceptable salt thereof in the manufacture of a medicament for use in treating cancer.

In some embodiments, disclosed are pharmaceutical compositions comprising a compound of formula (I), (Ia), (Ib), (II), (III), (IV) or Table 1, or a pharmaceutically acceptable salt thereof, for use in treating cancer.

In some embodiments, disclosed are methods of treating a respiratory inflammatory disease comprising administering a compound of formula (I), (Ia), (Ib), (II), (III), (IV) or Table 1, or a pharmaceutically acceptable salt thereof.

In some embodiments, disclosed are compounds of formula (I), (Ia), (Ib), (II), (III), (IV) or Table 1, or a pharmaceutically acceptable salt thereof, for treating a respiratory inflammatory disease.

In some embodiments, disclosed is the use of a compound of (I), (Ia), (Ib), (II), (III), (IV) or Table 1, or a pharmaceutically acceptable salt thereof in the manufacture of a medicament for use in treating a respiratory inflammatory disease.

In some embodiments, disclosed are pharmaceutical compositions comprising a compound of formula (I), (Ia), (Ib), (II), (III), (IV) or Table 1, or a pharmaceutically acceptable salt thereof, for use in treating a respiratory inflammatory disease.

In some embodiments, the aforementioned respiratory inflammatory disease is chronic obstructive pulmonary disease (COPD) or asthma.

### DETAILED DESCRIPTION

In some embodiments, disclosed is a compound of formula (I), or a pharmaceutically acceptable salt thereof:

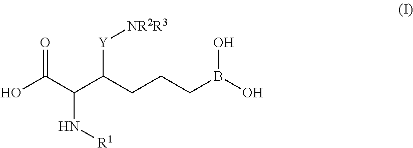

wherein

R¹ is selected from hydrogen, —CH₃ and —(C═O)CH(R^{1a})NH₂;

R^{1a} is C₁-C₄ alkyl;

Y is —(CH₂)ₙ— or —(C═O)—;

n is an integer selected from 1 and 2;

R² is selected from hydrogen, —CH₃ and —(C═X)R⁴ and R³ is hydrogen or —CH₃; or

R² and R³, together with the nitrogen to which they are attached, are linked to form a nitrogen-containing 6-membered heterocyclic ring;

X is NH or O;

R⁴ is —CH₃ or —[CH(R^{4a})]ₘNH₂;

m is an integer selected from 0 or 1; and

R^{4a} is C₁-C₄ alkyl.

In some embodiments in the compound of formula (I), R¹ is hydrogen, Y is —(CH₂)ₙ—, n is 1, R² is hydrogen and R³ is hydrogen.

In some embodiments in the compound of formula (I), R¹ is hydrogen, Y is —(CH₂)ₙ—, n is 1, R² are R³, together with the nitrogen to which they are attached, are linked to form a nitrogen-containing six-membered heterocyclic ring. In some embodiments, the nitrogen-containing six-membered heterocyclic ring is a morpholinyl ring. In some embodiments, the nitrogen-containing six-membered heterocyclic ring is a piperidinyl ring.

In some embodiments in the compound of formula (I), R¹ is hydrogen, Y is —(CH₂)ₙ—, n is 1, R² is —CH₃ and R³ is hydrogen.

In some embodiments in the compound of formula (I), R¹ is hydrogen, Y is —(CH₂)ₙ—, n is 1, R² is —CH₃ and R³ is —CH₃.

In some embodiments in the compound of formula (I), R¹ is hydrogen, Y is —(CH₂)ₙ—, n is 1, R² is —(C═X)R⁴, R³ is hydrogen, X is NH, R⁴ is [CH(R^{4a})]ₘNH₂ and m is 0.

In some embodiments in the compound of formula (I), R¹ is hydrogen, Y is —(CH₂)ₙ—, n is 1, R² is —(C═X)R⁴, R³ is hydrogen, X is O, R⁴ is [CH(R^{4a})]ₘNH₂ and m is 0.

In some embodiments in the compound of formula (I), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is C₃ alkyl (e.g., isopropyl).

In some embodiments in the compound of formula (I), R¹ is hydrogen, Y is —(CH₂)ₙ—, n is 1, R² is —(C═X)R⁴m R³ is hydrogen, X is O, R⁴ is —[CH(R^{4a})]ₘNH₂, m is 1 and R^{4a} is C₂ alkyl (e.g., ethyl).

In some embodiments in the compound of formula (I), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is C₁ alkyl (e.g., methyl).

In some embodiments in the compound of formula (I), R¹ is hydrogen, Y is —(CH₂)ₙ—, n is 1, R² is —(C═X)R⁴, R³ is hydrogen, X is O and R⁴ is —CH₃.

In some embodiments in the compound of formula (I), R¹ is —CH₃, Y is —(CH₂)ₙ—, n is 1, R² is hydrogen and R³ is hydrogen.

In some embodiments in the compound of formula (I), R¹ is —(C═O)CH(R^{1a})NH₂, R^{1a} is C₃ alkyl (e.g., isopropyl), R is —(CH₂)ₙ—, n is 1, R² is hydrogen and R³ is hydrogen.

In some embodiments in the compound of formula (I), R¹ is —(C═O)CH(R^{1a})NH₂, R^{1a} is C₁ alkyl (e.g., methyl), R is —(CH₂)ₙ—, n is 1, R² is hydrogen and R³ is hydrogen.

In some embodiments in the compound of formula (I), R¹ is hydrogen, Y is —(CH₂)ₙ—, n is 2, R² is hydrogen and R³ is hydrogen.

In some embodiments in the compound of formula (I), R¹ is hydrogen, Y is —(C═O), R² is hydrogen and R³ is hydrogen.

In some embodiments in the compound of formula (I), R¹ is hydrogen, Y is —(C═O), R² is —CH₃ and R³ is hydrogen.

In some embodiments in the compound of formula (I), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is C₄ alkyl (e.g., isobutyl).

In some embodiments in the compound of formula (I), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is C₄ alkyl (e.g., tert-butyl).

In some embodiments in the compound of formula (I), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is hydrogen.

In some embodiments the compound of formula (I), or a pharmaceutically acceptable salt thereof, is a compound of formula (Ia):

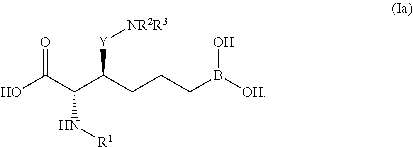

In some embodiments in the compound of formula (Ia), R¹ is hydrogen, Y is —(CH₂)ₙ—, n is 1, R² is hydrogen and R³ is hydrogen.

In some embodiments in the compound of formula (Ia), R¹ is hydrogen, Y is —(CH₂)ₙ—, n is 1, R² and R³, together with the nitrogen to which they are attached, are linked to form a nitrogen-containing six-membered heterocyclic ring. In some embodiments, the nitrogen-containing six-membered heterocyclic ring is a morpholinyl ring. In some embodiments, the nitrogen-containing six-membered heterocyclic ring is a piperidinyl ring.

In some embodiments in the compound of formula (Ia), R¹ is hydrogen, Y is —(CH₂)ₙ—, n is 1, R² is —CH₃ and R³ is hydrogen.

In some embodiments in the compound of formula (Ia), R¹ is hydrogen, Y is —(CH₂)ₙ—, n is 1, R² is —CH₃ and R³ is —CH₃.

In some embodiments in the compound of formula (Ia), R¹ is hydrogen, Y is —(CH₂)ₙ—, n is 1, R² is —(C═X)R⁴, R³ is hydrogen, X is NH, R⁴ is [CH(R^{4a})]ₘNH₂ and m is 0.

In some embodiments in the compound of formula (Ia), R¹ is hydrogen, Y is —(CH₂)ₙ—, n is 1, R² is —(C═X)R⁴, R³ is hydrogen, X is O, R⁴ is [CH(R^{4a})]ₘNH₂ and m is 0.

In some embodiments in the compound of formula (Ia), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is C₃ alkyl (e.g., isopropyl).

In some embodiments in the compound of formula (Ia), R¹ is hydrogen, Y is —(CH₂)ₙ—, n is 1, R² is —(C═X)R⁴m R³ is hydrogen, X is O, R⁴ is —[CH(R^{4a})]ₘNH₂, m is 1 and R^{4a} is C₂ alkyl (e.g., ethyl).

In some embodiments in the compound of formula (Ia), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is C₁ alkyl (e.g., methyl).

In some embodiments in the compound of formula (Ia), R¹ is hydrogen, Y is —(CH₂)ₙ—, n is 1, R² is —(C═X)R⁴, R³ is hydrogen, X is O and R⁴ is —CH₃.

In some embodiments in the compound of formula (Ia), R¹ is —CH₃, Y is —(CH₂)ₙ—, n is 1, R² is hydrogen and R³ is hydrogen.

In some embodiments in the compound of formula (Ia), R¹ is —(C═O)CH(R^{1a})NH₂, R^{1a} is C₃ alkyl (e.g., isopropyl), R is —(CH₂)ₙ—, n is 1, R² is hydrogen and R³ is hydrogen.

In some embodiments in the compound of formula (Ia), R¹ is —(C═O)CH(R^{1a})NH₂, R^{1a} is C₁ alkyl (e.g., methyl), R is —(CH₂)ₙ—, n is 1, R² is hydrogen and R³ is hydrogen.

In some embodiments in the compound of formula (Ia), R¹ is hydrogen, Y is —(CH₂)ₙ—, n is 2, R² is hydrogen and R³ is hydrogen.

In some embodiments in the compound of formula (Ia), R¹ is hydrogen, Y is —(C═O), R² and R³ are hydrogen.

In some embodiments in the compound of formula (Ia), R¹ is hydrogen, Y is —(C═O), R² is —CH₃ and R³ is hydrogen.

In some embodiments the compound of formula (I), or a pharmaceutically acceptable salt thereof, is a compound of formula (Ib):

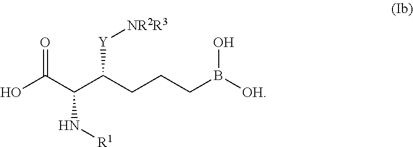

In some embodiments in the compound of formula (Ib), R¹ is hydrogen, Y is —(CH₂)ₙ—, n is 1, R² is hydrogen and R³ is hydrogen.

In some embodiments in the compound of formula (Ib), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is C₃ alkyl (e.g., isopropyl).

In some embodiments in the compound of formula (Ib), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is C₁ alkyl (e.g., methyl).

In some embodiments in the compound of formula (Ib), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is C₂ alkyl (e.g., ethyl).

In some embodiments in the compound of formula (Ib), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is C₄ alkyl (e.g., isobutyl or tert-butyl).

In some embodiments in the compound of formula (Ib), R¹ is hydrogen, Y is —(CH₂)ₙ—, n is 1, R² is —(C═X)R⁴, R³ is hydrogen, X is O, R⁴ is —[CH(R^{4a})]ₘNH₂, m is 1 and R^{4a} is hydrogen.

In some embodiments disclosed is a compound of formula (II), or a pharmaceutically acceptable salt thereof:

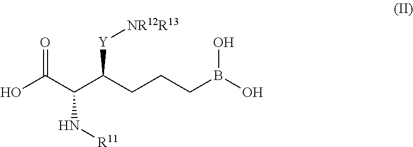

wherein

R¹¹ is selected from hydrogen, —CH₃ and

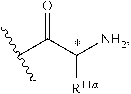

wherein * indicates (S) stereochemistry;

Y¹ is —(CH₂)ₚ— or —(C═O);

p is an integer selected from 1 and 2;

R^{11a} is C₁-C₄ alkyl;

R¹² is selected from hydrogen, —CH₃ and —(C═X¹)R¹⁴ and R¹³ hydrogen or —CH₃; or

R¹² and R¹³, together with the nitrogen to which they are attached, are linked to form a 6-membered heterocyclic ring;

X¹ is NH or O;

R¹⁴ is —CH₃ or

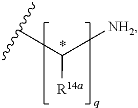

wherein * indicates (S) stereochemistry;

R^{14a} is C₁-C₄ alkyl; and

q is an integer selected from 0 and 1.

In some embodiments in the compound of formula (II), R¹¹ is hydrogen, Y¹ is —(CH₂)ₚ—, p is 1, R¹² is hydrogen and R¹³ is hydrogen.

In some embodiments in the compound of formula (II), R¹¹ is hydrogen, Y¹ is —(CH₂)ₚ—, p is 1 and R¹² and R¹³, together with the nitrogen to which they are attached, are linked to form a 6-membered nitrogen-containing heterocyclic ring. In some embodiments, the nitrogen-containing six-membered heterocyclic ring is a morpholinyl ring. In some embodiments, the nitrogen-containing six-membered heterocyclic ring is a piperadinyl ring.

In some embodiments in the compound of formula (II), R¹¹ is hydrogen, Y¹ is —(CH₂)ₚ—, p is 1, R¹² is —CH₃ and R¹³ is hydrogen.

In some embodiments in the compound of formula (II), R¹¹ is hydrogen, Y¹ is —(CH₂)ₚ—, p is 1, R¹² is —CH₃ and R¹³ is —CH₃.

In some embodiments in the compound of formula (II), R¹¹ is hydrogen, Y¹ is —(CH₂)ₚ—, p is 1, R¹² is —(C═X¹)R¹⁴, R¹³ is hydrogen, X¹ is NH, R¹⁴ is

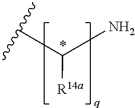

and q is 0.

In some embodiments in the compound of formula (II), R¹¹ is hydrogen, Y¹ is —(CH₂)ₚ—, p is 1, R¹² is —(C═X¹)R¹⁴, R¹³ is hydrogen, X¹ is 0, R¹⁴ is

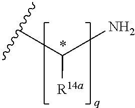

and q is 0.

In some embodiments in the compound of formula (II), R¹¹ is hydrogen, Y¹ is —(CH₂)ₚ—, p NH₂ is 1, R¹² is —(C═X¹)R¹⁴, R¹³ is hydrogen, X¹ is 0, R¹⁴ is

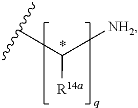

q is 1 and R^{14a} is C₃ alkyl (e.g., isopropyl).

In some embodiments in the compound of formula (II), R¹¹ is hydrogen, Y¹ is —(CH₂)ₚ—, p is 1, R¹² is —(C═X¹)R¹⁴, R¹³ is hydrogen, X¹ is 0, R¹⁴ is

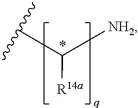

q is 1 and R^{14a} is C₂ alkyl (e.g., ethyl).

In some embodiments in the compound of formula (II), R¹¹ is hydrogen, Y¹ is —(CH₂)ₚ—, p is 1, R¹² is —(C═X¹)R¹⁴, R¹³ is hydrogen, X¹ is 0, R¹⁴ is

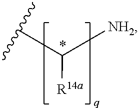

q is 1 and R^{14a} is C₁ alkyl (e.g., methyl).

In some embodiments in the compound of formula (II), R¹¹ is hydrogen, Y¹ is —(CH₂)ₚ—, p is 1, R¹² is —(C═X¹)R¹⁴, R¹³ is hydrogen, X¹ is O and R¹⁴ is —CH₃.

In some embodiments in the compound of formula (II), R¹¹ is —CH₃, Y¹ is —(CH₂)ₚ—, p is 1, R¹² is hydrogen and R¹³ is hydrogen.

In some embodiments in the compound of formula (II), R¹¹ is

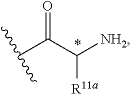

R^{11a} is C₃ alkyl (e.g., isopropyl), Y¹ is —(CH₂)ₚ—, p is 1, R¹² is hydrogen and R¹³ is hydrogen.

In some embodiments in the compound of formula (II), R¹¹ is

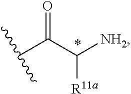

R^{11a} is C₁ alkyl (e.g., methyl), Y¹ is —(CH₂)ₚ—, p is 1, R¹² is hydrogen and R¹³ is hydrogen.

In some embodiments in the compound of formula (II), R¹¹ is hydrogen, Y¹ is —(CH₂)ₚ—, p is 2, R¹² is hydrogen and R¹³ is hydrogen.

In some embodiments in the compound of formula (II), R¹¹ is hydrogen, Y¹ is —(C═O), R¹² is hydrogen and R¹³ is hydrogen.

In some embodiments in the compound of formula (II), R¹¹ is hydrogen, Y¹ is —(C═O), R¹² is —CH₃ and R¹³ is hydrogen.

In some embodiments disclosed is a compound of formula (III), or a pharmaceutically acceptable salt thereof:

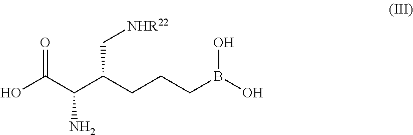

wherein

R²² is hydrogen or

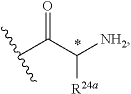

wherein * indicates (S) stereochemistry; and

R^{24a} is C₁-C₄ alkyl.

In some embodiments in the compound of formula (III), R²² is hydrogen.

In some embodiments in the compound of formula (III), R²² is

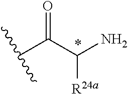

and R^{24a} is C₃ alkyl (e.g., isopropyl).

In some embodiments in the compound of formula (III), R²² is

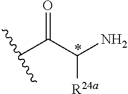

and R^{24a} is C₁ alkyl (e.g., methyl).

In some embodiments in the compound of formula (III), R²² is

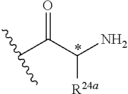

and R^{24a} is C₂ alkyl (e.g., ethyl).

In some embodiments in the compound of formula (III), R²² is

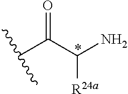

and R^{24a} is C₄ alkyl (e.g., isobutyl or tert-butyl).

In some embodiments, disclosed is a compound of formula (IV), or a pharmaceutically acceptable salt thereof:

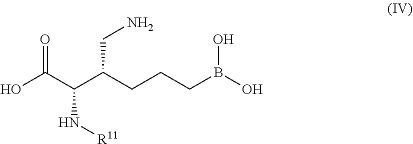

wherein

R¹¹ is

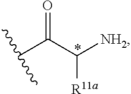

wherein * indicates (S) stereochemistry; and R^{11a} is C₁-C₄ alkyl.

In some embodiments, disclosed is a compound of Table 1, or a pharmaceutically acceptable salt thereof:

TABLE 1

| Example | Compound | Name |
|---|---|---|
| 1 | 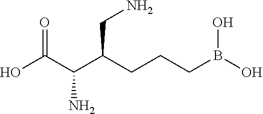 [SMILES: NC[C@@H](CCCB(O)O)[C@H](N)C(=O)O] | (2S,3R)-2-amino-3-(aminomethyl)-6- boronohexanoic acid |
| 2 | 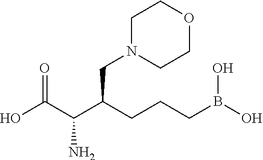 [SMILES: N[C@H](C(=O)O)[C@H](CCCB(O)O)CN1CCOCC1] | (2S,3R)-2-amino-6-borono-3- (morpholinomethyl)hexanoic acid |
| 3 | 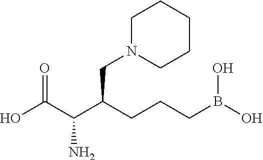 [SMILES: N[C@H](C(=O)O)[C@H](CCCB(O)O)CN1CCCCC1] | (2S,3R)-2-amino-6-borono-3-(piperidin-1- ylmethyl)hexanoic acid |
| 4 | 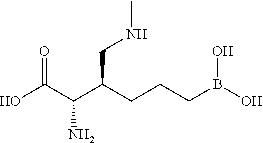 | (2S,3R)-2-amino-6-borono-3- ((methylamino)methyl)hexanoic acid |
| 5 | 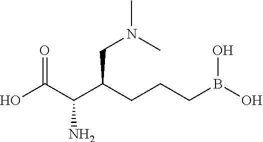 [SMILES: CN(C)C[C@@H](CCCB(O)O)[C@H](N)C(=O)O] | (2S,3R)-2-amino-6-borono-3- ((dimethylamino)methyl)hexanoic acid |
| 6 | 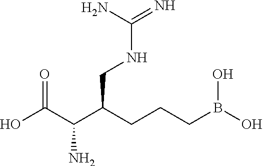 [SMILES: N=C(N)NC[C@@H](CCCB(O)O)[C@H](N)C(=O)O] | (2S,3R)-2-amino-6-borono-3- (guanidinomethyl)hexanoic acid |
| 7 | 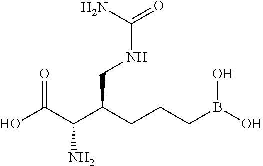 [SMILES: NC(=O)NC[C@@H](CCCB(O)O)[C@H](N)C(=O)O] | (2S,3R)-2-amino-6-borono-3- (ureidomethyl)hexanoic acid |
| 8 | 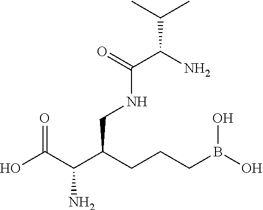 [SMILES: CC(C)[C@H](N)C(=O)NC[C@@H](CCCB(O)O)[C@H](N)C(=O)O] | (2S,3R)-2-amino-3-(((S)-2-amino-3- methylbutanamido)methyl)-6- boronohexanoic acid |
| 9 | 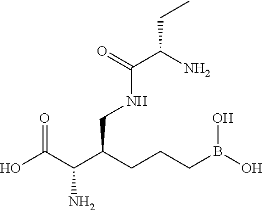 [SMILES: CC[C@H](N)C(=O)NC[C@@H](CCCB(O)O)[C@H](N)C(=O)O] | (2S,3R)-2-amino-3-(((S)-2- aminobutanamido)methyl)-6- boronohexanoic acid |
| 10 | 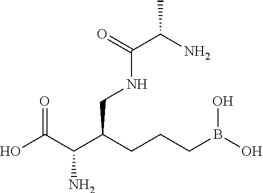 [SMILES: C[C@H](N)C(=O)NC[C@@H](CCCB(O)O)[C@H](N)C(=O)O] | (2S,3R)-2-amino-3-(((S)-2- aminopropanamido)methyl)-6- boronohexanoic acid |
| 11 | 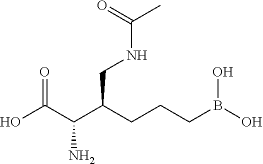 [SMILES: CC(=O)NC[C@@H](CCCB(O)O)[C@H](N)C(=O)O] | (2S,3R)-3-(acetamidomethyl)-2-amino-6- boronohexanoic acid |
| 12 | 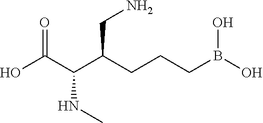 | (2S,3R)-3-(aminomethyl)-6-borono-2- (methylamino)hexanoic acid |
| 13 | 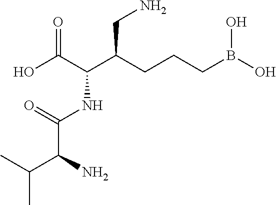 [SMILES: CC(C)[C@H](N)C(=O)N[C@H](C(=O)O)[C@@H](CN)CCCB(O)O] | (2S,3R)-2-((S)-2-Amino-3- methylbutanamido)-3-(aminomethyl)-6- boronohexanoic acid |
| 14 | 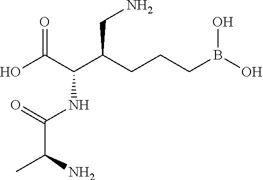 [SMILES: C[C@H](N)C(=O)N[C@H](C(=O)O)[C@@H](CN)CCCB(O)O] | (2S,3R)-3-(aminomethyl)-2-((S)-2- aminopropanamido)-6-boronohexanoic acid |
| 15 | 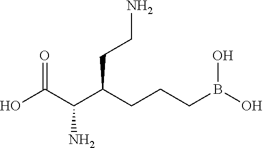 [SMILES: NCC[C@@H](CCCB(O)O)[C@H](N)C(=O)O] | (2S,3R)-2-amino-3-(2-aminoethyl)-6- boronohexanoic acid dihydrochoride |
| 16 | 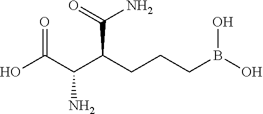 [SMILES: NC(=O)[C@@H](CCCB(O)O)[C@H](N)C(=O)O] | (2S,3S)-2-amino-6-borono-3- carbamoylhexanoic acid |
| 17 | 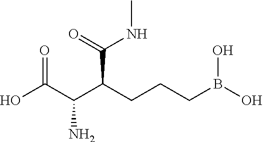 [SMILES: CNC(=O)[C@@H](CCCB(O)O)[C@H](N)C(=O)O] | (2S,3S)-2-amino-6-borono-3- (methylcarbamoyl)hexanoic acid |
| 18 | 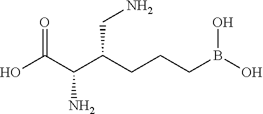 [SMILES: NC[C@H](CCCB(O)O)[C@H](N)C(=O)O] | (2S,3S)-2-amino-3-(aminomethyl)-6- boronohexanoic acid |
| 19 | 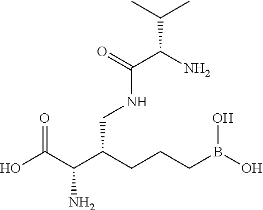 [SMILES: CC(C)[C@H](N)C(=O)NC[C@H](CCCB(O)O)[C@H](N)C(=O)O] | (2S,3S)-2-amino-3-(((S)-2-amino-3- methylbutanamido)methyl)-6- boronohexanoic acid |
| 20 | 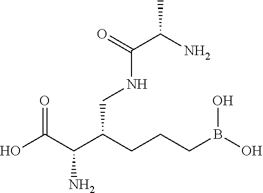 [SMILES: C[C@H](N)C(=O)NC[C@H](CCCB(O)O)[C@H](N)C(=O)O] | (2S,3S)-2-amino-3-(((S)-2- aminopropanamido)methyl)-6- boronohexanoic acid |
| 21 | 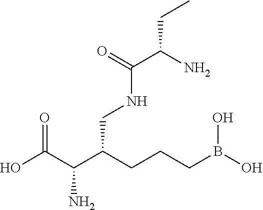 [SMILES: CC[C@H](N)C(=O)NC[C@H](CCCB(O)O)[C@H](N)C(=O)O] | (2S,3S)-2-amino-3-(((S)-2- aminobutanamido)methyl)-6- boronohexanoic acid |
| 22 | 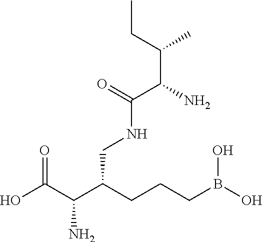 [SMILES: CC[C@H](C)[C@H](N)C(=O)NC[C@H](CCCB(O)O)[C@H](N)C(=O)O] | (2S,3S)-2-amino-3-(((2S,3S)-2-amino-3- methylpentanamido)methyl)-6- boronohexanoic acid |
| 23 | 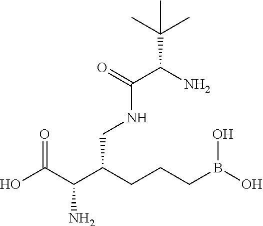 [SMILES: CC(C)(C)[C@H](N)C(=O)NC[C@H](CCCB(O)O)[C@H](N)C(=O)O] | (2S,3S)-2-amino-3-(((S)-2-amino-3,3- dimethylbutanamido)methyl)-6- boronohexanoic acid |
| 24 | 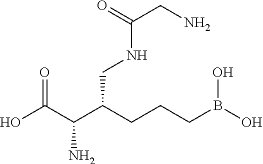 [SMILES: NCC(=O)NC[C@H](CCCB(O)O)[C@H](N)C(=O)O] | (2S,3S)-2-amino-3-((2- aminoacetamido)methyl)-6-boronohexanoic acid |
| 25 | 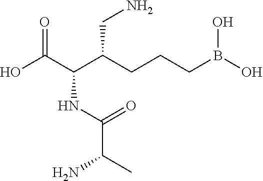 [SMILES: C[C@H](N)C(=O)N[C@H](C(=O)O)[C@H](CN)CCCB(O)O] | (2S,3S)-3-(aminomethyl)-2-[[(2S)-2- aminopropanoyl]amino]-6-borono-hexanoic acid |
| 26 | 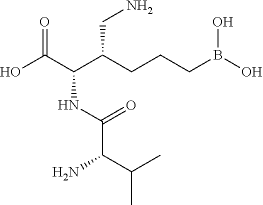 [SMILES: CC(C)[C@H](N)C(=O)N[C@H](C(=O)O)[C@H](CN)CCCB(O)O] | (2S,3S)-3-(aminomethyl)-2-[[(2S)-2-amino- 3-methyl-butanoyl]amino]-6-borono- hexanoic acid |

The language “C₁-C₄ alkyl” includes acyclic saturated alkyl moieties having 1-4 carbon atoms. Examples of C₁-C₄ alkyl moieties include methyl, ethyl, n-propyl, isopropyl, n-butyl, sec-butyl, isobutyl, and tert-butyl.

The language “nitrogen-containing six-membered heterocycle” includes saturated cycloalkyl moieties having at least one carbon replaced with nitrogen. Examples of nitrogen-containing six-membered heterocycles include piperidine, piperazine, morpholine, thiomorpholine and hexahydro-1,3,5-triazine.

The language “pharmaceutically acceptable salt” includes acid addition or base addition salts that retain the biological effectiveness and properties of the compounds of formula (I), (Ia), (Ib), (II), (III), (IV) and Table 1 and, which typically are not biologically or otherwise undesirable.

In many cases, the compounds of formula (I), (Ia), (Ib), (II), (III), (IV) and Table 1 are capable of forming acid and/or base salts by virtue of the presence of basic and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethanedisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulfate, naphthoate, napsylate, nicotinate, nitrate, octadecanoate, oleate, oxalate, palmitate, palmoate, phosphate/hydrogen phosphate/dihydrogen phosphate, polygalacturonate, propionate, stearate, succinate, subsalicylate, sulfate/hydrogensulfate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoroacetic acid, sulfosalicylic acid, and the like.

Pharmaceutically acceptable base addition salts can be formed with inorganic and organic bases. Inorganic bases from which salts can be derived include, for example, ammonia and salts of ammonium and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the compounds of formula (I), (Ia), (Ib), (II), (III), (IV) and Table 1 can be synthesized from a basic or acidic moiety, by conventional chemical methods. Generally, such salts can be prepared by reacting free acid forms of these compounds with a stoichiometric amount of the appropriate base (such as Na⁺, Ca²⁺, Mg²⁺, or K⁺ hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences,” 20th ed., Mack Publishing Company, Easton, Pa., (1985); Berge et al., “J. Pharm. Sci., 1977, 66, 1-19 and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms for the compounds of formula (I), (Ia), (Ib), (II), (III), (IV) and Table 1, or pharmaceutically acceptable salts thereof. Isotopically labeled compounds have structures depicted by the formulas given herein except that one or more atoms are replaced by an atom of the same element but with differing mass number. Examples of isotopes that can be incorporated into the compounds of formula (I), (Ia), (Ib), (II), (III), (IV) and Table 1 and their pharmaceutically acceptable salts include isotopes of hydrogen, carbon, nitrogen, oxygen, phosphorous, sulfur, fluorine, chlorine and iodine, such as ²H, ³H, ¹¹C, ¹³C, ¹⁴C, ¹⁵N, ³⁵S, ³⁶Cl and ¹²⁵I. Isotopically labeled compounds of formula (I), (Ia), (Ib), (II), (III), (IV) and Table 1 can generally be prepared by conventional techniques known to those skilled in the art or by processes analogous to those described in the accompanying Examples using appropriate isotopically labeled reagents in place of the non-labeled reagents previously employed.

The compounds of formula (I), (Ia), (Ib), (II), (III), (IV) and Table 1, or pharmaceutically acceptable salts thereof, may have different isomeric forms. The language “optical isomer,” “stereoisomer” or “diastereoisomer” refers to any of the various stereoisomeric configurations which may exist for a given compound of formula (I), (Ia), (Ib), (II), (III), (IV) and Table 1, or a pharmaceutically acceptable salt thereof. It is understood that a substituent may be attached at a chiral center of a carbon atom and, therefore, the disclosed compounds include enantiomers, diastereomers and racemates. The term “enantiomer” includes pairs of stereoisomers that are non-superimposable mirror images of each other. A 1:1 mixture of a pair of enantiomers is a racemic mixture. The term is used to designate a racemic mixture where appropriate. The terms “diastereomers” or “diastereoisomers” include stereoisomers that have at least two asymmetric atoms, but which are not mirror images of each other. The absolute stereochemistry is specified according to the Cahn-Ingold-Prelog R—S system. When a compound is a pure enantiomer, the stereochemistry at each chiral center may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds of formula (I), (Ia), (Ib), (II), (III), (IV) and Table 1, or a pharmaceutically acceptable salt thereof, contain one or more asymmetric centers or axes and may thus give rise to enantiomers, diastereomers or other stereoisomeric forms that may be defined, in terms of absolute stereochemistry, as (R)- or (S)-. The present disclosure is meant to include all such possible isomers, including racemic mixtures, optically pure forms and intermediate mixtures. Optically active (R)- and (S)-isomers may be prepared using chiral synthons or chiral reagents, or resolved using conventional techniques well known in the art, such as chiral HPLC.

Also disclosed herein the Intermediates 1 to 56 in the Examples, and salts thereof.

### Pharmaceutical Compositions

In some embodiments, disclosed are pharmaceutical compositions comprising a compound of formula (I), (Ia), (Ib), (II), (III), (IV) or Table 1, or a pharmaceutically acceptable salt thereof, and a pharmaceutically acceptable carrier.

The language “pharmaceutically acceptable carrier” includes compounds, materials, compositions, and/or dosage forms which are, within the scope of sound medical judgment, suitable for use in contact with the tissues of human beings and animals without excessive toxicity, irritation, allergic response, or other problem or complication, as ascertained by one of skill in the art.

The disclosed compositions may be in a form suitable for oral use (for example, as tablets, lozenges, hard or soft capsules, aqueous or oily suspensions, emulsions, dispersible powders or granules, syrups or elixirs), for topical use (for example, as creams, ointments, gels, or aqueous or oily solutions or suspensions), for administration by inhalation (for example, as a finely divided powder or a liquid aerosol), for administration by insufflation (for example, as a finely divided powder) or for parenteral administration (for example, as a sterile aqueous or oily solution for intravenous, subcutaneous, intramuscular or intramuscular dosing or as a suppository for rectal dosing).

The amount of active ingredient that is combined with one or more pharmaceutically acceptable carriers to produce a single dosage form will necessarily vary depending upon the host treated and the particular route of administration. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

### Therapeutic Utilities

The present compounds are useful as arginase inhibitors in therapies.

In one aspect, disclosed are methods for treating cancer in a subject in need thereof, comprising administering to the subject an effective amount of a compound of formula (I), (Ia), (Ib), (II), (III), (IV) or Table 1, or a pharmaceutically acceptable salt thereof.

In one aspect, disclosed are methods for treating a respiratory inflammatory disease in a subject in need thereof, comprising administering to the subject an effective amount of a compound of formula (I), (Ia), (Ib), (II), (III), (IV) or Table 1, or a pharmaceutically acceptable salt thereof.

In one aspect, disclosed is a compound of formula (I), (Ia), (Ib), (II), (III), (IV) or Table 1, or a pharmaceutically acceptable salt thereof, for use in treating cancer.

In one aspect, disclosed is a compound of formula (I), (Ia), (Ib), (II), (III), (IV) or Table 1, or a pharmaceutically acceptable salt thereof, for use in treating a respiratory inflammatory disease.

In one aspect, disclosed is the use of a compound of formula (I), (Ia), (Ib), (II), (III), (IV) or Table 1, or a pharmaceutically acceptable salt, in the manufacture of a medicament for treating cancer.

In one aspect, disclosed is the use of a compound of formula (I), (Ia), (Ib), (II), (III), (IV) or Table 1, or a pharmaceutically acceptable salt, in the manufacture of a medicament for treating a respiratory inflammatory disease.

In one aspect, disclosed are pharmaceutical compositions comprising a compound of formula (I), (Ia), (Ib), (II), (III), (IV) or Table 1, or a pharmaceutically acceptable salt thereof, for use in treating cancer.

In one aspect, disclosed are pharmaceutical compositions comprising a compound of formula (I), (Ia), (Ib), (II), (III), (IV) or Table 1, or a pharmaceutically acceptable salt thereof, for use in treating a respiratory inflammatory disease.

The term “cancer” includes, for example, renal cell carcinoma, head and neck squamous cell carcinoma, lung cancer (e.g., small cell lung cancer (SCLC), non-small cell lung cancer (NSCLC), mesothelioma), pancreatic cancer, colorectal cancer, breast cancer, acute myeloid leukemia (AML), prostate cancer, gastric cancer, bladder cancer, melanoma, renal cancer and ovarian cancer. In some embodiments, the cancer has metastasized. In some embodiments, the cancer is associated with Arginase 1 and/or Arginase 2 modulation.

In some embodiments, the cancer is associated with increased plasma Arginase 1 levels. In some embodiments, the cancer is associated with decreased plasma arginine levels. In some embodiments, the cancer is associated with both increased plasma Arginase 1 levels and decreased plasma arginine levels. In some embodiments, the cancer associated with increased plasma Arginase 1 levels and/or decreased plasma arginine levels includes renal cell carcinoma, head and neck squamous cell carcinoma, lung cancer (e.g., small cell lung cancer (SCLC), non-small cell lung cancer (NSCLC), mesothelioma), pancreatic cancer, colorectal cancer and breast cancer.

In some embodiments, the cancer secretes Arginase 2, for example, acute myeloid leukemia and prostate cancer.

In some embodiments, the cancer is associated with Arginase 1 positive tumor infiltrating immune cells, for example, lung cancer (small cell lung cancer (SCLC), non-small cell lung cancer (NSCLC), gastric cancer, bladder cancer, colorectal cancer, melanoma, head and neck squamous cell carcinoma, breast cancer, prostate cancer, ovarian cancer, pancreatic cancer and renal cancer.

The term “a respiratory inflammatory disease” refers to inflammatory conditions or disorders that affect the airspaces, pulmonary vasculature, pulmonary interstitium, or a combination thereof. They can be isolated to the lung or involve multiple organs. In one embodiment, the respiratory inflammatory disease is an inflammatory lung disease. In another embodiment, the inflammatory lung disease is noninfectious. In some embodiments, the respiratory inflammatory disease is associated with Arginase 1 and/or Arginase 2 modulation.

In some embodiments, the respiratory inflammatory disease is asthma, chronic obstructive pulmonary disease (COPD), chemically-induced lung fibrosis, idiopathic pulmonary fibrosis, cystic fibrosis, or a combination thereof. In some embodiments, the respiratory inflammatory disease is chronic obstructive pulmonary disease (COPD) or asthma.

In one aspect, disclosed are methods for inhibiting arginase in a subject in need thereof, comprising administering to the subject an effective amount of a compound of formula (I), (Ia), (Ib), (II), (III), (IV) or Table 1, or a pharmaceutically acceptable salt thereof.

In one aspect, disclosed is are compounds of formula (I), (Ia), (Ib), (II), (III), (IV) and Table 1, or a pharmaceutically acceptable salt thereof, for use in inhibiting arginase.

In one aspect, disclosed is the use of a compound of formula (I), (Ia), (Ib), (II), (III), (IV) or Table 1, or a pharmaceutically acceptable salt thereof, in the manufacture of a medicament for inhibiting arginase.

In one aspect, disclosed are pharmaceutical compositions comprising a compound of formula (I), (Ia), (Ib), (II), (III), (IV) or Table 1, or a pharmaceutically acceptable salt thereof, for use in inhibiting arginase.

The term “arginase” includes manganese-containing enzymes belonging to the ureahydrolase family that catalyze the fifth and final step in the urea cycle converting L-arginine into L-ornithine and urea. The term “arginase” includes the two isozymes of the enzyme, e.g., Arginase 1, which functions in the urea cycle, and is located primarily in the cytoplasm of the liver, and Arginase 2, which is located in the mitochondria of several tissues in the body and is implicated in the regulation of arginine/ornithine concentrations in the cell. In some embodiments, the compounds of formula (I), (Ia), (Ib), (II), (III), (IV) and Table 1, or a pharmaceutically acceptable salt thereof, are selective for arginase 1. In some embodiments, the compounds of formula (I), (Ia), (Ib), (II), (III), (IV) and Table 1, or a pharmaceutically acceptable salt thereof, are selective for Arginase 2. In some embodiments, the compounds of formula (I), (Ia), (Ib), (II), (III), (IV) and Table 1, or a pharmaceutically acceptable salt thereof, inhibit both Arginase 1 and Arginase 2.

The language “effective amount” includes an amount of a compound of formula (I), (Ia), (Ib), (II), (III), (IV) or Table 1, or a pharmaceutically acceptable salt thereof, that will elicit a biological or medical response in a subject, for example, the reduction or inhibition of enzyme or protein activity related to arginase or cancer, amelioration of symptoms of cancer or the slowing or delaying of progression of cancer. In some embodiments, the language “effective amount” includes the amount of a compound of formula (I), (Ia), (Ib), (II), (III), (IV) or Table 1, or a pharmaceutically acceptable salt thereof, that when administered to a subject, is effective to at least partially alleviate, inhibit, and/or ameliorate cancer or inhibit arginase, and/or reduce or inhibit the growth of a tumor or proliferation of cancerous cells in a subject.

The term “subject” includes warm blooded mammals, for example, primates, dogs, cats, rabbits, rats, and mice. In some embodiments, the subject is a primate, for example, a human. In some embodiments, the subject is suffering from cancer. In some embodiments, the subject is in need of treatment (e.g., the subject would benefit biologically or medically from treatment). In some embodiments, the subject has increased plasma Arginase 1 levels. In some embodiments, the subject has decreased arginine levels. In some embodiments, the patient has both increased plasma Arginase 1 levels and decreased arginine levels. In some embodiments, the subject has a cancer secreting Arginase 2 (e.g., acute myeloid leukemia or prostate cancer). In some embodiments, the subject has Arginase 1 positive tumor infiltrating immune cells.

The language “inhibit,” “inhibition” or “inhibiting” includes a decrease in the baseline activity of a biological activity or process. In some embodiments, the compounds of formula (I), (Ia), (Ib), (II), (III), (IV) and Table 1, or a pharmaceutically acceptable salt thereof inhibit arginase.

The language “treat,” “treating” and “treatment” includes the reduction or inhibition of enzyme or protein activity related to arginase or in a subject, amelioration of one or more symptoms of a cancer, or the slowing or delaying of progression of cancer in a subject. The language “treat,” “treating” and “treatment” also includes the reduction or inhibition of the growth of a tumor or proliferation of cancerous cells in a subject.

### EXAMPLES

Aspects of the present disclosure can be further defined by reference to the following non-limiting examples, which describe in detail preparation of certain compounds and intermediates of the present disclosure and methods for using compounds of the present disclosure. It will be apparent to those skilled in the art that many modifications, both to materials and methods, can be practiced without departing from the scope of the present disclosure.

Unless stated otherwise:

(i) all syntheses were carried out at ambient temperature, i.e. in the range 17 to 25° C. and under an atmosphere of an inert gas such as nitrogen unless otherwise stated;

(ii) evaporations were carried out by rotary evaporation or utilising Genevac equipment or Biotage v10 evaporator in vacuo and work-up procedures were carried out after removal of residual solids by filtration;

(iii) flash chromatography purifications were performed on an automated Teledyne Isco CombiFlash® Rf or Teledyne Isco CombiFlash® Companion® using prepacked RediSep Rf Gold™ Silica Columns (20-40 μm, spherical particles), GraceResolv™ Cartridges (Davisil® silica) or Silicycle cartridges (40-63 μm).

(iv) preparative chromatography was performed on a Gilson prep HPLC instrument with UV collection; alternatively, preparative chromatography was performed on a Waters AutoPurification HPLC-MS instrument with MS- and UV-triggered collection;

(v) chiral preparative chromatography was performed on a Gilson instrument with UV collection (233 injector/fraction collector, 333 & 334 pumps, 155 UV detector) or a Varian Prep Star instrument (2×SD1 pumps, 325 UV detector, 701 fraction collector) pump running with Gilson 305 injection; alternatively, chiral preparative chromatography was performed on a Waters Prep 100 SFC-MS instrument with MS- and UV-triggered collection or a Thar MultiGram III SFC instrument with UV collection.

(vi) yields, where present, are not necessarily the maximum attainable;

(vii) in general, the structures of end-products of the Formula I were confirmed by nuclear magnetic resonance (NMR) spectroscopy; NMR chemical shift values were measured on the delta scale [proton magnetic resonance spectra were determined using a Bruker Avance 500 (500 MHz), Bruker Avance 400 (400 MHz), Bruker Avance 300 (300 MHz) or Bruker DRX (300 MHz) instrument]; measurements were taken at ambient temperature unless otherwise specified; the following abbreviations have been used: s, singlet; d, doublet; t, triplet; q, quartet; m, multiplet; dd, doublet of doublets; ddd, doublet of doublet of doublet; dt, doublet of triplets; bs, broad signal.

(viii) in general, end-products of the Formula I were also characterized by mass spectroscopy following liquid chromatography (LCMS or UPLC); UPLC was carried out using a Waters UPLC fitted with a Waters SQ mass spectrometer (Column temp 40° C., UV=220-300 nm or 190-400 nm, Mass Spec=ESI with positive/negative switching) at a flow rate of 1 mL/min using a solvent system of 97% A+3% B to 3% A+97% B over 1.50 min (total run time with equilibration back to starting conditions, etc., 1.70 min), where A=0.1% formic acid or 0.05% trifluoroacetic acid in water (for acidic work) or 0.1% ammonium hydroxide in water (for basic work) and B=acetonitrile. For acidic analysis the column used was a Waters Acquity HSS T3 (1.8 μm, 2.1×50 mm), for basic analysis the column used was a Waters Acquity BEH C18 (1.7 μm 2.1×50 mm). Alternatively, UPLC was carried out using a Waters UPLC fitted with a Waters SQ mass spectrometer (Column temp 30° C., UV=210-400 nm, Mass Spec=ESI with positive/negative switching) at a flow rate of 1 mL/min using a solvent gradient of 2 to 98% B over 1.5 mins (total run time with equilibration back to starting conditions 2 min), where A=0.1% formic acid in water and B=0.1% formic acid in acetonitrile (for acidic work) or A=0.1% ammonium hydroxide in water and B=acetonitrile (for basic work). For acidic analysis the column used was a Waters Acquity HSS T3 (1.8 μm, 2.1×30 mm), for basic analysis the column used was a Waters Acquity BEH C18 (1.7 μm, 2.1×30 mm); LCMS was carried out using a Waters Alliance HT (2795) fitted with a Waters ZQ ESCi mass spectrometer and a Phenomenex Gemini-NX C18 (5 μm, 110 A, 2.1×50 mm column at a flow rate of 1.1 mL/min 95% A to 95% B over 4 min with a 0.5 min hold where A=0.1% formic acid and B=0.1% formic acid in acetonitrile (for acidic work) or A=0.1% ammonium hydroxide in water and B=acetonitrile (for basic work). Additionally, LCMS was carried out using a Shimadzu UFLC fitted with a Shimadzu LCMS-2020 mass spectrometer and a Waters HSS C18 (1.8 μm, 2.1×50 mm) or Shim-pack XR-ODS (2.2 μm, 3.0×50 mm) or Phenomenex Gemini-NX C18 (3 μm, 3.0×50 mm) column at a flow rate of 0.7 mL/min (for Waters HSS C18 column), 1.0 mL/min (for Shim-pack XR-ODS column) or 1.2 mL/min (for Phenomenex Gemini-NX C18), 95% A to 95% B over 2.2 min with a 0.6 min hold, where A=0.1% formic acid or 0.05% trifluoroacetic acid in water (for acidic work) or 0.1% ammonium hydroxide or 6.5 mM ammonium carbonate in water (for basic work) and B=acetonitrile. The reported molecular ion corresponds to the [M+H]+ unless otherwise specified; for molecules with multiple isotopic patterns (Br, Cl, etc.) the reported value is the one obtained for the lowest isotope mass unless otherwise specified.

(ix) ion exchange purification was generally performed using an SCX-2 (Biotage) cartridge.

(x) intermediate purity was assessed by thin layer chromatographic, mass spectroscopy, LCMS, UPLC/MS, HPLC (high performance liquid chromatography) and/or NMR analysis;

(xi) the following abbreviations have been used:
EtOAc: ethyl acetate
Et₂O: diethyl ether
DMSO: dimethylsulfoxide
LAH: lithium aluminum hydride
LiHMDS: lithium hexamethyldisilazane
MeOH: methanol
TFA: trifluoroacetic acid
MeCN: acetonitrile
LCMS: liquid chromatography-mass spectrometry
rt or RT: room temperature
aq: aqueous
THF: tetrahydrofuran
DCM: dichloromethane
DMF: dimethylformamide
HATU: (1-[Bis(dimethylamino)methylene]-1H-1,2,3-triazolo[4,5-b]pyridinium 3-oxid hexafluorophosphate)
TBAF: tetrabutylammonium fluoride
AcOH: acetic acid
DIAD: diisopropyl azodicarboxylate
Boc-Ala-OH: N-(tert-butoxycarbonyl)-L-alanine
Boc-Val-OH: N-(tert-butoxycarbonyl)-L-valine
HEPES: (4-(2-hydroxyethyl)-1-piperazineethanesulfonic acid)

### Example 1: (2S,3R)-2-amino-3-(aminomethyl)-6-Boronohexanoic Acid Dihydrochloride

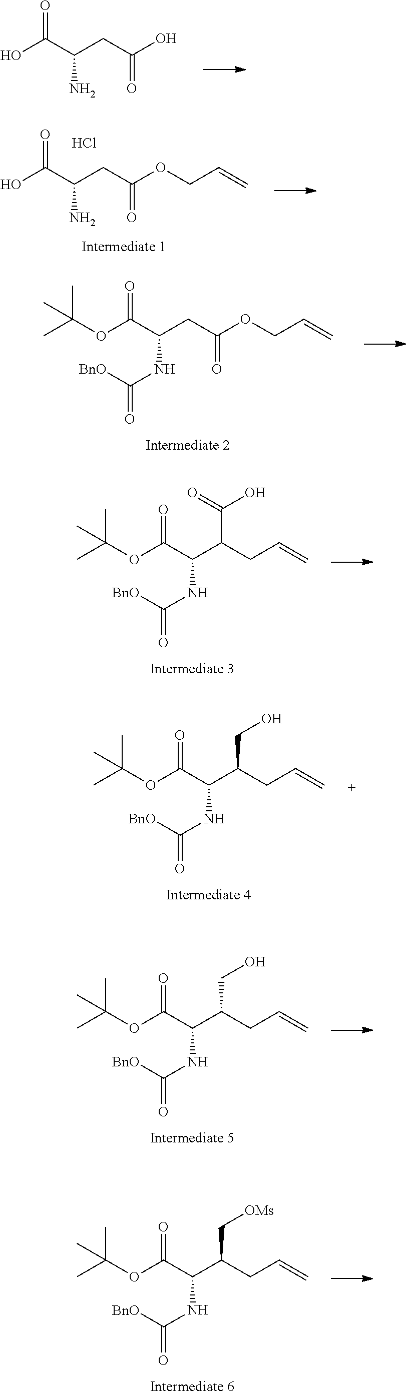

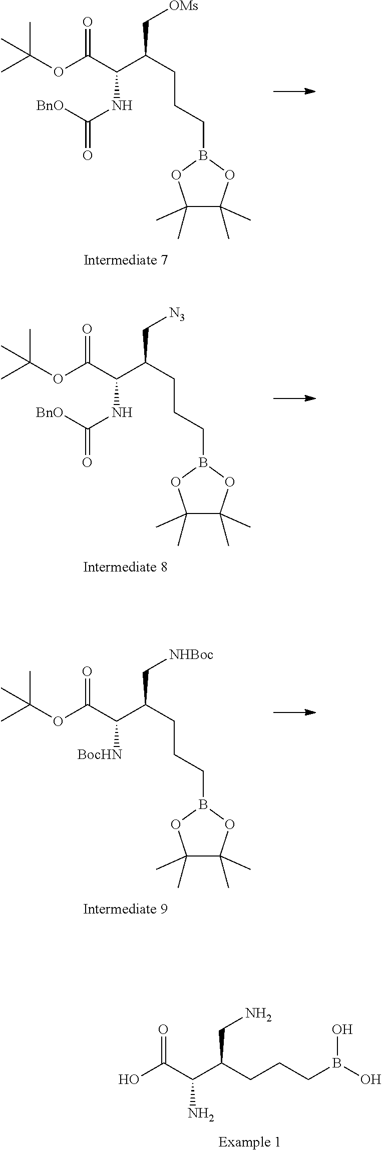

### Intermediate 1: (S)-4-(allyloxy)-2-amino-4-oxobutanoic Acid Hydrochloride

L-Aspartic acid (10.66 g, 80.09 mmol) was suspended in allyl alcohol (60.0 mL, 880 mmol) under an atmosphere of N₂. Chlorotrimethylsilane (31.0 mL, 240 mmol) was added dropwise to the suspension via syringe pump at a rate of 1 mL/min. The reaction mixture stirred at room temperature for 16 h. The reaction was diluted with ice-cold Et₂O (100 mL) and the suspension was filtered. The solid was washed with ice-cold Et₂O (3×15 mL) and dried to afford (S)-4-(allyloxy)-2-amino-4-oxobutanoic acid hydrochloride (Intermediate 1, 12.7 g, 76% yield) as an amorphous white solid, which was carried forward without further purification. ¹H NMR (300 MHz, D₂O) δ 3.14 (2H, d), 4.25 (1H, t), 4.70 (2H, d), 5.26-5.48 (2H, m), 5.89-6.08 (1H, m); m/z: (ES⁺) [M+H]⁺=174.

### Intermediate 2: (S)-4-allyl 1-tert-butyl 2-(benzyloxycarbonylamino)succinate

(S)-4-(allyloxy)-2-amino-4-oxobutanoic acid hydrochloride (Intermediate 1, 11.58 g, 55.24 mmol) was dissolved in water (100 mL) and 1,4-dioxane (100 mL). Sodium carbonate (23.0 g, 220 mmol) was added portionwise at room temperature and the reaction was stirred for 5 min. Benzyl chloroformate (8.3 mL, 58 mmol) was added dropwise to the reaction via syringe pump at a rate of 1 mL/min. The biphasic reaction mixture stirred at room temperature for 4 h. The crude reaction was quenched with concentrated aqueous HCl until the pH was <1. The layers were separated and the aqueous layer was extracted with EtOAc (2×25 mL). The combined organics were dried over MgSO₄, filtered and concentrated to afford a colorless oil. The crude carboxylic acid was dissolved in DCM (100 mL) and cooled to −78° C. in a pressure flask. Sulfuric acid (3.0 mL, 56 mmol) was added, followed immediately by pre-condensed isobutylene (66.0 mL, 710 mmol). The flask was sealed and stirred for 3 d, while the ice bath was allowed to expire. The reaction was poured onto saturated aqueous sodium bicarbonate (200 mL) and stirred 30 min. The layers were separated, and the aqueous layer was extracted with DCM (2×20 mL). The combined organics were dried over MgSO₄, filtered and concentrated to dryness. The crude material was purified by silica gel chromatography (hexanes/EtOAc) to afford (S)-4-allyl 1-tert-butyl 2-(benzyloxycarbonylamino)succinate (Intermediate 2, 12.2 g, 61% yield) as a colorless oil. ¹H NMR (300 MHz, CDCl₃) δ 1.47 (9H, s), 2.76-2.93 (1H, dd), 2.94-3.12 (1H, dd), 4.48-4.57 (1H, m), 4.60 (2H, dq), 5.14 (2H, s), 5.26 (1H, dq), 5.33 (1H, dq), 5.71 (1H, br d), 5.91 (1H, ddt), 7.31-7.48 (5H, m); m/z: (ES⁺) [M+H]⁺=381.

### Intermediate 3: 2-((S)-1-(benzyloxycarbonylamino)-2-tert-butoxy-2-oxoethyl)ent-4-enoic Acid

A solution of LiHMDS (1 M in toluene, 100 mL, 100 mmol) was added to an oven-dried multineck flask and diluted with THF (50 mL) under an atmosphere of N₂. The solution was cooled to −78° C. and (S)-4-allyl 1-tert-butyl 2-(benzyloxycarbonylamino)succinate (Intermediate 2, 12.2 g, 33.5 mmol) was added dropwise to the reaction flask as a solution in THF (50 mL). The reaction stirred at −78° C. for 80 min. Chlorotrimethylsilane (17.0 mL, 133 mmol) was added and the reaction stirred at −78° C. for an additional 1 h. The reaction was then heated to 60° C. for 160 min. The reaction mixture was cooled to room temperature and quenched with 2 M aq. HCl (67 mL). After stirring vigorously for 30 min, the layers were separated and the aqueous layer was extracted with EtOAc (2×30 mL). The combined organics were dried over MgSO₄, filtered and concentrated to dryness. The crude material was purified by silica gel chromatography (DCM/MeOH) to afford 2-((S)-1-(benzyloxycarbonylamino)-2-tert-butoxy-2-oxoethyl)pent-4-enoic acid (Intermediate 3, 12.0 g, 99%) as an inseparable mixture of diasteromers in a ˜1.7:1 ratio. ¹H NMR (300 MHz, CDCl₃) δ 1.41 (3.4H, s) 1.43 (5.6H, s), 2.19-2.43 (1H, m), 2.44-2.66 (1H, m), 2.81-2.99 (0.65H, m), 3.08-3.25 (0.35H, m), 4.48-4.63 (1H, m), 4.97-5.20 (4H, m), 5.52-5.71 (1H, m), 5.71-5.94 (1H, m), 7.25-7.39 (5H, m); m/z: (ES⁺) [M+H]⁺=364.

### Intermediate 4: (2S,3S)-tert-butyl 2-(benzyloxycarbonylamino)-3-(hydroxymethyl)hex-5-enoate and

### Intermediate 5: (2S,3R)-tert-butyl 2-(benzyloxycarbonylamino)-3-(hydroxymethyl)hex-5-enoate

2-((S)-1-(benzyloxycarbonylamino)-2-tert-butoxy-2-oxoethyl)pent-4-enoic acid (Intermediate 3, 12.0 g, 33.0 mmol) was dissolved in THF (60 mL) and cooled to −10° C. under an atmosphere of N₂. N-Methylmorpholine (3.7 mL, 34 mmol) was added and the reaction stirred at −10° C. for 5 min followed by addition of ethyl chloroformate (3.2 mL, 33 mmol). The reaction mixture was warmed to room temperature and stirred for 40 min. The resulting suspension was filtered directly into a solution of sodium borohydride (3.0 g, 79 mmol) in water (60 mL) at 0° C. Following addition, the reaction was warmed to room temperature and stirred for 3 h. The reaction mixture was cooled to 0° C. and carefully quenched with 2 M aq. HCl (20 mL). The layers were separated and the aqueous layer was extracted with EtOAc (2×25 mL). The combined organics were dried over MgSO₄, filtered and concentrated to dryness. The crude material was purified by silica gel chromatography (hexanes/EtOAc) to afford (2S,3S)-tert-butyl 2-(benzyloxycarbonylamino)-3-(hydroxymethyl)hex-5-enoate (Intermediate 4, 5.77 g, 50% yield) and (2S,3R)-tert-butyl 2-(benzyloxycarbonylamino)-3-(hydroxymethyl)hex-5-enoate (Intermediate 5, 3.96 g, 34% yield) as colorless oils.

Intermediate 4: ¹H NMR (300 MHz, CDCl₃) δ 1.46 (9H, s), 1.70-1.85 (1H, m), 1.85-1.99 (1H, m), 2.18-2.37 (1H, m), 3.26 (1H, t), 3.63 (1H, dd), 4.58 (1H, dd), 4.92-5.06 (2H, m), 5.10 (2H, s), 5.55 (1H, br d), 5.60-5.78 (1H, m), 7.26-7.39 (5H, m); m/z: (ES⁺) [M+H]⁺=350.
Intermediate 5: ¹H NMR (300 MHz, CDCl₃) δ 1.45 (9H, s), 2.03-2.12 (1H, m), 2.13-2.23 (2H, m), 3.65 (2H, qd), 4.34 (1H, br dd), 5.00-5.15 (4H, m), 5.62-5.90 (2H, m), 7.25-7.41 (5H, m); m/z: (ES⁺) [M+H]⁺=350.

### Intermediate 6: (2S,3S)-tert-butyl 2-(benzyloxycarbonylamino)-3-((methylsulfonyloxy)methyl)hex-5-enoate

Triethylamine (7.4 mL, 53 mmol) and methanesulfonyl chloride (2.6 mL, 33 mmol) were added sequentially to a solution of (2S,3S)-tert-butyl 2-(benzyloxycarbonylamino)-3-(hydroxymethyl)hex-5-enoate (Intermediate 4, 4.61 g, 13.2 mmol) in DCM (100 mL) at 0° C. The reaction was warmed to room temperature and stirred for 90 min. The crude mixture was diluted with DCM (25 mL) and washed sequentially with saturated aqueous sodium bicarbonate, water, and brine (25 mL each). The organic layer was dried over MgSO₄, filtered and concentrated to dryness. The crude material was purified by silica gel chromatography (hexanes/EtOAc) to afford (2S,3S)-tert-butyl 2-(benzyloxycarbonylamino)-3-((methylsulfonyloxy)methyl)hex-5-enoate (Intermediate 6, 5.3 g, 93% yield) as a pale yellow oil. ¹H NMR (300 MHz, CDCl₃) δ 1.46 (9H, s), 1.96-2.19 (2H, m), 2.36-2.58 (1H, m), 2.97 (3H, s), 4.00-4.23 (2H, m), 4.52 (1H, br d), 5.00-5.17 (4H, m), 5.34 (1H, br d), 5.60-5.83 (1H, m), 7.27-7.37 (5H, m); m/z: (ES⁺) [M+H]⁺=445.

### Intermediate 7: (2S,3S)-tert-butyl 2-(benzyloxycarbonylamino)-3-((methylsulfonyloxy)methyl)-6-(4,4,5,5-tetramethyl-1,3,2-dioxaborolan-2-yl)hexanoate

Bis(1,5-cyclooctadiene)diiridium (I) dichloride (0.25 g, 0.37 mmol) and bis(diphenylphosphino)methane (0.28 g, 0.74 mmol) were added to an oven-dried round-bottom flask. The flask was sealed and purged with N₂. The solids were dissolved in DCM (35 mL) and 4,4,5,5-tetramethyl-1,3,2-dioxaborolane (4.00 mL, 28.0 mmol) was added slowly to the solution. The reaction stirred at room temperature for 10 min. (2S,3S)-tert-butyl 2-(benzyloxycarbonylamino)-3-((methylsulfonyloxy)methyl)hex-5-enoate (Intermediate 6, 5.27 g, 12.3 mmol) was added to the reaction as a solution in DCM (30 mL) and the reaction mixture stirred overnight. The reaction mixture was cooled to 0° C. and quenched with MeOH (6 mL) and water (50 mL). The layers were separated and the aqueous layer was extracted with DCM (2×15 mL). The combined organics were dried over MgSO₄, filtered and concentrated to dryness. The crude material was purified by silica gel chromatography (hexanes/EtOAc) to afford (2S,3S)-tert-butyl 2-(benzyloxycarbonylamino)-3-((methylsulfonyloxy)methyl)-6-(4,4,5,5-tetramethyl-1,3,2-dioxaborolan-2-yl)hexanoate (Intermediate 7, 5.84 g, 85% yield) as a yellow gum. ¹H NMR (300 MHz, CDCl₃) δ 0.73 (2H, t), 1.20 (12H, s), 1.24-1.42 (4H, m), 1.45 (9H, s), 2.30-2.50 (1H, m), 2.97 (3H, s), 3.98 (1H, t), 4.18 (1H, dd), 4.50 (1H, br d), 5.02-5.15 (2H, m), 5.35 (1H, br d), 7.27-7.42 (5H, m); m/z: (ES⁺) [M+NH₄]⁺=573.

### Intermediate 8: (2S,3R)-tert-butyl 3-(azidomethyl)-2-(benzyloxycarbonylamino)-6-(4,4,5,5-tetramethyl-1,3,2-dioxaborolan-2-yl)hexanoate

Sodium azide (3.3 g, 51 mmol) was added to a solution (2S,3S)-tert-butyl 2-(benzyloxycarbonylamino)-3-((methylsulfonyloxy)methyl)-6-(4,4,5,5-tetramethyl-1,3,2-dioxaborolan-2-yl)hexanoate (Intermediate 7, 5.84 g, 10.5 mmol) in DMF (30 mL). The reaction was heated to 55° C. and stirred for 16 h under an atmosphere of N₂. A further portion of sodium azide (200 mg, 3 mmol) was added and the reaction stirred at 55° C. for an additional 4 h. The reaction mixture was cooled to room temperature and diluted with water (100 mL). The layers were separated and the aqueous layer was extracted with ether (3×35 mL). The combined organics were washed with brine (50 mL) and then dried over MgSO₄, filtered and concentrated to dryness. The crude material was purified by silica gel chromatography (hexanes/EtOAc) to afford (2S,3R)-tert-butyl 3-(azidomethyl)-2-(benzyloxycarbonylamino)-6-(4,4,5,5-tetramethyl-1,3,2-dioxaborolan-2-yl)hexanoate (Intermediate 8, 3.52 g, 67% yield) as a colorless oil. ¹H NMR (300 MHz, CDCl₃) δ 0.73 (2H, t), 1.20 (12H, s), 1.22-1.40 (4H, m), 1.45 (9H, s), 2.04-2.20 (1H, m), 3.15-3.30 (1H, m), 3.31-3.43 (1H, m), 4.46 (1H, br d), 5.10 (2H, s), 5.37 (1H, br d), 7.26-7.40 (5H, m); m/z: (ES⁺) [M+NH₄]⁺=520.

### Intermediate 9: (2S,3R)-tert-butyl 2-(tert-butoxycarbonylamino)-3-((tert-butoxycarbonylamino)methyl)-6-(4,4,5,5-tetramethyl-1,3,2-dioxaborolan-2-yl)hexanoate

Pd/C (10% wt, 0.46 g, 0.43 mmol) and di-tert-butyl-dicarbonate (2.50 mL, 10.8 mmol) were added to a solution of (2S,3R)-tert-butyl 3-(azidomethyl)-2-(benzyloxycarbonylamino)-6-(4,4,5,5-tetramethyl-1,3,2-dioxaborolan-2-yl)hexanoate (Intermediate 8, 2.16 g, 4.29 mmol) in EtOAc (15 mL). The flask was equipped with a balloon of H₂ and the suspension stirred at room temperature for 2 h. The reaction mixture was filtered through diatomaceous earth and rinsed with EtOAc. The filtrate was concentrated to a cloudy, colorless oil which was purified by silica gel chromatography (hexanes/EtOAc) to afford (2S,3R)-tert-butyl 2-(tert-butoxycarbonylamino)-3-((tert-butoxycarbonylamino)methyl)-6-(4,4,5,5-tetramethyl-1,3,2-dioxaborolan-2-yl)hexanoate (Intermediate 9, 1.65 g, 71% yield) as a white foam. ¹H NMR (300 MHz, CDCl₃) δ 0.67-0.73 (2H, m), 0.95-1.16 (2H, m), 1.20 (12H, s), 1.37-1.47 (29H, m), 2.00-2.20 (1H, m), 2.36-2.56 (1H, m), 3.31-3.57 (1H, m), 4.34 (1H, br d), 5.14 (1H, br d), 5.68 (1H, br s); m/z: (ES⁻) [M+HCOO⁻]⁻=587.

### Example 1: (2S,3R)-2-amino-3-(aminomethyl)-6-boronohexanoic Acid Dihydrochloride

A solution of HBr (33 wt % in AcOH, 6.0 mL, 36 mmol) was added to a solution of (2S,3R)-tert-butyl 2-(tert-butoxycarbonylamino)-3-((tert-butoxycarbonylamino)methyl)-6-(4,4,5,5-tetramethyl-1,3,2-dioxaborolan-2-yl)hexanoate (Intermediate 9, 2.2 g, 4.1 mmol) in DCM (32 mL) and the reaction stirred at room temperature for 1 h. The reaction was diluted with Et₂O (10 mL) and concentrated. This step was repeated twice more. The residue was dissolved in Et₂O (40 mL) and 2 M aq. HCl (40 mL). Phenylboronic acid (0.989 g, 8.11 mmol) was added and the reaction was stirred at room temperature for 2 h. The layers were separated and the aqueous layer was washed with Et₂O (3×15 mL). The aqueous layer was lyophilized and purified by reverse phase chromatography (RediSep Rf Gold® C18Aq, 0 to 100% MeCN in water) to afford (2S,3R)-2-amino-3-(aminomethyl)-6-boronohexanoic acid dihydrochloride (Example 1, 0.713 g, 64% yield) as a yellow solid. ¹H NMR (300 MHz, D₂O) δ 0.65-0.87 (2H, m), 1.32-1.56 (4H, m), 2.35-2.48 (1H, m), 3.12 (2H, qd), 4.09 (1H, d); m/z: (ES⁺) [M−H₂O+H]⁺=187.

(2S,3R)-2-amino-3-(aminomethyl)-6-boronohexanoic acid dihydrochloride (Example 1, 713 mg, 2.57 mmol) was dissolved in MeOH (5 mL) and loaded onto a pre-equilibrated Porapak Rxn Cx (60 cc) ion exchange column. The resin was washed with MeOH (45 mL) followed by a 5% solution of NH₃ in MeOH (45 mL) to elute the product. Product containing fractions were collected and condensed to afford (2S,3R)-2-amino-3-(aminomethyl)-6-boronohexanoic acid (300 mg, 57% yield) as a white, powdery residue which exists as 4:1 mixture of acyclic and cyclic coordinated complex. ¹H NMR (300 MHz, D₂O) δ 0.49-0.92 (2H, m), 1.17-1.76 (4H, m), 2.03-2.18 (0.8H, m), 2.48-2.58 (0.2H, m), 3.01 (1.6H, d), 3.16-3.26 (0.4H, m), 3.49 (0.8H, d), 3.71 (0.2H, d); m/z: (ES⁺) [M−H₂O+H]⁺=187.

(2S,3R)-2-amino-3-(aminomethyl)-6-boronohexanoic acid (95 mg, 0.47 mmol) was dissolved in MeOH (3 mL) and para-toluenesulfonic acid monohydrate (266 mg, 1.40 mmol) was added. The reaction stirred at room temperature for 20 h. The reaction mixture was concentrated and directly purified by reverse phase chromatography (RediSep Rf Gold® C18Aq, 0 to 100% MeCN in water) to afford (2S,3R)-2-amino-3-(aminomethyl)-6-boronohexanoic acid ditosylate (180 mg, 71% yield) as a white solid. ¹H NMR (300 MHz, D₂O) δ 0.74-0.87 (2H, m), 1.38-1.61 (4H, m), 2.40 (6H, s), 2.45-2.48 (1H, m), 3.15 (2H, qd), 4.01 (1H, d), 7.27-7.50 (4H, m), 7.63-7.79 (4H, m); m/z: (ES⁺) [M−H₂O−2TsOH+H]⁺=187.

### Example 2: (2S,3R)-2-amino-6-borono-3-(morpholinomethyl)hexanoic Acid

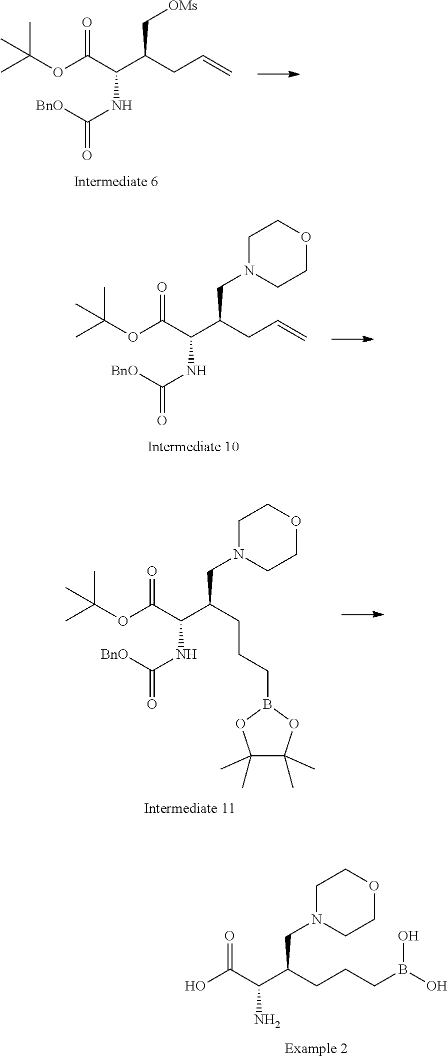 [SMILES: C=CC[C@H](COS(C)(=O)=O)[C@H](NC(=O)OCc1ccccc1)C(=O)OC(C)(C)C ; C=CC[C@H](CN1CCOCC1)[C@H](NC(=O)OCc1ccccc1)C(=O)OC(C)(C)C ; CC(C)(C)OC(=O)[C@@H](NC(=O)OCc1ccccc1)[C@H](CCCB1OC(C)(C)C(C)(C)O1)CN1CCOCC1 ; N[C@H](C(=O)O)[C@H](CCCB(O)O)CN1CCOCC1]

### Intermediate 10: (2S,3R)-tert-butyl 2-(benzyloxycarbonylamino)-3-(morpholinomethyl)hex-5-enoate

Morpholine (1.00 mL, 11.5 mmol) and potassium carbonate (829 mg, 6.00 mmol) were added to a solution of (2S,3S)-tert-butyl 2-(benzyloxycarbonylamino)-3-((methylsulfonyloxy)methyl)hex-5-enoate (Intermediate 6, 513 mg, 1.20 mmol) in DMF (5 mL) at room temperature. The reaction mixture was heated to 80° C. and stirred under an atmosphere of N₂ for 16 h. The reaction was cooled to room temperature and diluted with water (50 mL). The layers were separated and the aqueous layer was extracted with Et₂O (3×25 mL). The combined organics were dried over MgSO₄, filtered and concentrated to dryness. The crude material was purified by silica gel chromatography (hexanes/EtOAc) to afford (2S,3R)-tert-butyl 2-(benzyloxycarbonylamino)-3-(morpholinomethyl)hex-5-enoate (Intermediate 10, 285 mg, 57% yield) as a colorless oil. ¹H NMR (300 MHz, CDCl₃) δ 1.45 (9H, s), 1.63-2.61 (9H, m), 3.54-3.79 (4H, m), 4.44 (1H, br d), 4.95-5.16 (4H, m), 5.63-5.87 (1H, m), 7.05 (1H, br d), 7.25-7.39 (5H, m); m/z: (ES⁺) [M+H]⁺=419.

### Intermediate 11: (2S,3R)-tert-butyl 2-(benzyloxycarbonylamino)-3-(morpholinomethyl)-6-(4,4,5,5-tetramethyl-1,3,2-dioxaborolan-2-yl)hexanoate

Bis(1,5-cyclooctadiene)diiridium (I) dichloride (14 mg, 0.020 mmol) and bis(diphenylphosphino)methane (16 mg, 0.040 mmol) were added to an oven-dried round-bottom flask. The flask was sealed and purged with N₂. The solids were dissolved in DCM (3 mL) and 4,4,5,5-tetramethyl-1,3,2-dioxaborolane (0.22 mL, 1.5 mmol) was added slowly to the solution. The reaction stirred at room temperature for 10 min. (2S,3R)-tert-butyl 2-(benzyloxycarbonylamino)-3-(morpholinomethyl)hex-5-enoate (Intermediate 10, 285 mg, 0.680 mmol) was added to the reaction as a solution in DCM (2 mL) and the reaction mixture stirred overnight. The reaction mixture was cooled to 0° C. and quenched with MeOH (2 mL) and water (10 mL). The layers were separated and the aqueous layer was extracted with DCM (2×10 mL). The combined organics were dried over MgSO₄, filtered and concentrated to dryness. The crude material was purified by silica gel chromatography (hexanes/EtOAc) to afford (2S,3R)-tert-butyl 2-(benzyloxycarbonylamino)-3-(morpholinomethyl)-6-(4,4,5,5-tetramethyl-1,3,2-dioxaborolan-2-yl)hexanoate (Intermediate 11, 228 mg, 61% yield). ¹H NMR (300 MHz, CDCl₃) δ 0.64-0.83 (2H, br t), 1.20 (12H, s), 1.28-1.53 (13H, m), 1.93-2.63 (4H, m), 3.06-4.00 (4H, m), 4.26-4.61 (1H, m), 4.99-5.18 (2H, m), 7.26-7.39 (5H, m); m/z: (ES⁺) [M+H]⁺=547.

### Example 2: (2S,3R)-2-amino-6-borono-3-(morpholinomethyl)hexanoic Acid

(2S,3R)-tert-butyl 2-(benzyloxycarbonylamino)-3-(morpholinomethyl)-6-(4,4,5,5-tetramethyl-1,3,2-dioxaborolan-2-yl)hexanoate (Intermediate 11, 228 mg, 0.420 mmol) was dissolved in 6 M aq. HCl (4.0 mL) and the solution was heated to 100° C. for 16 h. The reaction was cooled to room temperature, diluted with water (5 mL) and washed with Et₂O (3×10 mL). The aqueous layer was lyophilized and purified by ion-exchange chromatography (PoraPak Rxn CX 20 cc column). The resin was washed with MeOH (15 mL) followed by a 5% solution of NH₃ in MeOH (15 mL) to elute the product. Product containing fractions were collected and concentrated to afford (2S,3R)-2-amino-6-borono-3-(morpholinomethyl)hexanoic acid (Example 2, 69 mg, 60% yield) as a white solid. ¹H NMR (300 MHz, D₂O) δ 0.72-0.88 (2H, m), 1.24-1.38 (1H, m), 1.40-1.58 (3H, m), 2.19-2.34 (1H, m), 2.43-2.58 (4H, m), 2.62-2.77 (2H, m), 3.71-3.85 (4H, m), 3.88 (1H, d); m/z: (ES⁺) [M+H]⁺=275.

### Example 3: (2S,3R)-2-amino-6-borono-3-(piperidin-1-ylmethyl)hexanoic Acid

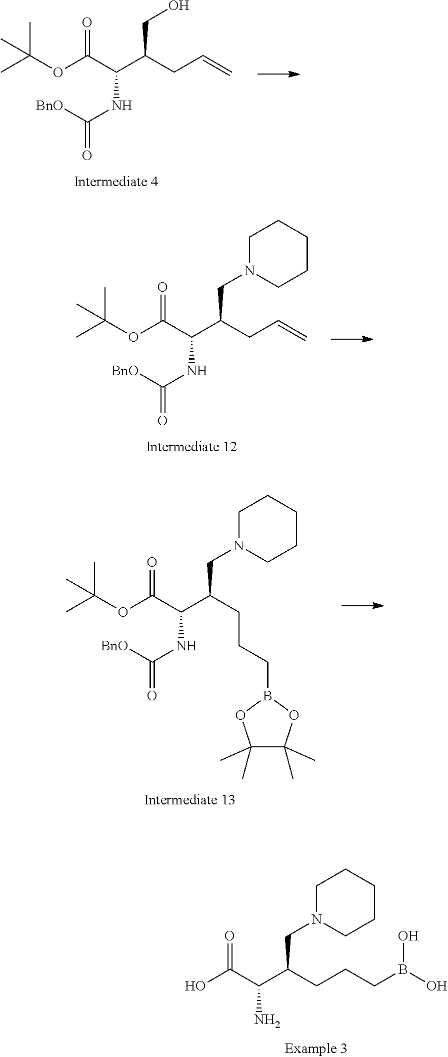 [SMILES: C=CC[C@H](CO)[C@H](NC(=O)OCc1ccccc1)C(=O)OC(C)(C)C ; C=CC[C@H](CN1CCCCC1)[C@H](NC(=O)OCc1ccccc1)C(=O)OC(C)(C)C ; CC(C)(C)OC(=O)[C@@H](NC(=O)OCc1ccccc1)[C@H](CCCB1OC(C)(C)C(C)(C)O1)CN1CCCCC1 ; N[C@H](C(=O)O)[C@H](CCCB(O)O)CN1CCCCC1]

### Intermediate 12: (2S,3R)-tert-butyl 2-(benzyloxycarbonylamino)-3-(piperidin-1-ylmethyl)hex-5-enoate

A solution of oxalyl chloride (2 M in DCM, 0.72 mL, 1.4 mmol) was added to an oven-dried flask and diluted with DCM (3 mL) and cooled to −78° C. while under an atmosphere of N₂. DMSO (0.15 mL, 2.2 mmol) was added dropwise and the reaction stirred at −78° C. for 10 min. (2S,3S)-tert-butyl 2-(benzyloxycarbonylamino)-3-(hydroxymethyl)hex-5-enoate (Intermediate 4, 250 mg, 0.72 mmol) was added slowly as a solution in DCM (3 mL) and the reaction stirred at −78° C. for 30 min. N,N-Diisopropylethylamine (0.50 mL, 2.9 mmol) was added and the reaction stirred at −78° C. for 1 h before warming to 0° C. with stirring for an additional 15 min. The reaction mixture was quenched with saturated aqueous NaHCO₃ (10 mL) and diluted with DCM (50 mL). The layers were separated and the aq. layer was extracted with DCM (2×20 mL). The combined organics were dried over anhydrous Na₂SO₄, filtered and concentrated until about 8 mL of solvent remained. The crude aldehyde was treated with piperidine (0.14 mL, 1.4 mmol), sodium triacetoxyborohydride (379 mg, 1.79 mmol) and acetic acid (0.041 mL, 0.72 mmol) and the resulting suspension stirred at room temperature for 16 h. The reaction mixture was diluted with DCM (50 mL) and saturated aqueous NaHCO₃ (10 mL) and the layers were separated. The aq. layer was extracted with DCM (2×10 mL). The combined organics were dried over Na₂SO₄, filtered and concentrated to dryness. The crude material was purified by silica gel chromatography (hexanes/EtOAc) to afford (2S,3R)-tert-butyl 2-(benzyloxycarbonylamino)-3-(piperidin-1-ylmethyl)hex-5-enoate (Intermediate 12, 269 mg, 90%) as a colorless oil. ¹H NMR (300 MHz, CDCl₃) δ ppm 1.39-1.53 (15H, m), 1.86-1.94 (1H, m), 2.07-2.46 (8H, m), 4.32 (1H, d), 5.03-5.15 (4H, m), 5.73-5.85 (1H, m), 7.26-7.35 (5H, m), 7.90 (1H, d); m/z: (ES⁺) [M+H]⁺=417.

### Intermediate 13: (2S,3R)-tert-butyl 2-(benzyloxycarbonylamino)-3-(piperidin-1-ylmethyl)-6-(4,4,5,5-tetramethyl-1,3,2-dioxaborolan-2-yl)hexanoate

Bis(1,5-cyclooctadiene)diiridium (I) dichloride (16 mg, 0.022 mmol) and bis(diphenylphosphino)methane (24 mg, 0.062 mmol) were added to an oven-dried round-bottom flask. The flask was sealed and purged with N₂. The solids were dissolved in DCM (3 mL) and 4,4,5,5-tetramethyl-1,3,2-dioxaborolane (0.20 mL, 1.4 mmol) was added slowly to the solution. The reaction stirred at room temperature for 10 min. (2S,3R)-tert-butyl 2-(benzyloxycarbonylamino)-3-(piperidin-1-ylmethyl)hex-5-enoate (Intermediate 12, 265 mg, 0.636 mmol) was added to the reaction as a solution in DCM (3 mL) and the reaction mixture stirred overnight. The reaction mixture was cooled to 0° C. and quenched with MeOH (2 mL) and water (10 mL). The layers were separated and the aqueous layer was extracted with DCM (2×10 mL). The combined organics were dried over MgSO₄, filtered and concentrated to dryness. The crude material was purified by silica gel chromatography (hexanes/EtOAc) to afford (2S,3R)-tert-butyl 2-(benzyloxycarbonylamino)-3-(piperidin-1-ylmethyl)-6-(4,4,5,5-tetramethyl-1,3,2-dioxaborolan-2-yl)hexanoate (Intermediate 13, 240 mg, 69% yield) as a colorless oil. m/z: (ES⁺) [M+H]⁺=545.

### Example 3: (2S,3R)-2-amino-6-borono-3-(piperidin-1-ylmethyl)hexanoic Acid

(2S,3R)-tert-butyl 2-(benzyloxycarbonylamino)-3-(piperidin-1-ylmethyl)-6-(4,4,5,5-tetramethyl-1,3,2-dioxaborolan-2-yl)hexanoate (Intermediate 13, 240 mg, 0.44 mmol) was dissolved in 6 M aq. HCl (12 mL) and the solution was heated to 100° C. for 16 h. The reaction mixture was cooled to room temperature, diluted with H₂O (25 mL) and washed with EtOAc (2×15 mL). The aqueous layer was concentrated under reduced pressure and the resulting residue was purified by reverse phase chromatography (RediSep Rf Gold® C18, 0 to 50% acetonitrile in water) to afford (2S,3R)-2-amino-6-borono-3-(piperidin-1-ylmethyl)hexanoic acid (Example 3, 79 mg, 36% yield) as a white solid. Obtained material was a 5.7:1 mixture of the title product and the C3 diastereomer. ¹H NMR (300 MHz, D₂O) δ 0.81-0.84 (2H, m), 1.42-1.58 (5H, m), 1.75-1.86 (3H, m), 1.93-1.99 (2H, m), 2.49 (0.2H, s, br), 2.62-2.64 (0.76H, m), 2.92-3.05 (2H, m), 3.16-3.43 (2H, m), 3.53-3.66 (2H, m), 4.15 (0.8H, d), 4.23 (0.2H, d); m/z: (ES⁺) [M+H]⁺=273.

### Example 4: (2S,3R)-2-amino-6-borono-3-((methylamino)methyl)hexanoic Acid

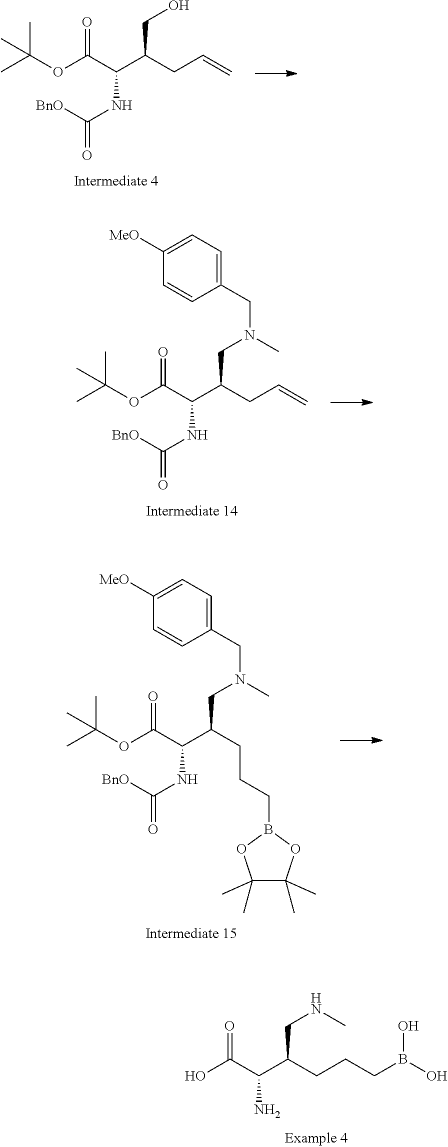 [SMILES: C=CC[C@H](CO)[C@H](NC(=O)OCc1ccccc1)C(=O)OC(C)(C)C ; C=CC[C@H](CN(C)Cc1ccc(OC)cc1)[C@H](NC(=O)OCc1ccccc1)C(=O)OC(C)(C)C ; COc1ccc(CN(C)C[C@@H](CCCB2OC(C)(C)C(C)(C)O2)[C@H](NC(=O)OCc2ccccc2)C(=O)OC(C)(C)C)cc1 ; CNC[C@@H](CCCB(O)O)[C@H](N)C(=O)O]

### Intermediate 14: (2S,3R)-tert-butyl 2-(benzyloxycarbonylamino)-3-(((4-methoxybenzyl)(methyl)amino)methyl)hex-5-enoate

A solution of oxalyl chloride (2 M in DCM, 0.57 mL, 1.1 mmol) was added to an oven-dried flask and diluted with DCM (2 mL) and cooled to −78° C. while under an atmosphere of N₂. DMSO (0.12 mL, 1.7 mmol) was added dropwise and the reaction stirred at −78° C. for 10 min. (2S,3S)-tert-butyl 2-(benzyloxycarbonylamino)-3-(hydroxymethyl)hex-5-enoate (Intermediate 4, 200 mg, 0.57 mmol) was added slowly as a solution in DCM (3 mL) and the reaction stirred at −78° C. for 30 min. N,N-Diisopropylethylamine (0.40 mL, 2.3 mmol) was added and the reaction stirred at −78° C. for 1 h before warming to 0° C. with stirring for an additional 15 min. The reaction mixture was quenched with saturated aqueous NaHCO₃ (10 mL) and diluted with DCM (20 mL). The layers were separated and the aqueous layer was extracted with DCM (2×10 mL). The combined organics were dried over anhydrous Na₂SO₄, filtered and concentrated until about 8 mL of solvent remained. The crude aldehyde was treated with 1-(4-methoxyphenyl)-N-methylmethanamine (173 mg, 1.14 mmol), sodium triacetoxyborohydride (415 mg, 1.96 mmol) and acetic acid (0.033 mL, 0.57 mmol) and the resulting suspension stirred at room temperature for 4 h. The reaction mixture was diluted with DCM (30 mL) and saturated aqueous NaHCO₃ (20 mL) and the layers were separated. The aq. layer was extracted with DCM (3×30 mL). The combined organics were dried over anhydrous Na₂SO₄, filtered and concentrated to dryness. The crude material was purified by silica gel chromatography (hexanes/EtOAc) to afford (2S,3R)-tert-butyl 2-(benzyloxycarbonylamino)-3-(((4-methoxybenzyl)(methyl)amino)methyl)hex-5-enoate (Intermediate 14, 221 mg, 80% yield) as a colorless oil. m/z: (ES⁺) [M+H]⁺=483.

### Intermediate 15: (2S,3R)-tert-butyl 2-(benzyloxycarbonylamino)-3-(((4-methoxybenzyl)(methyl)amino)methyl)-6-(4,4,5,5-tetramethyl-1,3,2-dioxaborolan-2-yl)hexanoate

Bis(1,5-cyclooctadiene)diiridium (I) dichloride (11 mg, 0.017 mmol) and bis(diphenylphosphino)methane (17 mg, 0.046 mmol) were added to an oven-dried round-bottom flask. The flask was sealed and purged with N₂. The solids were dissolved in DCM (3 mL) and 4,4,5,5-tetramethyl-1,3,2-dioxaborolane (0.15 mL, 1.0 mmol) was added slowly to the solution. The reaction stirred at room temperature for 10 min. (2S,3R)-tert-butyl 2-(benzyloxycarbonylamino)-3-(((4-methoxybenzyl)(methyl)amino)methyl)hex-5-enoate (Intermediate 14, 220 mg, 0.46 mmol) was added to the reaction as a solution in DCM (3 mL) and the reaction mixture stirred overnight. The reaction mixture was cooled to 0° C. and quenched with MeOH (2 mL) and water (10 mL). The layers were separated and the aqueous layer was extracted with DCM (2×10 mL). The combined organics were dried over MgSO₄, filtered and concentrated to dryness. The crude material was purified by silica gel chromatography (hexanes/EtOAc) to afford (2S,3R)-tert-butyl 2-(benzyloxycarbonylamino)-3-(((4-methoxybenzyl)(methyl)amino)methyl)-6-(4,4,5,5-tetramethyl-1,3,2-dioxaborolan-2-yl)hexanoate (Intermediate 15, 182 mg, 65% yield) as a colorless oil. m/z: (ES⁺) [M+H]⁺=610.

### Example 4: (2S,3R)-2-amino-6-borono-3-((methylamino)methyl)hexanoic Acid

Pd/C (10% wt, 280 mg, 0.26 mmol) was added to a solution of (2S,3R)-tert-butyl 2-(benzyloxycarbonylamino)-3-(((4-methoxybenzyl)(methyl)amino)methyl)-6-(4,4,5,5-tetramethyl-1,3,2-dioxaborolan-2-yl)hexanoate (Intermediate 15, 162 mg, 0.27 mmol) in MeOH (10 mL). The suspension was stirred under a hydrogen atmosphere (balloon, flask evacuated and backfilled with hydrogen ×3) at room temperature for 4 h. The reaction mixture was diluted with MeOH, filtered through diatomaceous earth and the filtrate was concentrated to dryness. The resulting residue was dissolved in 6 M aq. HCl (10 mL) and heated to 100° C. for 2 h. The reaction mixture was cooled to room temperature, diluted with H₂O (10 mL) and washed with DCM (2×15 mL). The aqueous layer was concentrated under reduced pressure and the resulting residue was purified by reverse phase chromatography (RediSep Rf Gold® C18, 0 to 30% acetonitrile in water) to afford (2S,3R)-2-amino-6-borono-3-((methylamino)methyl)hexanoic acid (Example 4, 33 mg, 43% yield) as a white solid. Obtained material was a 6.1:1 mixture of the title product and the C3 diastereomer. ¹H NMR (300 MHz, D₂O) δ 0.73-0.81 (2H, m), 1.35-1.55 (4H, m), 2.27-2.45 (1H, m), 2.74 (3H, s), 3.05-3.22 (1.82H, m), 3.29-3.37 (0.12H, m), 3.85-3.93 (0.81H, m), 3.99-4.03 (0.12H, m); m/z: (ES⁺) [M+H]⁺=219.

### Example 5: (2S,3R)-2-amino-6-borono-3-((dimethylamino)methyl)hexanoic Acid

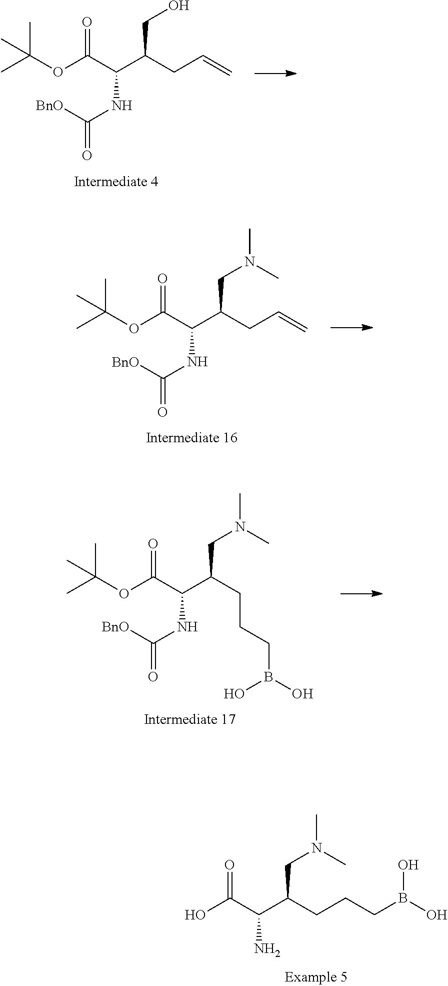 [SMILES: C=CC[C@H](CO)[C@H](NC(=O)OCc1ccccc1)C(=O)OC(C)(C)C ; C=CC[C@H](CN(C)C)[C@H](NC(=O)OCc1ccccc1)C(=O)OC(C)(C)C ; CN(C)C[C@@H](CCCB(O)O)[C@H](NC(=O)OCc1ccccc1)C(=O)OC(C)(C)C ; CN(C)C[C@@H](CCCB(O)O)[C@H](N)C(=O)O]

### Intermediate 16: (2S,3R)-tert-butyl 2-(benzyloxycarbonylamino)-3-((dimethylamino)methyl)hex-5-enoate

A solution of oxalyl chloride (2 M in DCM, 2.54 mL, 5.08 mmol) was added to an oven-dried flask and diluted with DCM (10 mL) and cooled to −78° C. while under an atmosphere of N₂. DMSO (0.54 mL, 7.6 mmol) was added dropwise and the reaction stirred at −78° C. for 10 min. (2S,3S)-tert-butyl 2-(benzyloxycarbonylamino)-3-(hydroxymethyl)hex-5-enoate (Intermediate 4, 888 mg, 2.54 mmol) was added slowly as a solution in DCM (10 mL) and the reaction stirred at −78° C. for 30 min. N,N-Diisopropylethylamine (0.50 mL, 2.9 mmol) was added and the reaction stirred at −78° C. for 1 h before warming to 0° C. with stirring for an additional 15 min. The reaction mixture was quenched with saturated aqueous NaHCO₃ (20 mL) and diluted with DCM (40 mL). The layers were separated and the aq. layer was extracted with DCM (2×20 mL). The combined organics were dried over anhydrous Na₂SO₄, filtered and concentrated to dryness. A portion of the crude aldehyde (294 mg, 0.847 mmol) was dissolved in DCM (20 mL) and a solution of dimethylamine (2 M in THF, 1.70 mL, 3.40 mmol) was added followed by sodium triacetoxyborohydride (448 mg, 2.12 mmol) and acetic acid (0.048 mL, 0.85 mmol). The resulting suspension stirred at room temperature for 15 h. The reaction mixture was diluted with DCM (50 mL) and saturated aqueous NaHCO₃ (10 mL) and the layers were separated. The aq. layer was extracted with DCM (2×10 mL). The combined organics were dried over Na₂SO₄, filtered and concentrated to dryness. The crude material was purified by silica gel chromatography (hexanes/EtOAc) to afford (2S,3R)-tert-butyl 2-(benzyloxycarbonylamino)-3-((dimethylamino)methyl)hex-5-enoate (Intermediate 16, 253 mg, 79% yield) as a colorless oil. m/z: (ES⁺) [M+H]⁺=377.

### Intermediate 17: (4R,5S)-5-(benzyloxycarbonylamino)-6-tert-butoxy-4-((dimethylamino)methyl)-6-oxohexylboronic Acid

Bis(1,5-cyclooctadiene)diiridium (I) dichloride (16 mg, 0.025 mmol) and bis(diphenylphosphino)methane (26 mg, 0.067 mmol) were added to an oven-dried round-bottom flask. The flask was sealed and purged with N₂. The solids were dissolved in DCM (3 mL) and 4,4,5,5-tetramethyl-1,3,2-dioxaborolane (0.22 mL, 1.5 mmol) was added slowly to the solution. The reaction stirred at room temperature for 10 min. (2S,3R)-tert-butyl 2-(benzyloxycarbonylamino)-3-((dimethylamino)methyl)hex-5-enoate (Intermediate 16, 253 mg, 0.672 mmol) was added to the reaction as a solution in DCM (3 mL) and the reaction mixture stirred overnight. The reaction mixture was cooled to 0° C. and quenched with MeOH (2 mL) and water (10 mL). The layers were separated and the aqueous layer was extracted with DCM (2×10 mL). The combined organics were dried over MgSO₄, filtered and concentrated to dryness. The crude material was purified by silica gel chromatography (hexanes/EtOAc) to afford (4R,5S)-5-(benzyloxycarbonylamino)-6-tert-butoxy-4-((dimethylamino)methyl)-6-oxohexylboronic acid (Intermediate 17, 223 mg, 79% yield). m/z: (ES⁺) [M+H]⁺=422.

### Example 5: (2S,3R)-2-amino-6-borono-3-((dimethylamino)methyl)hexanoic Acid

(4R,5S)-5-(benzyloxycarbonylamino)-6-tert-butoxy-4-((dimethylamino)methyl)-6-oxohexylboronic acid (Intermediate 17, 223 mg, 0.528 mmol) was dissolved in 6 M aq. HCl (15 mL) and the solution was heated to 100° C. for 20 h. The reaction mixture was cooled to room temperature and the solids were removed by filtration and washed with water. The aqueous filtrate was washed with DCM (3×20 mL) and concentrated under reduced pressure. The resulting residue was dissolved in MeOH (3 mL) and purified by ion exchange chromatography (PoraPak Rxn CX 20 cc column). The desired product was eluted from the column using a 5% ammonia in MeOH solution (20 mL). Product containing fractions were collected and concentrated to afford (2S,3R)-2-amino-6-borono-3-((dimethylamino)methyl)hexanoic acid (Example 5, 68 mg, 56% yield) as a white solid. Obtained material was a 10:1 mixture of the title product and the C3 diastereomer. ¹H NMR (300 MHz, D₂O) δ 0.77 (2H, m), 1.25-1.48 (4H, m), 2.25 (1H, m), 2.69 (6H, s), 2.85-3.05 (2H, m), 3.62 (1H, d); m/z: (ES⁺) [M+H]⁺=233.

### Example 6: (2S,3R)-2-amino-6-borono-3-(guanidinomethyl)hexanoic Acid Dihydrochloride

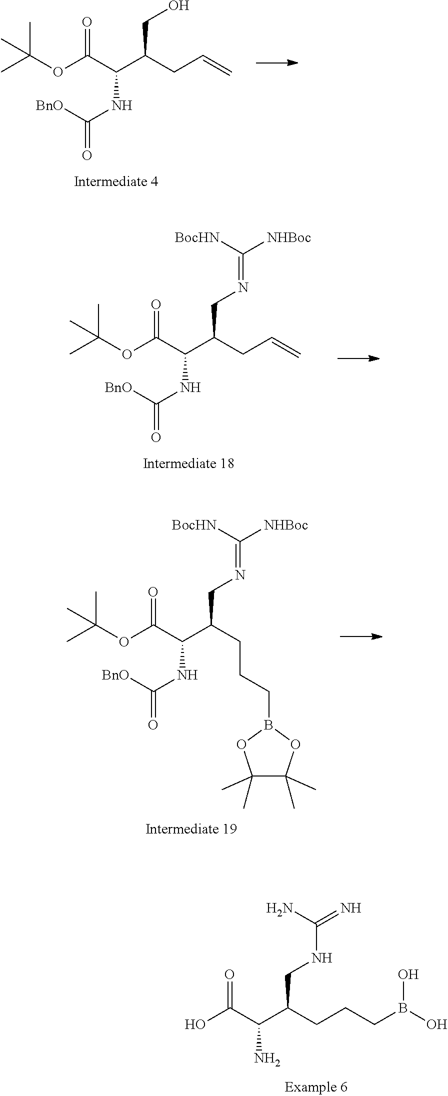 [SMILES: C=CC[C@H](CO)[C@H](NC(=O)OCc1ccccc1)C(=O)OC(C)(C)C ; C=CC[C@H](CN=C(NC(=O)OC(C)(C)C)NC(=O)OC(C)(C)C)[C@H](NC(=O)OCc1ccccc1)C(=O)OC(C)(C)C ; CC(C)(C)OC(=O)NC(=NC[C@@H](CCCB1OC(C)(C)C(C)(C)O1)[C@H](NC(=O)OCc1ccccc1)C(=O)OC(C)(C)C)NC(=O)OC(C)(C)C ; N=C(N)NC[C@@H](CCCB(O)O)[C@H](N)C(=O)O]

### Intermediate 18: tert-butyl (2S,3R)-2-(((benzyloxy)carbonyl)amino)-3-(((2,2,10,10-tetramethyl-4,8-dioxo-3,9-dioxa-5,7-diazaundecan-6-ylidene)amino)methyl)hex-5-enoate

(2S,3S)-tert-Butyl 2-(benzyloxycarbonylamino)-3-(hydroxymethyl)hex-5-enoate (Intermediate 4, 500 mg, 1.43 mmol) was dissolved in anhydrous toluene (14 mL). Triphenylphosphine (826 mg, 3.15 mmol) and 1.3-bis(tert-butoxycarbonyl)guanidine (742 mg, 2.86 mmol) were added and the solution was cooled to 0° C. DIAD (637 mg, 3.15 mmol) was added dropwise and the reaction mixture was warmed to room temperature and then further heated to 100° C. for 30 min. The reaction mixture was cooled to room temperature and washed with water (5 mL). The organic layer was dried over Na₂SO₄, filtered and concentrated to dryness. The crude material was purified by silica gel chromatography (hexanes/EtOAc) to afford tert-butyl (2S,3R)-2-(((benzyloxy)carbonyl)amino)-3-(((2,2,10,10-tetramethyl-4,8-dioxo-3,9-dioxa-5,7-diazaundecan-6-ylidene)amino)methyl)hex-5-enoate (Intermediate 18, 300 mg, 35.5%) as a sticky oil. ¹H NMR (300 MHz, CDCl₃) δ 1.40 (9H, s), 1.49-1.61 (18H, m), 2.11-2.50 (3H, m), 3.75-4.05 (2H, m), 4.21 (1H, br d), 4.97-5.17 (4H, m), 5.67-5.91 (1H, m), 6.20 (1H, br s), 7.27-7.40 (5H, m), 9.01-9.60 (2H, m); m/z: (ES⁺) [M+H]⁺=591.

### Intermediate 19: tert-butyl (2S,3R)-2-(((benzyloxy)carbonyl)amino)-6-(4,4,5,5-tetramethyl-1,3,2-dioxaborolan-2-yl)-3-(((2,2,10,10-tetramethyl-4,8-dioxo-3,9-dioxa-5,7-diazaundecan-6-ylidene)amino)methyl)hexanoate

Bis(1,5-cyclooctadiene)diiridium (I) dichloride (13 mg, 0.019 mmol) and bis(diphenylphosphino)methane (16 mg, 0.042 mmol) were added to an oven-dried round-bottom flask. The flask was sealed and purged with N₂. The solids were dissolved in DCM (8 mL) and 4,4,5,5-tetramethyl-1,3,2-dioxaborolane (0.18 mL, 1.3 mmol) was added slowly to the solution. The reaction stirred at room temperature for 10 min. tert-Butyl (2S,3R)-2-(((benzyloxy)carbonyl)amino)-3-(((2,2,10,10-tetramethyl-4,8-dioxo-3,9-dioxa-5,7-diazaundecan-6-ylidene)amino)methyl)hex-5-enoate (Intermediate 18, 300 mg, 0.51 mmol) was added to the reaction as a solution in DCM (2 mL) and the reaction stirred at room temperature for 24 h. The reaction mixture was concentrated to dryness and directly purified by silica gel chromatography (hexanes/EtOAc) to afford tert-butyl (2S,3R)-2-(((benzyloxy)carbonyl)amino)-6-(4,4,5,5-tetramethyl-1,3,2-dioxaborolan-2-yl)-3-(((2,2,10,10-tetramethyl-4,8-dioxo-3,9-dioxa-5,7-diazaundecan-6-ylidene)amino)methyl)hexanoate (Intermediate 19, 190 mg, 52% yield) as a colorless oil. ¹H NMR (300 MHz, CDCl₃) δ 0.75 (2H, t), 1.12-1.24 (14H, m), 1.33-1.42 (10H, m), 1.46 (9H, s), 1.48-1.55 (10H, m), 2.23-2.42 (1H, m), 3.54-3.87 (1H, m), 3.91-4.09 (1H, m), 4.13-4.27 (1H, m), 4.99-5.19 (2H, m), 6.12-6.50 (1H, m), 7.27-7.40 (5H, m), 9.11-9.55 (2H, m); m/z: (ES⁺) [M+H]⁺=719.

### Example 6: (2S,3R)-2-amino-6-borono-3-(guanidinomethyl)hexanoic Acid Dihydrochloride

tert-Butyl (2S,3R)-2-(((benzyloxy)carbonyl)amino)-6-(4,4,5,5-tetramethyl-1,3,2-dioxaborolan-2-yl)-3-(((2,2,10,10-tetramethyl-4,8-dioxo-3,9-dioxa-5,7-diazaundecan-6-ylidene)amino)methyl)hexanoate (Intermediate 19, 45 mg, 0.063 mmol) was dissolved in 6 M aq. HCl (2.08 mL, 12.5 mmol) and the reaction mixture was heated to 90° C. for 30 min. The reaction was cooled to 70° C. and stirred for an additional 1 h. The reaction mixture was cooled to room temperature, diluted with water (2 mL) and extracted with EtOAc (3×1 mL). The aqueous layer was lyophilized to afford (2S,3R)-2-amino-6-borono-3 (guanidinomethyl)hexanoic acid dihydrochloride (Example 6, 10 mg, 50% yield) as a white solid. ¹H NMR (300 MHz, D₂O) δ 0.80 (2H, br d), 1.37-1.62 (4H, m), 2.41 (1H, br d), 3.34 (2H, dd), 4.09 (1H, d); m/z: (ES⁺) [M+H]⁺=247.

### Example 7: (2S,3R)-2-amino-6-borono-3-(ureidomethyl)hexanoic Acid

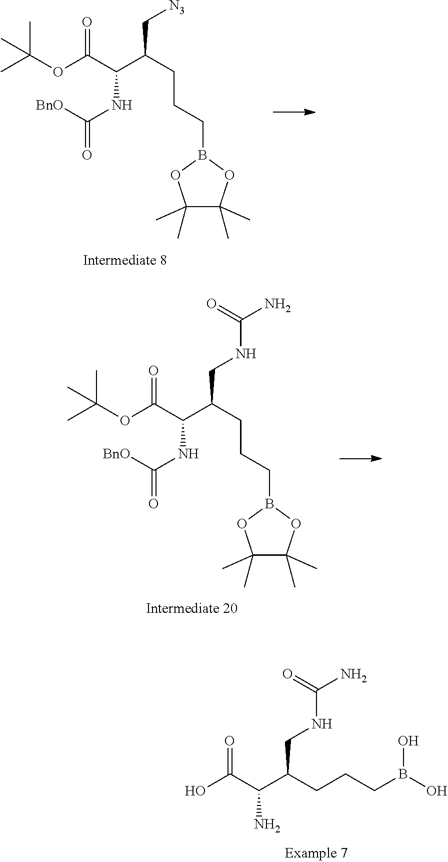 [SMILES: CC(C)(C)OC(=O)[C@@H](NC(=O)OCc1ccccc1)[C@H](CCCB1OC(C)(C)C(C)(C)O1)CN=[N+]=[N-] ; CC(C)(C)OC(=O)[C@@H](NC(=O)OCc1ccccc1)[C@H](CCCB1OC(C)(C)C(C)(C)O1)CNC(N)=O ; NC(=O)NC[C@@H](CCCB(O)O)[C@H](N)C(=O)O]

### Intermediate 20: (2S,3R)-tert-butyl 2-(benzyloxycarbonylamino)-6-(4,4,5,5-tetramethyl-1,3,2-dioxaborolan-2-yl)-3-(ureidomethyl)hexanoate

Pd/C (10% wt, 220 mg, 0.21 mmol) was added to a solution of (2S,3R)-tert-butyl 3-(azidomethyl)-2-(benzyloxycarbonylamino)-6-(4,4,5,5-tetramethyl-1,3,2-dioxaborolan-2-yl)hexanoate (Intermediate 8, 485 mg, 1.04 mmol) and isocyanatotrimethylsilane (0.35 mL, 2.6 mmol) in EtOAc (40 mL). The mixture was evacuated and backfilled with nitrogen three times and then evacuated and backfilled with hydrogen. The suspension stirred under an atmosphere of hydrogen at room temperature for 3 h. A second portion of isocyanatotrimethylsilane (0.15 mL, 1.1 mmol) was added and the suspension stirred at room temperature for an additional 20 min. The reaction mixture was diluted with DCM, filtered through diatomaceous earth and the filtrate was concentrated to dryness. The crude material was purified by silica gel chromatography (hexanes/EtOAc) to afford (2S,3R)-tert-butyl 2-(benzyloxycarbonylamino)-6-(4,4,5,5-tetramethyl-1,3,2-dioxaborolan-2-yl)-3-(ureidomethyl)hexanoate (Intermediate 20, 242 mg, 48% yield) as a white solid. m/z: (ES⁺) [M+H]⁺=486.

### Example 7: (2S,3R)-2-amino-6-borono-3-(ureidomethyl)hexanoic Acid

Trifluoroacetic acid (6.00 mL, 77.9 mmol) was added slowly to a stirred solution of (2S,3R)-tert-butyl 2-(benzyloxycarbonylamino)-6-(4,4,5,5-tetramethyl-1,3,2-dioxaborolan-2-yl)-3-(ureidomethyl)hexanoate (Intermediate 20, 242 mg, 0.466 mmol) in DCM (6 mL) and the reaction stirred at room temperature for 6 h. The solution was concentrated under reduced pressure and the resulting residue was dissolved in 1 M aq. HCl (5 mL) and Et₂O (5 mL). Phenylboronic acid (117 mg, 0.96 mmol) was added and the clear biphasic solution stirred at room temperature for 16 h. The reaction mixture was diluted with Et₂O and water and the layers were separated. The aqueous layer was washed with Et₂O (2×30 mL) and the aqueous layer was lyophilized. The crude material was purified by reverse phase chromatography (RediSep Rf Gold® C18, 0 to 50% acetonitrile in water) to afford (2S,3R)-2-amino-6-borono-3-(ureidomethyl)hexanoic acid (Example 7, 5.0 mg, 4% yield) as a white solid. ¹H NMR (300 MHz, D₂O) δ 0.77-0.82 (2H, m), 1.24-1.49 (4H, m), 2.11-2.20 (1H, m), 3.07-3.22 (2H, m), 3.71 (1H, d); m/z: (ES⁺) [M+H]⁺=248.

### Example 8: (2S,3R)-2-amino-3-(((S)-2-amino-3-methylbutanamido)methyl)-6-boronohexanoic Acid

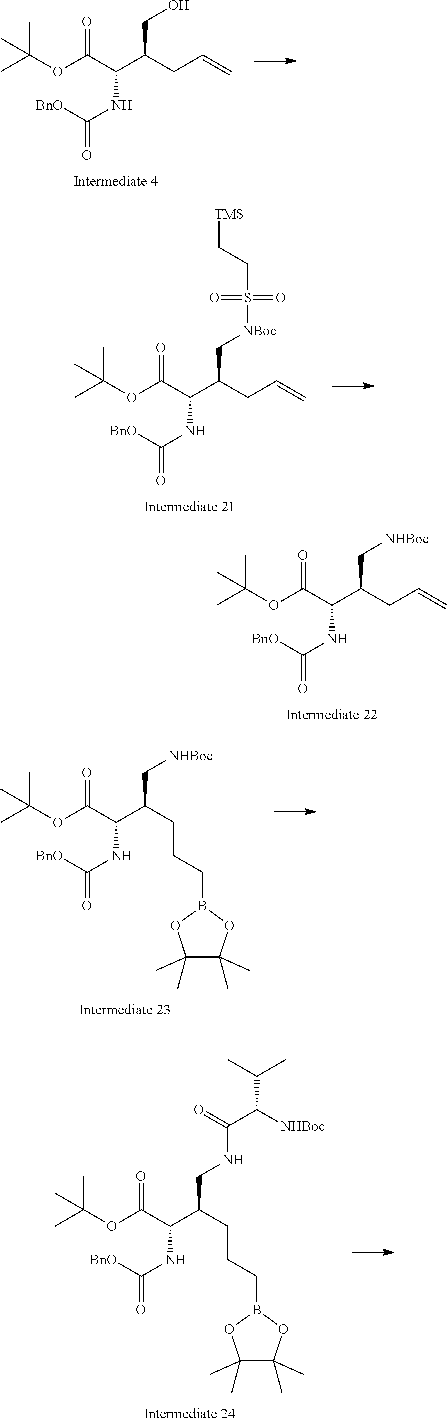 [SMILES: C=CC[C@H](CO)[C@H](NC(=O)OCc1ccccc1)C(=O)OC(C)(C)C ; C=CC[C@H](CN(C(=O)OC(C)(C)C)S(=O)(=O)CC[Si](C)(C)C)[C@H](NC(=O)OCc1ccccc1)C(=O)OC(C)(C)C ; C=CC[C@H](CNC(=O)OC(C)(C)C)[C@H](NC(=O)OCc1ccccc1)C(=O)OC(C)(C)C ; CC(C)(C)OC(=O)NC[C@@H](CCCB1OC(C)(C)C(C)(C)O1)[C@H](NC(=O)OCc1ccccc1)C(=O)OC(C)(C)C ; CC(C)[C@H](NC(=O)OC(C)(C)C)C(=O)NC[C@@H](CCCB1OC(C)(C)C(C)(C)O1)[C@H](NC(=O)OCc1ccccc1)C(=O)OC(C)(C)C]

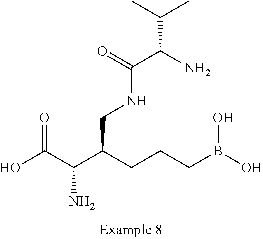 [SMILES: CC(C)[C@H](N)C(=O)NC[C@@H](CCCB(O)O)[C@H](N)C(=O)O]

### Intermediate 21: (2S,3R)-tert-butyl 2-(benzyloxycarbonylamino)-3-((N-(tert-butoxycarbonyl)-2-(trimethylsilyl)ethylsulfonamido)methyl)hex-5-enoate

(2S,3S)-tert-Butyl 2-(benzyloxycarbonylamino)-3-(hydroxymethyl)hex-5-enoate (Intermediate 4, 938 mg, 2.68 mmol) and tert-butyl ((2-(trimethylsilyl)ethyl)sulfonyl)carbamate (1.1 g, 3.9 mmol) were dissolved in THF (10 mL) and cooled to 0° C. Triphenylphosphine (1.06 mg, 4.03 mmol) and DIAD (1.1 mL, 5.7 mmol) were added and the reaction stirred for 16 h while slowly warming to room temperature. The reaction was quenched with saturated aqueous sodium bicarbonate (10 mL) and the layers were separated. The aqueous layer was extracted with EtOAc (2×10 mL). The combined organic layers were dried over MgSO₄, filtered, and concentrated to dryness. The crude material was purified by silica gel chromatography (hexanes/EtOAc) to afford (2S,3R)-tert-butyl 2-(benzyloxycarbonylamino)-3-((N-(tert-butoxycarbonyl)-2-(trimethylsilyl)ethylsulfonamido)methyl)hex-5-enoate (Intermediate 21, 1.56 g, 95%) as a colorless gum. ¹H NMR (300 MHz, CDCl₃) δ 0.04 (9H, s), 0.81-0.99 (2H, m), 1.46 (9H, s), 1.49 (9H, m), 2.05-2.29 (2H, m), 2.44-2.58 (1H, m), 3.34-3.54 (2H, m), 3.55-3.81 (2H, m), 4.35 (1H, br dd), 4.93-5.20 (4H, m), 5.42 (1H, br d), 5.69-5.96 (1H, m), 7.26-7.45 (5H, m); m/z: (ES⁺) [M+NH₄]⁺=630.

### Intermediate 22: (2S,3R)-tert-butyl 2-(benzyloxycarbonylamino)-3-((tert-butoxycarbonylamino)methyl)hex-5-enoate

(2S,3R)-tert-Butyl 2-(benzyloxycarbonylamino)-3-((N-(tert-butoxycarbonyl)-2-(trimethylsilyl)ethylsulfonamido)methyl)hex-5-enoate (Intermediate 21, 1.40 g, 2.28 mmol) was dissolved in a solution of TBAF (1 M in THF, 12.0 mL, 12.0 mmol) and the resulting solution stirred at room temperature for 16 h. The reaction was diluted with Et₂O (40 mL) and washed sequentially with water (3×25 mL) and saturated aqueous sodium bicarbonate (20 mL). The organic layer was dried with MgSO₄, filtered and concentrated to dryness. The crude material was purified by silica gel chromatography (hexanes/EtOAc) to afford (2S,3R)-tert-butyl 2-(benzyloxycarbonylamino)-3-((tert-butoxycarbonylamino)methyl)hex-5-enoate (Intermediate 22, 576 mg, 56% yield) as a colorless oil. ¹H NMR (300 MHz, CDCl₃) δ 1.43 (9H, s), 1.44 (9H, s), 1.74-1.88 (1H, m), 1.89-2.00 (1H, m), 2.17-2.30 (1H, m), 2.41-2.60 (1H, m), 3.38-3.62 (1H, m), 4.46 (1H, dd), 4.98-5.08 (2H, m), 5.09 (2H, s), 5.40 (1H, br d), 5.52-5.88 (2H, m), 7.27-7.38 (5H, m); m/z: (ES⁺) [M+H]⁺=449.

### Intermediate 23: (2S,3R)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate

Bis(1,5-cyclooctadiene)diiridium (I) dichloride (13 mg, 0.065 mmol) and bis(diphenylphosphino)methane (49 mg, 0.13 mmol) were added to an oven-dried round-bottom flask. The flask was sealed and purged with N₂. The solids were dissolved in DCM (4 mL) and 4,4,5,5-tetramethyl-1,3,2-dioxaborolane (0.50 mL, 3.5 mmol) was added slowly to the solution. The reaction stirred at room temperature for 10 min. (2S,3R)-tert-Butyl 2-(benzyloxycarbonylamino)-3-((tert-butoxycarbonylamino)methyl)hex-5-enoate (Intermediate 22, 576 mg, 1.28 mmol) was added to the reaction as a solution in DCM (3 mL) and the reaction stirred at room temperature for 3 d. The reaction mixture was cooled to 0° C. and quenched with MeOH (2 mL) and water (15 mL). The layers were separated and the aqueous layer was extracted with DCM (2×10 mL). The combined organics were dried over MgSO₄, filtered and concentrated to dryness. The crude material was purified by silica gel chromatography (hexanes/EtOAc) to afford (2S,3R)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate (Intermediate 23, 424 mg, 57% yield) as a pale yellow gum. ¹H NMR (300 MHz, CDCl₃) δ 0.57-0.81 (2H, m), 0.94-1.15 (2H, m), 1.19 (12H, s), 1.28-1.59 (20H, m), 2.05-2.23 (1H, m), 2.40-2.57 (1H, m), 3.39-3.60 (1H, m), 4.43 (1H, dd), 5.08 (2H, s), 5.41 (1H, br d), 5.53-5.78 (1H, m), 7.27-7.38 (5H, m); m/z: (ES⁺) [M+H]⁺=577.

### Intermediate 24: (2S,3R)-tert-butyl 2-(benzyloxycarbonylamino)-3-(((S)-2-(tert-butoxycarbonylamino)-3-methylbutanamido)methyl)-6-(4,4,5,5-tetramethyl-1,3,2-dioxaborolan-2-yl)hexanoate

A solution of HCl (4 M in dioxane, 1.5 mL, 6.0 mmol) was added to a solution of (2S,3R)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate (Intermediate 23, 424 mg, 0.740 mmol) in dioxane (1.5 mL) at 0° C. The reaction stirred for 6 h while slowly warming to room temperature. The solution was concentrated to afford a yellow gum which was used directly without purification. In a separate flask, HATU (619 mg, 1.63 mmol) was added to a solution of Boc-L-Val-OH (354 mg, 1.63 mmol) in DMF (3.5 mL) and the reaction stirred at room temperature for 10 min. The crude amine was dissolved in DMF (3.5 mL) and added to the second reaction flask. N,N-Diisopropylethylamine (0.45 mL, 2.6 mmol) was added and the reaction stirred at room temperature for 16 h. The reaction mixture was diluted with EtOAc (15 mL) and washed with 1 M aq HCl (60 mL) and 5% aqueous lithium chloride (10 mL). The organic layer was dried with MgSO₄, filtered and concentrated to dryness. The crude material was purified by silica gel chromatography (DCM/MeOH) to afford (2S,3R)-tert-butyl 2-(benzyloxycarbonylamino)-3-(((S)-2-(tert-butoxycarbonylamino)-3-methylbutanamido)methyl)-6-(4,4,5,5-tetramethyl-1,3,2-dioxaborolan-2-yl)hexanoate (Intermediate 24, 476 mg, 95% yield) as a pale yellow gum. ¹H NMR (300 MHz, CDCl₃) δ 0.59-0.82 (2H, m), 0.87-0.98 (6H, m), 1.18 (12H, s), 1.28-1.55 (22H, m), 2.07-2.24 (2H, m), 2.31-2.51 (1H, m), 3.38-3.54 (1H, m), 3.74-3.89 (1H, m), 3.89-4.06 (1H, m), 4.35 (1H, dd), 5.08 (2H, s), 5.50 (1H, br d), 6.92 (1H, br s), 7.26-7.37 (5H, m); m/z: (ES⁻) [M+HCOO⁻]⁻=720.

### Example 8: (2S,3R)-2-amino-3-(((S)-2-amino-3-methylbutanamido)methyl)-6-boronohexanoic Acid

(2S,3R)-tert-butyl 2-(benzyloxycarbonylamino)-3-(((S)-2-(tert-butoxycarbonylamino)-3-methylbutanamido)methyl)-6-(4,4,5,5-tetramethyl-1,3,2-dioxaborolan-2-yl)hexanoate (Intermediate 24, 476 mg, 0.700 mmol) was dissolved in a solution of HBr (33 wt % in AcOH, 3.5 mL, 21 mmol) and the reaction stirred at room temperature for 20 min. The reaction was diluted with Et₂O (10 mL) and concentrated. This step was repeated twice more. The resulting residue was dissolved in 2 M aq. HCl (5 mL) and Et₂O (5 mL). Phenylboronic acid (172 mg, 1.41 mmol) was added and the clear biphasic solution stirred at room temperature for 2 h. The layers were separated and the aqueous layer was washed with Et₂O (3×10 mL) and lyophilized. The resulting solid was dissolved in MeOH (1 mL) and purified by ion exchange chromatography (HyperSep Retain CX column). The desired product was eluted from the column using a 5% ammonia in MeOH solution (15 mL). The obtained material was further purified by reverse phase chromatography (RediSep Rf Gold® C18, 0 to 100% acetonitrile in water) to afford (2S,3R)-2-amino-3-(((S)-2-amino-3-methylbutanamido)methyl)-6-boronohexanoic acid (Example 8, 46 mg, 21% yield) as a white solid. ¹H NMR (300 MHz, D₂O) δ 0.70-0.85 (2H, m), 0.96 (6H, dd), 1.24-1.59 (4H, m), 2.00 (1H, sextet), 2.16-2.35 (1H, m), 3.23-3.43 (3H, m), 3.68 (1H, d); m/z: (ES⁻) [M−H]⁻=302.

### Example 9: (2S,3R)-2-amino-3-(((S)-2-aminobutanamido)methyl)-6-boronohexanoic Acid

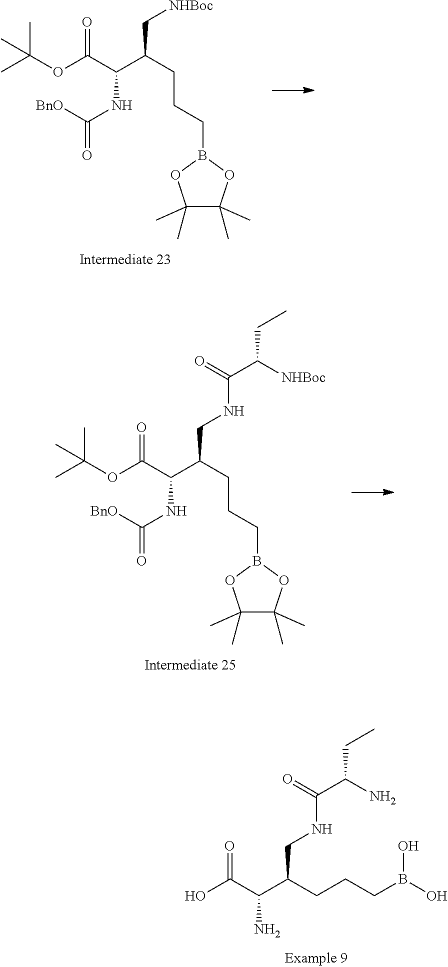 [SMILES: CC(C)(C)OC(=O)NC[C@@H](CCCB1OC(C)(C)C(C)(C)O1)[C@H](NC(=O)OCc1ccccc1)C(=O)OC(C)(C)C ; CC[C@H](NC(=O)OC(C)(C)C)C(=O)NC[C@@H](CCCB1OC(C)(C)C(C)(C)O1)[C@H](NC(=O)OCc1ccccc1)C(=O)OC(C)(C)C ; CC[C@H](N)C(=O)NC[C@@H](CCCB(O)O)[C@H](N)C(=O)O]

### Intermediate 25: (2S,3R)-tert-butyl 2-(benzyloxycarbonylamino)-3-(((S)-2-(tert-butoxycarbonylamino)butanamido)methyl)-6-(4,4,5,5-tetramethyl-1,3,2-dioxaborolan-2-yl)hexanoate

A solution of HCl (4 M in dioxane, 4.40 mL, 17.6 mmol) was added to a solution of (2S,3R)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate (Intermediate 23, 1.27 g, 2.20 mmol) in dioxane (1.5 mL) at 0° C. The reaction stirred for 4.5 h while slowly warming to room temperature. The solution was concentrated to afford a yellow gum which was used directly without purification. In a separate flask, HATU (619 mg, 1.63 mmol) was added to a solution of Boc-Abu-OH (335 mg, 1.65 mmol) in DMF (5 mL) and the reaction stirred at room temperature for 10 min. The crude amine from the previous operation was divided into two even portions (assumed 524 mg, 1.10 mmol), and one portion was dissolved in DMF (5 mL) and added to the second reaction flask. N,N-Diisopropylethylamine (0.60 mL, 3.4 mmol) was added and the reaction stirred at room temperature for 2 h. The reaction mixture was diluted with EtOAc (15 mL) and washed with 1 M aq. HCl (60 mL) and 5% aqueous lithium chloride (10 mL). The organic layer was dried over MgSO₄, filtered and concentrated to dryness. The crude material was purified by silica gel chromatography (DCM/MeOH) to afford (2S,3R)-tert-butyl 2-(benzyloxycarbonylamino)-3-(((S)-2-(tert-butoxycarbonylamino)butanamido)methyl)-6-(4,4,5,5-tetramethyl-1,3,2-dioxaborolan-2-yl)hexanoate (Intermediate 25, 516 mg, 71% yield) as a colorless gum. ¹H NMR (300 MHz, CDCl₃) δ 0.58-0.82 (2H, m), 0.93 (3H, t), 1.19 (12H, s), 1.38-1.45 (22H, m), 1.60-1.76 (1H, m), 1.77-1.97 (2H, m), 2.10-2.26 (1H, m), 2.40 (1H, dt), 3.78-3.90 (1H, m), 4.03-4.16 (1H, m), 4.34 (1H, dd), 5.08 (2H, s), 5.15-5.24 (1H, m), 5.48 (1H, br d), 7.27-7.39 (5H, m); m/z: (ES⁺) [M+H]⁺=662.

### Example 9: (2S,3R)-2-amino-3-(((S)-2-aminobutanamido)methyl)-6-boronohexanoic Acid

(2S,3R)-tert-Butyl 2-(benzyloxycarbonylamino)-3-(((S)-2-(tert-butoxycarbonylamino)butanamido)methyl)-6-(4,4,5,5-tetramethyl-1,3,2-dioxaborolan-2-yl)hexanoate (Intermediate 25, 516 mg, 0.780 mmol) was dissolved in a solution of HBr (33 wt % in AcOH, 4.0 mL, 24 mmol) and the reaction stirred at room temperature for 20 min. The reaction was diluted with Et₂O (10 mL) and concentrated. This step was repeated twice more. The resulting residue was dissolved in 2 M aq. HCl (5 mL) and Et₂O (7 mL). Phenylboronic acid (190 mg, 1.6 mmol) was added and the clear biphasic solution stirred at room temperature for 2 h. The layers were separated and the aqueous layer was washed with Et₂O (3×10 mL) and lyophilized. The resulting solid was dissolved in MeOH (1 mL) and purified by ion exchange chromatography (Silicycle SPE-R51230B-20X column). The desired product was eluted from the column using a 5% ammonia in MeOH solution (15 mL). The obtained material was further purified by reverse phase chromatography (RediSep Rf Gold® C18, 0 to 100% acetonitrile in water) to afford (2S,3R)-2-amino-3-(((S)-2-aminobutanamido)methyl)-6-boronohexanoic acid (Example 9, 101 mg, 45% yield) as a white solid. ¹H NMR (300 MHz, D₂O) δ 0.66-0.85 (2H, m), 0.92 (3H, t), 1.24-1.55 (4H, m), 1.65-1.83 (2H, m), 2.16-2.31 (1H, m), 3.24-3.39 (2H, m), 3.56 (1H, t), 3.65 (1H, d); m/z: (ES⁺) [M+H]⁺=290.

### Example 10: (2S,3R)-2-amino-3-(((S)-2-aminopropanamido)methyl)-6-boronohexanoic Acid

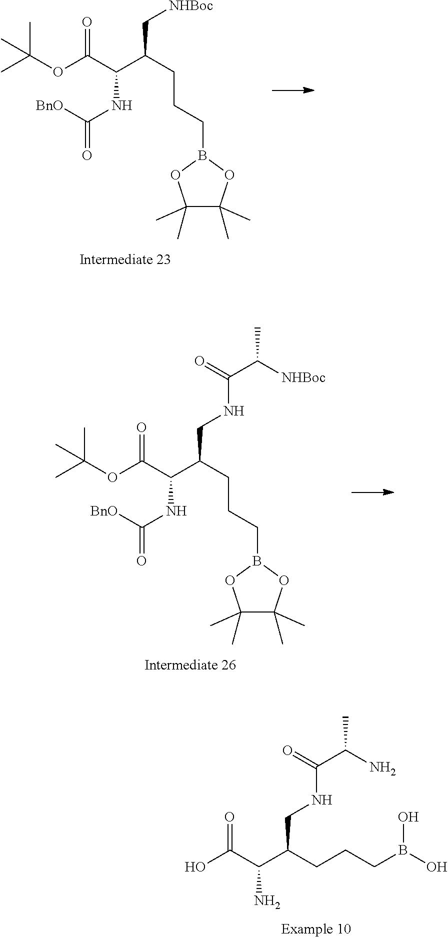 [SMILES: CC(C)(C)OC(=O)NC[C@@H](CCCB1OC(C)(C)C(C)(C)O1)[C@H](NC(=O)OCc1ccccc1)C(=O)OC(C)(C)C ; C[C@H](NC(=O)OC(C)(C)C)C(=O)NC[C@@H](CCCB1OC(C)(C)C(C)(C)O1)[C@H](NC(=O)OCc1ccccc1)C(=O)OC(C)(C)C ; C[C@H](N)C(=O)NC[C@@H](CCCB(O)O)[C@H](N)C(=O)O]

### Intermediate 26: (2S,3R)-tert-butyl 2-(benzyloxycarbonylamino)-3-(((S)-2-(tert-butoxycarbonylamino)propanamido)methyl)-6-(4,4,5,5-tetramethyl-1,3,2-dioxaborolan-2-yl)hexanoate

A solution of HCl (4 M in dioxane, 0.34 mL, 1.4 mmol) was added to a solution of (2S,3R)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate (Intermediate 23, 375 mg, 0.650 mmol) in dioxane (2 mL) at 0° C. and the reaction stirred at 0° C. for 50 min. Another portion of HCl (4 M in dioxane, 0.34 mL, 1.4 mmol) was added and the reaction stirred for an additional 1 h. An additional portion of HCl (4 M in dioxane, 0.70 mL, 2.7 mmol) was added and the reaction stirred 1 h and was then placed in a −20° C. freezer for 16 h. The reaction was allowed to warm to room temperature and stirred for 20 min. The solution was concentrated to afford a yellow gum which was used directly without purification. In a separate flask, HATU (544 mg, 1.43 mmol) was added to a solution of Boc-Ala-OH (271 mg, 1.43 mmol) in DMF (3 mL) and the reaction stirred at room temperature for 10 min. The crude amine was dissolved in DMF (3 mL) and added to the second reaction flask. N,N-Diisopropylethylamine (0.38 mL, 2.2 mmol) was added and the reaction stirred at room temperature for 2 h. The reaction mixture was diluted with EtOAc (15 mL) and washed with 1 M aq HCl (60 mL) and 5% aqueous lithium chloride (30 mL). The organic layer was dried with MgSO₄, filtered and concentrated to dryness. The crude material was purified by silica gel chromatography (DCM/MeOH) to afford (2S,3R)-tert-butyl 2-(benzyloxycarbonylamino)-3-(((S)-2-(tert-butoxycarbonylamino)propanamido)methyl)-6-(4,4,5,5-tetramethyl-1,3,2-dioxaborolan-2-yl)hexanoate (Intermediate 26, 268 mg, 64% yield) as a pale yellow gum. ¹H NMR (300 MHz, CDCl₃) δ 0.55-0.84 (2H, m), 0.96-1.27 (14H, m), 1.30-1.52 (23H, m), 2.07-2.27 (1H, m), 2.34-2.63 (1H, m), 2.74-2.99 (1H, m), 3.61-3.93 (1H, m), 3.99-4.23 (1H, m), 4.23-4.42 (1H, m), 4.90-5.24 (3H, m), 5.41-5.60 (1H, m), 7.33 (5H, br s); m/z: (ES⁺) [M+H]⁺=648.

### Example 10: (2S,3R)-2-amino-3-(((S)-2-aminopropanamido)methyl)-6-boronohexanoic Acid

(2S,3R)-tert-butyl 2-(benzyloxycarbonylamino)-3-(((S)-2-(tert-butoxycarbonylamino)propanamido)methyl)-6-(4,4,5,5-tetramethyl-1,3,2-dioxaborolan-2-yl)hexanoate (Intermediate 26, 268 mg, 0.410 mmol) was dissolved in a solution of HBr (33 wt % in AcOH, 2.0 mL, 12 mmol) and the reaction stirred at room temperature for 20 min. The reaction was diluted with Et₂O (5 mL) and concentrated. This step was repeated twice more. The resulting residue was dissolved in 2 M aq. HCl (2.5 mL) and Et₂O (5 mL). Phenylboronic acid (100 mg, 0.83 mmol) was added and the clear biphasic solution stirred at room temperature for 2 h. The layers were separated and the aqueous layer was washed with Et₂O (3×10 mL) and lyophilized. The resulting solid was dissolved in MeOH (1 mL) and purified by ion exchange chromatography (HyperSep Retain CX column). The desired product was eluted from the column using a 5% ammonia in MeOH solution (15 mL). The obtained material was further purified by reverse phase chromatography (RediSep Rf Gold® C18, 0 to 100% acetonitrile in water) to afford (2S,3R)-2-amino-3-(((S)-2-aminopropanamido)methyl)-6-boronohexanoic acid (Example 10, 45 mg, 39% yield) as a white solid. ¹H NMR (300 MHz, D₂O) δ 0.63-0.84 (2H, m), 1.20-1.55 (7H, m), 2.14-2.25 (1H, m), 3.20-3.36 (2H, m), 3.62 (1H, d), 3.74 (1H, q); m/z (ES⁺) [M+H]⁺=276.

### Example 11: (2S,3R)-3-(Acetamidomethyl)-2-amino-6-boronohexanoic Acid

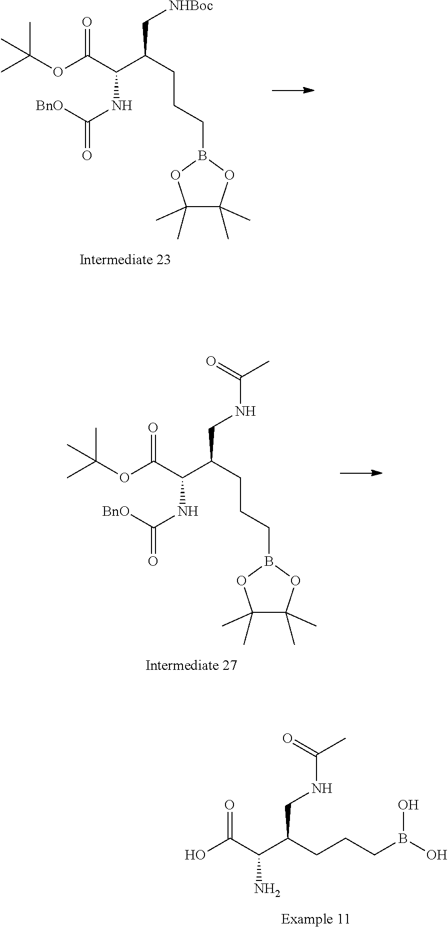 [SMILES: CC(C)(C)OC(=O)NC[C@@H](CCCB1OC(C)(C)C(C)(C)O1)[C@H](NC(=O)OCc1ccccc1)C(=O)OC(C)(C)C ; CC(=O)NC[C@@H](CCCB1OC(C)(C)C(C)(C)O1)[C@H](NC(=O)OCc1ccccc1)C(=O)OC(C)(C)C ; CC(=O)NC[C@@H](CCCB(O)O)[C@H](N)C(=O)O]

### Intermediate 27: (2S,3R)-tert-butyl 3-(acetamidomethyl)-2-(benzyloxycarbonylamino)-6-(4,4,5,5-tetramethyl-1,3,2-dioxaborolan-2-yl)hexanoate

A solution of HCl (4 M in dioxane, 4.40 mL, 17.6 mmol) was added to a solution of (2S,3R)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate (Intermediate 23, 1.27 g, 2.20 mmol) in dioxane (1.5 mL) at 0° C. The reaction stirred for 4.5 h while slowly warming to room temperature. The solution was concentrated to afford a yellow gum which was used directly without purification. The crude amine was divided into two even portions (assumed 524 mg, 1.10 mmol), and one portion was dissolved in DCM (5 mL). Triethylamine (0.40 mL, 2.9 mmol) was added and the reaction stirred at room temperature for 10 min. Acetyl chloride (0.10 mL, 1.4 mmol) was added and the reaction stirred for an additional 2 h. The reaction was quenched with water (15 mL) and diluted with DCM (20 mL). The layers were separated and the aqueous layer was with DCM (2×10 mL). The combined organic layers were washed with saturated aqueous sodium bicarbonate (20 mL), then dried over MgSO₄, filtered and concentrated to dryness. The crude material was purified by silica gel chromatography (DCM/MeOH) to afford (2S,3R)-tert-butyl 3-(acetamidomethyl)-2-(benzyloxycarbonylamino)-6-(4,4,5,5-tetramethyl-1,3,2-dioxaborolan-2-yl)hexanoate (Intermediate 27, 538 mg, 94% yield) as a colorless oil. ¹H NMR (300 MHz, CDCl₃) δ 0.55-0.81 (2H, m), 1.19 (12H, s), 1.26-1.59 (13H, m), 1.98 (3H, s), 2.07-2.20 (1H, m), 2.33-2.45 (1H, m), 3.84 (1H, ddd), 4.36 (1H, dd), 5.08 (2H, s), 5.49 (1H, br d), 6.76-6.90 (1H, m), 7.26-7.40 (5H, m); m/z: (ES⁺) [M+H]⁺=519.

### Example 11: (2S,3R)-3-(acetamidomethyl)-2-amino-6-boronohexanoic Acid

(2S,3R)-tert-butyl 3-(acetamidomethyl)-2-(benzyloxycarbonylamino)-6-(4,4,5,5-tetramethyl-1,3,2-dioxaborolan-2-yl)hexanoate (Intermediate 27, 538 mg, 1.04 mmol) was dissolved in a solution of HBr (33 wt % in AcOH, 5.0 mL, 30 mmol) and the reaction stirred at room temperature for 20 min. The reaction was diluted with Et₂O (10 mL) and concentrated. This step was repeated twice more. The resulting residue was dissolved in 2 M aq. HCl (7 mL) and Et₂O (7 mL). Phenylboronic acid (253 mg, 2.08 mmol) was added and the clear biphasic solution stirred at room temperature for 2 h. The layers were separated and the aqueous layer was washed with Et₂O (3×10 mL) and lyophilized. The resulting solid was dissolved in MeOH (1 mL) and purified by ion exchange chromatography (Silicycle SPE-R51230B-20X column). The desired product was eluted from the column using a 5% ammonia in MeOH solution (15 mL). The obtained material was further purified by reverse phase chromatography (RediSep Rf Gold® C18, 0 to 100% acetonitrile in water) to afford (2S,3R)-3-(acetamidomethyl)-2-amino-6-boronohexanoic acid (Example 11, 84 mg, 33% yield) as a white solid. ¹H NMR (300 MHz, D₂O) δ 0.67-0.86 (2H, m), 1.26-1.59 (4H, m), 2.01 (3H, s), 2.14-2.31 (1H, m), 3.15-3.39 (2H, m), 3.71 (1H, d); m/z: (ES⁺) [M+H]⁺=247.

### Example 12: (2S,3R)-3-(aminomethyl)-6-borono-2-(methylamino)hexanoic Acid

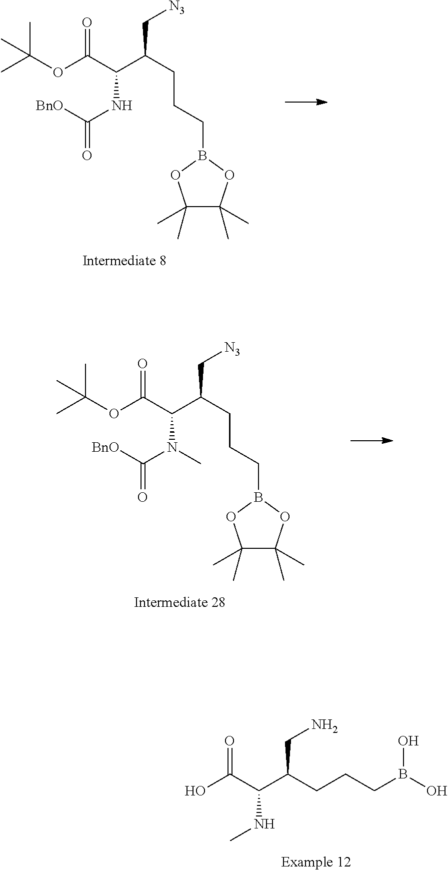 [SMILES: CC(C)(C)OC(=O)[C@@H](NC(=O)OCc1ccccc1)[C@H](CCCB1OC(C)(C)C(C)(C)O1)CN=[N+]=[N-] ; CN(C(=O)OCc1ccccc1)[C@H](C(=O)OC(C)(C)C)[C@H](CCCB1OC(C)(C)C(C)(C)O1)CN=[N+]=[N-] ; CN[C@H](C(=O)O)[C@@H](CN)CCCB(O)O]

### Intermediate 28: (2S,3R)-tert-butyl 3-(azidomethyl)-2-((benzyloxycarbonyl)(methyl)amino)-6-(4,4,5,5-tetramethyl-1,3,2-dioxaborolan-2-yl)hexanoate

(2S,3R)-tert-Butyl 3-(azidomethyl)-2-(benzyloxycarbonylamino)-6-(4,4,5,5-tetramethyl-1,3,2-dioxaborolan-2-yl)hexanoate (Intermediate 8, 155 mg, 0.310 mmol) was dissolved in DMF (2 mL) and the solution was cooled to 0° C. Sodium hydride (60% wt dispersion in oil, 15 mg, 0.37 mmol) was added and the reaction was warmed to room temperature and stirred for 15 min. Iodomethane (0.05 mL, 0.8 mmol) was added and the reaction stirred at room temperature for 16 h. The reaction mixture was diluted with water (15 mL) and extracted with Et₂O (3×15 mL). The combined organics were washed with 5% aqueous lithium chloride (10 mL), dried over MgSO₄, filtered and concentrated to dryness. The crude material was purified by silica gel chromatography (hexanes/EtOAc) to afford (2S,3R)-tert-butyl 3-(azidomethyl)-2-((benzyloxycarbonyl)(methyl)amino)-6-(4,4,5,5-tetramethyl-1,3,2-dioxaborolan-2-yl)hexanoate (Intermediate 28, 109 mg, 68% yield) as a colorless oil and as a 55:45 mixture of rotamers. ¹H NMR (300 MHz, CDCl₃) δ 0.66-0.82 (2H, m), 1.21 (12H, s), 1.23-1.37 (3H, m), 1.40 (5H, s), 1.42 (4H, s) 1.46-1.53 (1H, m), 2.06-2.22 (1H, m), 2.86 (3H, s), 3.37-3.55 (2H, m), 4.50 (0.55H, br d), 4.65 (0.45H, br d), 4.95-5.30 (2H, m), 7.26-7.41 (5H, m); m/z: (ES⁺) [M+NH₄]⁺=534.

### Example 12: (2S,3R)-3-(aminomethyl)-6-borono-2-(methylamino)hexanoic Acid

Pd/C (10% wt, 22 mg, 0.020 mmol) and di-tert-butyl-dicarbonate (115 mg, 0.530 mmol) were added to a solution of (2S,3R)-tert-butyl 3-(azidomethyl)-2-((benzyloxycarbonyl)(methyl)amino)-6-(4,4,5,5-tetramethyl-1,3,2-dioxaborolan-2-yl)hexanoate (Intermediate 28, 109 mg, 0.210 mmol) in EtOAc (2 mL). The flask was equipped with a balloon of H₂ and the suspension stirred at room temperature for 16 h. The reaction mixture was filtered through diatomaceous earth and rinsed with EtOAc. The filtrate was concentrated to dryness and then dissolved in DCM (3 mL). A solution of HBr (33% in AcOH, 0.50 mL, 3.0 mmol) was added and the reaction stirred at room temperature for 20 min. The reaction was diluted with Et₂O (5 mL) and concentrated. This step was repeated twice more. The resulting residue was dissolved in 2 M aq. HCl (2 mL) and Et₂O (2 mL). Phenylboronic acid (51 mg, 0.42 mmol) was added and the clear biphasic solution stirred at room temperature for 2 h. The layers were separated and the aqueous layer was washed with Et₂O (3×10 mL) and lyophilized. The resulting solid was purified by reverse phase chromatography (RediSep Rf Gold® C18, 0 to 100% acetonitrile in water) to afford a dark yellow solid. Obtained material was redissolved in MeOH (1 mL) and loaded onto a pre-equilibrated Hypersep Retain CX (2 g) ion exchange column. The resin was washed with MeOH (15 mL) followed by a 5% solution of NH₃ in MeOH (15 mL) to elute the product. Product containing fractions were concentrated to a colorless residue which was further purified by reverse phase chromatography (RediSep Rf Gold® C18Aq, 0 to 100% MeCN in water) to afford (2S,3R)-3-(aminomethyl)-6-borono-2-(methylamino)hexanoic acid (Example 12, 15 mg, 33%) as a white solid. ¹H NMR (300 MHz, D₂O) δ 0.68-0.91 (2H, m), 1.34-1.59 (4H, m), 2.11-2.26 (1H, m), 2.60 (3H, s), 3.07 (2H, qd), 3.51 (1H, d); m/z: (ES⁺) [M−H₂O+H]⁺=201.

### Example 13: (2S,3R)-2-((S)-2-Amino-3-methylbutanamido)-3-(aminomethyl)-6-boronohexanoic Acid

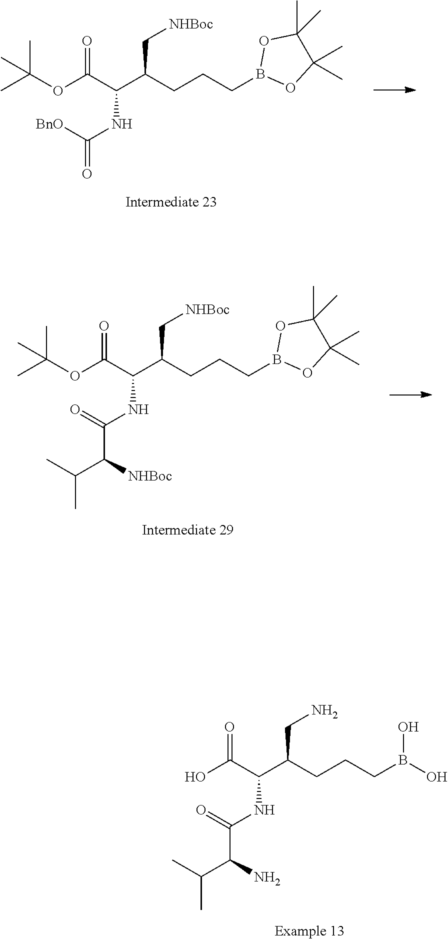

### Intermediate 29: (2S,3R)-tert-butyl 2-((S)-2-(tert-butoxycarbonylamino)-3-methylbutanamido)-3-((tert-butoxycarbonylamino)methyl)-6-(4,4,5,5-tetramethyl-1,3,2-dioxaborolan-2-yl)hexanoate

Pd/C (10% wt, 180 mg, 0.16 mmol) was added to a solution of (2S,3R)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate (Intermediate 23, 950 mg, 1.65 mmol) in EtOAc (12 mL). The flask was equipped with a balloon of H₂ and the suspension stirred at room temperature for 16 h. The reaction mixture was filtered through diatomaceous earth and rinsed with EtOAc (50 mL). The filtrate was concentrated to afford a pale yellow oil, which was carried on directly without further purification. In a separate flask, HATU (363 mg, 0.95 mmol) was added to a solution of Boc-Val-OH (210 mg, 0.95 mmol) in DMF (4 mL) and the reaction stirred at room temperature for 10 min. The crude amine from the previous operation was divided into two even portions (assumed 364 mg, 0.825 mmol), and one portion was dissolved in DMF (5 mL) and added to the second reaction flask. N,N-Diisopropylethylamine (0.35 mL, 2.0 mmol) was added and the reaction stirred at room temperature for 2 h. The reaction mixture was diluted with EtOAc (15 mL) and washed with 1 M aq. HCl (80 mL) and saturated aqueous sodium chloride (20 mL). The organic layer was dried over MgSO₄, filtered and concentrated to dryness. The crude material was purified by silica gel chromatography (DCM/MeOH) to afford (2S,3R)-tert-butyl 2-((S)-2-(tert-butoxycarbonylamino)-3-methylbutanamido)-3-((tert-butoxycarbonylamino)methyl)-6-(4,4,5,5-tetramethyl-1,3,2-dioxaborolan-2-yl)hexanoate (Intermediate 29, 400 mg, 72% yield) as a colorless gum. ¹H NMR (300 MHz, DMSO-d6) δ 0.55-0.69 (2H, m), 0.84 (6H, dd), 1.16 (12H, s), 1.31-1.43 (31H, m), 1.83-2.07 (2H, m), 2.55-2.66 (1H, m), 2.98-3.13 (1H, m), 3.83 (1H, br t), 4.30-4.47 (1H, m), 6.29-6.44 (1H, m), 6.72 (1H, br d), 7.83-7.94 (1H, m); m/z: (ES⁻) [M+HCOO⁻]⁻=686.

### Example 13: (2S,3R)-2-((S)-2-amino-3-methylbutanamido)-3-(aminomethyl)-6-boronohexanoic Acid

A solution of HBr (33 wt % in AcOH, 0.75 mL, 4.6 mmol) was added to a solution of (2S,3R)-tert-butyl 2-((S)-2-(tert-butoxycarbonylamino)-3-methylbutanamido)-3-((tert-butoxycarbonylamino)methyl)-6-(4,4,5,5-tetramethyl-1,3,2-dioxaborolan-2-yl)hexanoate (Intermediate 29, 200 mg, 0.31 mmol) in DCM (4 mL) and the reaction stirred at room temperature for 1 h. The reaction was diluted with Et₂O (5 mL) and concentrated. This step was repeated twice more. The resulting residue was dissolved in 1 M aq. HCl (7 mL) and Et₂O (5 mL). Phenylboronic acid (114 mg, 0.940 mmol) was added and the clear biphasic solution stirred at room temperature for 2 h. The layers were separated and the aqueous layer was washed with Et₂O (3×10 mL) and lyophilized. The resulting solid was dissolved in MeOH (1 mL) and purified by ion exchange chromatography (PoraPak Rxn CX 20 cc column). The desired product was eluted from the column using a 5% ammonia in MeOH solution (15 mL). The obtained material was further purified by reverse phase chromatography (RediSep Rf Gold® C18, 0 to 100% acetonitrile in water) to afford (2S,3R)-2-((S)-2-amino-3-methylbutanamido)-3-(aminomethyl)-6-boronohexanoic acid (Example 13, 57 mg, 61% yield) as a white solid. ¹H NMR (300 MHz, D₂O) δ 0.67-0.85 (2H, m), 0.97 (6H, dd), 1.16-1.56 (4H, m), 1.95-2.15 (1H, sextet), 2.38 (1H, td), 2.76 (1H, dd), 3.14 (1H, dd), 3.47 (1H, d), 4.41 (1H, d); m/z: (ES⁺) [M+H]⁺=304.

### Example 14: (2S,3R)-3-(aminomethyl)-2-((S)-2-aminopropanamido)-6-boronohexanoic Acid

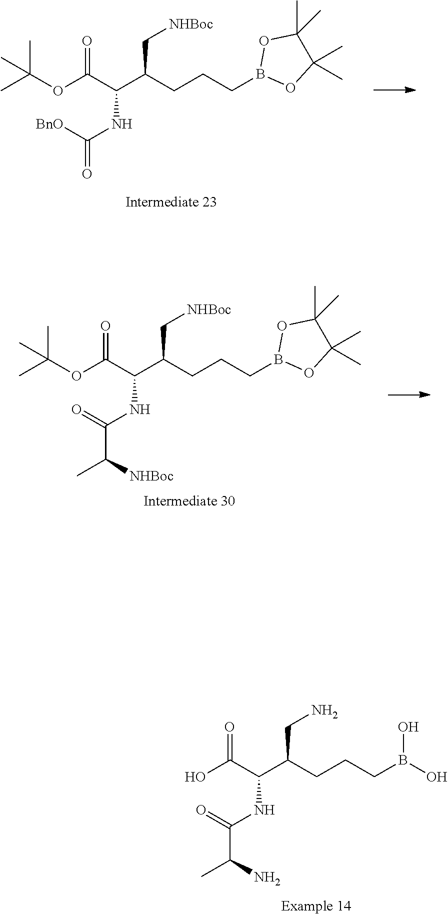

### Intermediate 23 Intermediate 30 Example 14 Intermediate 30: (2S,3R)-tert-butyl 3-((tert-butoxycarbonylamino)methyl)-2-((S)-2-(tert-butoxycarbonylamino)propanamido)-6-(4,4,5,5-tetramethyl-1,3,2-dioxaborolan-2-yl)hexanoate

Pd/C (10% wt, 180 mg, 0.16 mmol) was added to a solution of (2S,3R)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate (Intermediate 23, 950 mg, 1.65 mmol) in EtOAc (12 mL). The flask was equipped with a balloon of H₂ and the suspension stirred at room temperature for 16 h. The reaction mixture was filtered through diatomaceous earth and rinsed with EtOAc (50 mL). The filtrate was concentrated to afford a pale yellow oil, which was carried on directly without further purification. In a separate flask, HATU (363 mg, 0.95 mmol) was added to a solution of Boc-Ala-OH (180 mg, 0.95 mmol) in DMF (4 mL) and the reaction stirred at room temperature for 10 min. The crude amine from the previous operation was divided into two even portions (assumed 364 mg, 0.825 mmol), and one portion was dissolved in DMF (5 mL) and added to the second reaction flask. N,N-Diisopropylethylamine (0.35 mL, 2.0 mmol) was added and the reaction stirred at room temperature for 16 h. The reaction mixture was diluted with EtOAc (15 mL) and washed with 1 M aq HCl (80 mL) and saturated aqueous sodium chloride (20 mL). The organic layer was dried over MgSO₄, filtered and concentrated to dryness. The crude material was purified by silica gel chromatography (DCM/MeOH) to afford (2S,3R)-tert-butyl 3-((tert-butoxycarbonylamino)methyl)-2-((S)-2-(tert-butoxycarbonylamino)propanamido)-6-(4,4,5,5-tetramethyl-1,3,2-dioxaborolan-2-yl)hexanoate (Intermediate 30, 191 mg, 36% yield) as a colorless oil. ¹H NMR (300 MHz, CDCl₃) δ 0.54-0.82 (2H, m), 0.84-1.12 (2H, m), 1.19 (12H, s), 1.29-1.38 (4H, m), 1.38-1.58 (28H, m), 2.07-2.24 (1H, m), 2.25-2.44 (1H, m), 3.29-3.61 (1H, m), 4.05-4.19 (1H, m), 4.55-4.71 (1H, m), 4.86-5.14 (1H, m), 5.60-6.01 (1H, m), 6.66 (1H, br d); m/z: (ES⁺) [M+H]⁺=614.

### Example 14: (2S,3R)-3-(aminomethyl)-2-((S)-2-aminopropanamido)-6-boronohexanoic Acid

A solution of HBr (33 wt % in AcOH, 0.75 mL, 4.6 mmol) was added to a solution of (2S,3R)-tert-butyl 3-((tert-butoxycarbonylamino)methyl)-2-((S)-2-(tert-butoxycarbonylamino)propanamido)-6-(4,4,5,5-tetramethyl-1,3,2-dioxaborolan-2-yl)hexanoate (Intermediate 30, 191 mg, 0.310 mmol) in DCM (4 mL) and the reaction stirred at room temperature for 1.5 h. The reaction was diluted with Et₂O (5 mL) and concentrated. This step was repeated twice more. The resulting residue was dissolved in 1 M aq. HCl (7 mL) and Et₂O (7 mL). Phenylboronic acid (114 mg, 0.940 mmol) was added and the clear biphasic solution stirred at room temperature for 2 h. The layers were separated and the aqueous layer was washed with Et₂O (3×5 mL) and lyophilized. The resulting solid was dissolved in MeOH (1 mL) and purified by ion exchange chromatography (PoraPak Rxn CX 20 cc column). The desired product was eluted from the column using a 5% ammonia in MeOH solution (15 mL). The obtained material was further purified by reverse phase chromatography (RediSep Rf Gold® C18, 0 to 100% acetonitrile in water) to afford (2S,3R)-3-(aminomethyl)-2-((S)-2-aminopropanamido)-6-boronohexanoic acid (Example 14, 59 mg, 68% yield) as a white solid. ¹H NMR (300 MHz, D₂O) δ 0.67-0.87 (2H, m), 1.17-1.55 (7H, m), 2.39 (1H, tq), 2.74 (1H, dd), 3.12 (1H, dd), 3.79 (1H, q), 4.40 (1H, d); m/z: (ES⁺) [M+H]⁺=276.

### Example 15: (2S,3R)-2-amino-3-(2-aminoethyl)-6-boronohexanoic Acid Dihydrochloride

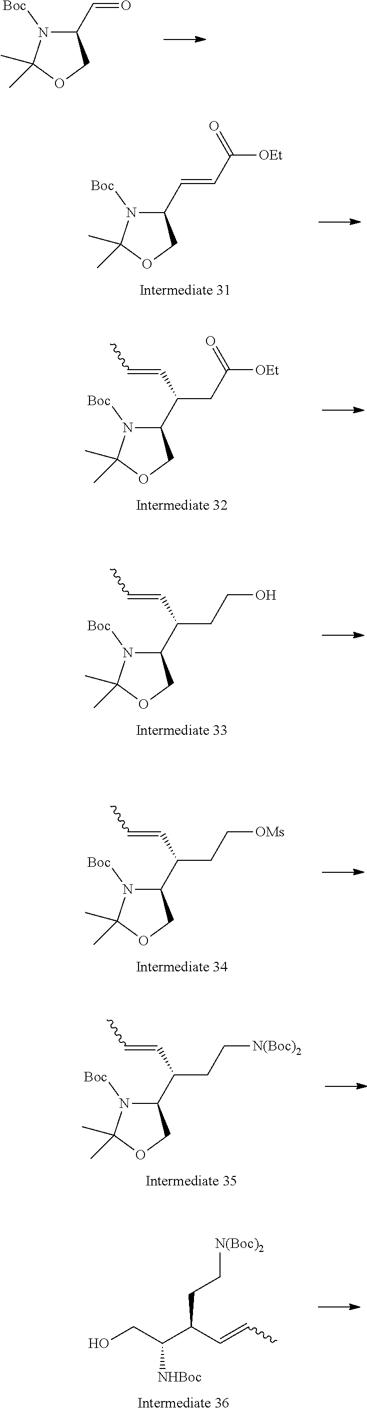

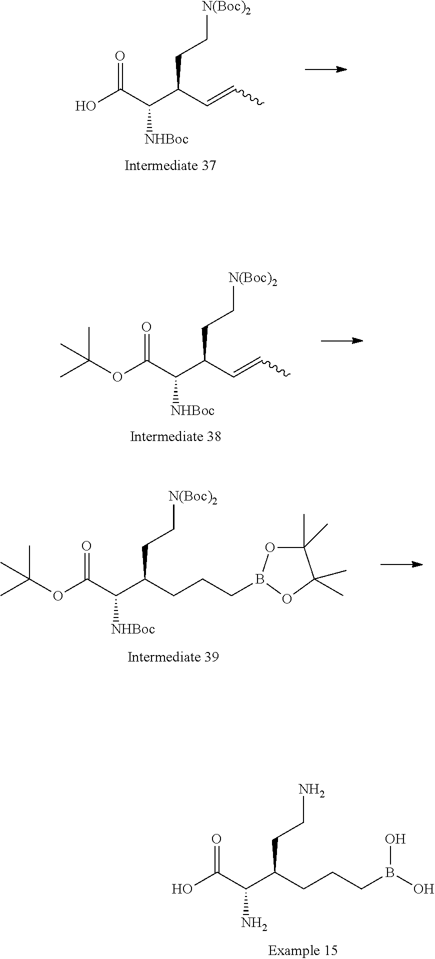

### Intermediate 31: (S,E)-tert-butyl 4-(3-ethoxy-3-oxoprop-1-enyl)-2,2-dimethyloxazolidine-3-carboxylate

Methyl (triphenylphosphoranylidene)acetate (9.62 g, 28.8 mmol) was add to a solution of tert-butyl (R)-4-formyl-2,2-dimethyloxazolidine-3-carboxylate (6.00 g, 26.2 mmol) in toluene (220 mL) at 0° C. After addition, the reaction was warmed to room temperature and stirred for 40 h. The reaction mixture was concentrated and the resulting residue was diluted with Et₂O (50 mL). The solids were removed by filtration and washed with Et₂O (20 mL). The filtrate was concentrated to dryness. The crude material was purified by silica gel chromatography (hexanes/EtOAc) to afford (S,E)-tert-butyl 4-(3-ethoxy-3-oxoprop-1-enyl)-2,2-dimethyloxazolidine-3-carboxylate (Intermediate 31, 5.74 g, 77% yield) as a mixture of rotamers. ¹H NMR (300 MHz, CDCl₃) δ 1.37-1.50 (9H, m), 1.51-1.57 (3H, m), 1.63 (3H, br s), 3.70-3.83 (4H, m), 4.05-4.12 (1H, m), 4.33-4.64 (1H, m), 5.84-6.03 (1H, m), 6.85 (1H, br dd); m/z: (ES⁺) [M+H]⁺=286.

### Intermediate 32: (S)-tert-butyl 4-((S)-1-ethoxy-1-oxohex-4-en-3-yl)-2,2-dimethyloxazolidine-3-carboxylate

A 3-neck flask was dried under N₂. Copper (1) iodide (23.4 g, 123 mmol) was added and the solids were diluted in THF (100 mL). The suspension was cooled to 5° C. and a solution of prop-1-en-1-ylmagnesium bromide (0.5 M in THF, 491 mL, 245 mmol) was added dropwise via an additional funnel under an atmosphere of N₂. The mixture was stirred at 5° C. for 30 min and then cooled to −78° C. After stirring at −78° C. for 10 min, trimethylsilyl chloride (15.68 mL, 122.7 mmol) was added followed by a solution of (S,E)-tert-butyl 4-(3-ethoxy-3-oxoprop-1-enyl)-2,2-dimethyloxazolidine-3-carboxylate (Intermediate 31, 7.00 g, 24.5 mmol) in THF (20 mL). After addition, the reaction was stirred at −78° C. for 30 min and then warmed to −30° C. to −20° C. with stirring for an additional 30 min. The reaction was carefully quenched with concentrated aq. NH₄OH: saturated aq. NH₄Cl (1:9, 200 mL) at −20° C. The crude mixture was warmed to room temperature and the solids were removed by vacuum filtration. The biphasic filtrate was separated and the aqueous phase was extracted with EtOAc (3×100 mL). The combined organics were dried over Na₂SO₄, filtered and concentrated to dryness. The crude material was purified by silica gel chromatography (hexanes/EtOAc) to afford (S)-tert-butyl 4-((S)-1-ethoxy-1-oxohex-4-en-3-yl)-2,2-dimethyloxazolidine-3-carboxylate (Intermediate 32, 6.80 g, 85% yield) as a mixture of rotamers and E/Z olefins. ¹H NMR (300 MHz, CDCl₃) δ 1.40-1.52 (12H, m), 1.54-1.75 (6H, m), 2.14-2.36 (1H, m), 2.42-2.65 (1H, m), 3.29-3.55 (1H, m), 3.63 (3H, m), 3.73-4.01 (3H, m), 5.10-5.37 (1H, m), 5.46-5.70 (1H, m); m/z: (ES⁺) [M+H]⁺=328.

### Intermediate 33: (S)-tert-butyl 4-((S)-1-hydroxyhex-4-en-3-VI)-2,2-dimethyloxazolidine-3-carboxylate

(S)-tert-Butyl 4-((S)-1-ethoxy-1-oxohex-4-en-3-yl)-2,2-dimethyloxazolidine-3-carboxylate (Intermediate 32, 3.50 g, 10.7 mmol) was dissolved in anhydrous THF (18 mL) and the solution was cooled to 0° C. under an atmosphere of N₂. A solution of LAH (2 M in THF, 5.34 mL, 10.7 mmol) was added dropwise to the reaction and the reaction mixture stirred at 0° C. for 1 h. The reaction was carefully quenched with water (0.4 mL), 15% aq. NaOH (0.4 mL) and water (1.2 mL) at 0° C. The resulting suspension was warmed to room temperature and stirred for 10 min. Na₂SO₄ (5 g) was added and the suspension was filtered and the solid cake was washed with EtOAc (50 mL). The filtrate was concentrated to dryness. The crude product was purified by silica gel chromatography (hexanes/EtOAc) to afford (S)-tert-butyl 4-((S)-1-hydroxyhex-4-en-3-yl)-2,2-dimethyloxazolidine-3-carboxylate (Intermediate 33, 3.10 g, 97% yield) as a mixture of rotamers and E/Z olefins. ¹H NMR (300 MHz, CDCl₃) δ 1.43-1.52 (14H, m), 1.54-1.72 (6H, m), 1.73-1.88 (1H, m), 2.48-3.17 (1H, m), 3.50-3.73 (2H, m), 3.75-4.01 (3H, m), 5.12-5.38 (1H, m), 5.43-5.77 (1H, m); m/z: (ES⁺) [M+H]⁺=300.

### Intermediate 34: (S)-tert-butyl 2,2-dimethyl-4-((S)-1-(methylsulfonyloxy)hex-4-en-3-yl)oxazolidine-3-carboxylate

Triethylamine (1.96 mL, 14.0 mmol) and methanesulfonic anhydride (2.27 g, 13.0 mmol) were added sequentially to a solution of (S)-tert-butyl 4-((S)-1-hydroxyhex-4-en-3-yl)-2,2-dimethyloxazolidine-3-carboxylate (Intermediate 33, 3.00 g, 10.0 mmol) in DCM (25 mL) at 0° C. The reaction was warmed to room temperature and stirred for 1 h. The crude reaction mixture was diluted with DCM (25 ml) and washed with 1 M aq. HCl (10 mL) and saturated NaHCO₃ (5 mL). The organics were dried over Na₂SO₄, filtered and concentrated to dryness to afford (S)-tert-butyl 2,2-dimethyl-4-((S)-1-(methylsulfonyloxy)hex-4-en-3-yl)oxazolidine-3-carboxylate (Intermediate 34, as a mixture of rotamers and E/Z olefins which was used without further purification. ¹H NMR (300 MHz, CDCl₃) δ 1.39-1.51 (12H, m), 1.53-1.64 (4H, m), 1.66-1.77 (3H, m), 1.89-2.12 (1H, m), 2.97 (3H, s), 3.07-3.20 (1H, m), 3.74-4.00 (3H, m), 4.03-4.17 (1H, m), 4.22-4.35 (1H, m), 5.05-5.27 (1H, m), 5.54-5.84 (m, 1H); m/z: (ES⁺) [M+H]⁺=378.

### Intermediate 35: (S)-tert-butyl 4-((S)-1-(bis(tert-butoxycarbonyl)amino)hex-4-en-3-yl)-2,2-dimethyloxazolidine-3-carboxylate

Sodium hydride (60% wt in oil, 427 mg, 10.68 mmol) was added to a solution of di-tert-butyl iminodicarboxylate (2.319 g, 10.68 mmol) in DMF (30 mL) at 0° C. and the suspension stirred at 0° C. for 20 min before warming to room temperature with stirring for an additional 10 min. A solution of (S)-tert-butyl 2,2-dimethyl-4-((S)-1-(methylsulfonyloxy)hex-4-en-3-yl)oxazolidine-3-carboxylate (Intermediate 34, 2.60 g, 6.89 mmol) in DMF (3 mL) was added and the reaction was heated to 95° C. for 3 h under an atmosphere of N₂. The reaction mixture was cooled to room temperature and concentrated. The resulting residue was diluted in water (10 mL) and EtOAc (20 mL) and the layers were separated. The organic phase was dried over Na₂SO₄, filtered and concentrated to dryness. The crude material was purified by silica gel chromatography (hexanes/EtOAc) to afford (S)-tert-butyl 4-((S)-1-(bis(tert-butoxycarbonyl)amino)hex-4-en-3-yl)-2,2-dimethyloxazolidine-3-carboxylate (Intermediate 35, 2.60 g, 76% yield). ¹H NMR (300 MHz, CDCl₃) δ 1.36-1.52 (30H, m), 1.53-1.95 (8H, m), 2.26-2.97 (1H, m), 3.28-3.44 (1H, m), 3.48-3.66 (1H, m), 3.70-4.00 (3H, m), 5.14-5.29 (1H, m), 5.40-5.83 (1H, m); m/z: (ES⁺) [M+Na]⁺=521.

### Intermediate 36: tert-butyl (tert-butoxycarbonyl)((S)-3-((S)-1-((tert-butoxycarbonyl)amino)-2-hydroxyethyl)hex-4-en-1-yl)carbamate

Cerium (III) chloride heptahydrate (3.36 g, 9.02 mmol) and oxalic acid (0.027 g, 0.30 mmol) were added sequentially to a solution of (S)-tert-butyl 4-((S)-1-(bis(tert-butoxycarbonyl)amino)hex-4-en-3-yl)-2,2-dimethyloxazolidine-3-carboxylate (Intermediate 35, 1.50 g, 3.01 mmol) in acetonitrile (30 mL) at room temperature. The resulting suspension stirred at room temperature for 2 h. The suspension was filtered and solids were washed with EtOAc. The filtrate was concentrated and then diluted with EtOAc (50 mL) and washed with water (20 mL) and brine (10 mL). The organic layer was dried over Na₂SO₄, filtered and concentrated to dryness. The crude material was purified by silica gel chromatography (hexanes/EtOAc) to afford tert-butyl (tert-butoxycarbonyl)((S)-3-((S)-1-((tert-butoxycarbonyl)amino)-2-hydroxyethyl)hex-4-en-1-yl)carbamate (Intermediate 36, 830 mg, 60% yield). ¹H NMR (300 MHz, CDCl₃) δ 1.43 (9H, s), 1.49 (18H, s), 1.56-1.85 (5H, m), 2.17-2.77 (1H, m), 3.34-3.72 (5H, m), 4.52-4.76 (1H, m), 5.13-5.35 (1H, m), 5.47-5.78 (1H, m); m/z: (ES⁺) [M+H]⁺=459.

### Intermediate 37: (2S,3S)-3-(2-(bis(tert-butoxycarbonyl)amino)ethyl)-2-(tert-butoxycarbonylamino)hex-4-enoic Acid

tert-Butyl (tert-butoxycarbonyl)((S)-3-((S)-1-((tert-butoxycarbonyl)amino)-2-hydroxyethyl)hex-4-en-1-yl)carbamate (Intermediate 36, 400 mg, 0.87 mmol) was dissolved in acetone (5 mL) and cooled to −20° C. under an atmosphere of N₂. Jones reagent (2.37 M in aq H₂SO₄, 1.14 mL, 3.05 mmol) was slowly added and the solution stirred at −20 to −10° C. for 5 h. The reaction mixture was concentrated and the residue was partitioned between water (10 mL) and EtOAc (10 mL). The aqueous phase was extracted with EtOAc (3×10 mL). The combined organics were dried over Na₂SO₄, filtered and concentrated to dryness. The crude material was purified by silica gel chromatography (hexanes/EtOAc) to afford (2S,3S)-3-(2-(bis(tert-butoxycarbonyl)amino)ethyl)-2-(tert-butoxycarbonylamino)hex-4-enoic acid (Intermediate 37, 200 mg, 48% yield) which was contaminated with impurities. m/z: (ES⁺) [M+H]⁺=471.

### Intermediate 38: (2S,3S)-tert-butyl 3-(2-(bis(tert-butoxycarbonyl)amino)ethyl)-2-(tert-butoxycarbonylamino)hex-4-enoate

2-tert-Butyl-1,3-diisopropylisourea (0.42 mL, 1.9 mmol) was added to a solution of (2S,3S)-3-(2-(bis(tert-butoxycarbonyl)amino)ethyl)-2-(tert-butoxycarbonylamino)hex-4-enoic acid (Intermediate 37, 250 mg, 0.53 mmol) in DCM (5 mL) and the reaction stirred at room temperature under an atmosphere of N₂ for 16 h. The reaction suspension was filtered to remove the insoluble solids. 2-tert-Butyl-1,3-diisopropylisourea (0.05 mL, 0.2 mmol) was added to the filtrate and the reaction stirred at room temperature for an additional 48 h. The crude mixture was concentrated to dryness and directly purified by silica gel chromatography (hexanes/EtOAc) to afford (2S,3S)-tert-butyl 3-(2-(bis(tert-butoxycarbonyl)amino)ethyl)-2-(tert-butoxycarbonylamino)hex-4-enoate (Intermediate 38, 120 mg, 43% yield) as a sticky oil. ¹H NMR (300 MHz, CDCl₃) δ 1.33-1.57 (37H, m), 1.60-1.68 (3H, m), 1.71-1.90 (1H, m), 2.45-3.06 (1H, m), 3.37-3.66 (2H, m), 4.20-4.28 (1H, m), 4.90-5.08 (1H, m), 5.18 (1H, td), 5.44-5.87 (1H, m); m/z: (ES⁺) [M+Na]⁺=551.

### Intermediate 39: (2S,3R)-tert-butyl 3-(2-(bis(tert-butoxycarbonyl)amino)ethyl)-2-(tert-butoxycarbonylamino)-6-(4,4,5,5-tetramethyl-1,3,2-dioxaborolan-2-yl)hexanoate

Bis(1,5-cyclooctadiene)diiridium (I) dichloride (15 mg, 0.022 mmol) and bis(diphenylphosphino)methane (17 mg, 0.046 mmol) were added to an oven-dried round-bottom flask. The flask was sealed and purged with N₂. The solids were dissolved in DCM (2.6 mL) and 4,4,5,5-tetramethyl-1,3,2-dioxaborolane (0.21 mL, 1.4 mmol) was added slowly to the solution. The reaction stirred at room temperature for 10 min. (2S,3S)-tert-butyl 3-(2-(bis(tert-butoxycarbonyl)amino)ethyl)-2-(tert-butoxycarbonylamino)hex-4-enoate (Intermediate 38, 300 mg, 0.57 mmol) was added to the reaction as a solution in DCM (2 mL) and the reaction stirred at room temperature for 24 h. The reaction mixture was concentrated to dryness and directly purified by silica gel chromatography (hexanes/EtOAc) to afford (2S,3R)-tert-butyl 3-(2-(bis(tert-butoxycarbonyl)amino)ethyl)-2-(tert-butoxycarbonylamino)-6-(4,4,5,5-tetramethyl-1,3,2-dioxaborolan-2-yl)hexanoate (Intermediate 39, 100 mg, 27% yield) as a colorless oil. ¹H NMR (300 MHz, CDCl₃) δ 0.73 (2H, t), 1.22 (12H, s), 1.43 (12H, s), 1.46-1.74 (30H, m), 1.80-1.90 (1H, br d), 3.42-3.77 (2H, m), 4.28 (1H, br dd), 5.03 (1H, br d); m/z: (ES⁺) [M+Na]⁺=679.

### Example 15: (2S,3R)-2-amino-3-(2-aminoethyl)-6-boronohexanoic Acid Dihydrochloride

(2S,3R)-tert-Butyl 3-(2-(bis(tert-butoxycarbonyl)amino)ethyl)-2-(tert-butoxycarbonylamino)-6-(4,4,5,5-tetramethyl-1,3,2-dioxaborolan-2-yl)hexanoate (Intermediate 39, 230 mg, 0.35 mmol) was dissolved in HCl (4 M in dioxane, 1.75 mL, 7.01 mmol) and the reaction stirred at room temperature under an atmosphere of N₂ for 30 min. The reaction was heated to 60° C. and stirred for 1 h. The reaction was cooled to room temperature and diluted with 1 M aq. HCl (1 mL). Phenylboronic acid (214 mg, 1.75 mmol) was added and the reaction was heated to 60° C. for 1 h. The reaction mixture was cooled to room temperature and the volatiles were removed in vacuo. The crude solution was diluted with water (5 mL) and washed with EtOAc (4×3 mL). The aqueous phase was lyophilized to afford (2S,3R)-2-amino-3-(2-aminoethyl)-6-boronohexanoic acid dihydrochloride (Example 15, 80 mg, 78% yield) as a dry film. ¹H NMR (300 MHz, D₂O) δ 0.81 (2H, brt), 1.35-1.55 (4H, m), 1.72-1.91 (2H, m), 2.06-2.30 (1H, m), 3.05-3.23 (2H, m), 4.07 (1H, d); m/z: (ES⁺) [M+H]⁺=219.

### Example 16: (2S,3S)-2-amino-6-borono-3-carbamoylhexanoic Acid

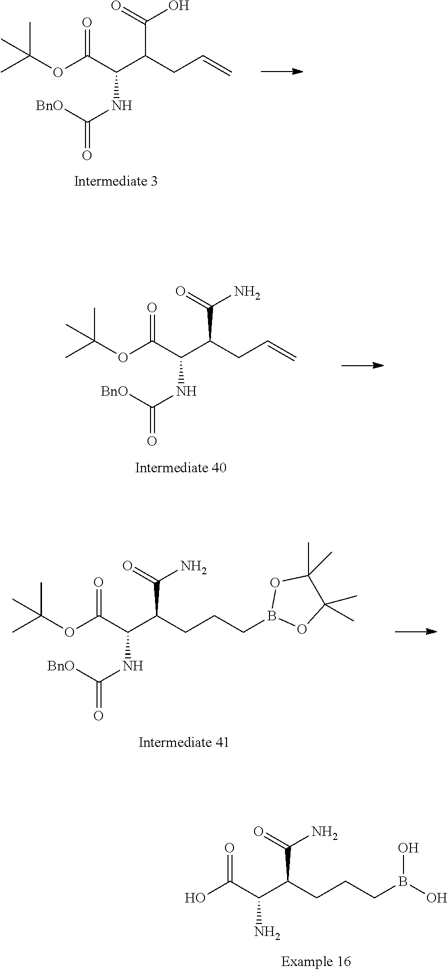 [SMILES: C=CCC(C(=O)O)[C@H](NC(=O)OCc1ccccc1)C(=O)OC(C)(C)C ; C=CC[C@H](C(N)=O)[C@H](NC(=O)OCc1ccccc1)C(=O)OC(C)(C)C ; CC(C)(C)OC(=O)[C@@H](NC(=O)OCc1ccccc1)[C@H](CCCB1OC(C)(C)C(C)(C)O1)C(N)=O ; NC(=O)[C@@H](CCCB(O)O)[C@H](N)C(=O)O]

### Intermediate 40: (2S,3S)-tert-butyl 2-(benzyloxycarbonylamino)-3-carbamoylhex-5-enoate

HATU (311 mg, 0.818 mmol), ammonium chloride (159 mg, 2.97 mmol) and N,N-diisopropylethylamine (0.78 mL, 4.5 mmol) were added to a solution of 2-((S)-1-(benzyloxycarbonylamino)-2-tert-butoxy-2-oxoethyl)pent-4-enoic acid (Intermediate 3, 270 mg, 0.74 mmol) in DMF (3 mL) and the reaction stirred at room temperature for 15 h. The mixture was diluted with DCM and saturated aqueous ammonium chloride. The layers were separated and the aqueous layer was extracted with DCM. The combined organics were dried over Na₂SO₄, filtered and concentrated to dryness. The crude material was purified by silica gel chromatography (hexanes/EtOAc) to afford pure diastereomers (2S,3S)-tert-butyl 2-(benzyloxycarbonylamino)-3-carbamoylhex-5-enoate (Intermediate 40, 148 mg, 55% yield) and (2S,3R)-tert-butyl 2-(benzyloxycarbonylamino)-3-carbamoylhex-5-enoate (86 mg, 32% yield). The stereochemistry of the major diastereomer was assigned by analogy to previous analogues. ¹H NMR (500 MHz, CDCl₃) δ 1.39 (9H, s), 2.26-2.47 (2H, m), 2.78-3.00 (1H, m), 4.19-4.43 (1H, m), 5.01-5.16 (4H, m), 5.71-5.81 (1H, m), 5.82-5.87 (1H, m), 5.96-6.05 (1H, m), 6.13-6.21 (1H, m), 7.25-7.37 (5H, m); m/z: (ES⁺) [M+H]⁺=363.

### Intermediate 41: (2S,3S)-tert-butyl 2-(benzyloxycarbonylamino)-3-carbamoyl-6-(4,4,5,5-tetramethyl-1,3,2-dioxaborolan-2-yl)hexanoate

Bis(1,5-cyclooctadiene)diiridium (I) dichloride (15 mg, 0.022 mmol) and bis(diphenylphosphino)ethane (18 mg, 0.045 mmol) were added to an oven-dried round-bottom flask. The flask was sealed and purged with N₂. The solids were dissolved in DCM (1.5 mL) and 4,4,5,5-tetramethyl-1,3,2-dioxaborolane (67 μL, 0.46 mmol) was added slowly to the solution. The reaction stirred at room temperature for 10 min. (2S,3S)-tert-Butyl 2-(benzyloxycarbonylamino)-3-carbamoylhex-5-enoate (Intermediate 40, 84 mg, 0.23 mmol) was added to the reaction as a solution in DCM (1 mL) and the reaction stirred at room temperature for 15 h. The reaction mixture was cooled to 0° C. and quenched with MeOH (2 mL) and water (10 mL). The layers were separated and the aqueous layer was extracted with DCM (2×10 mL). The combined organics were dried over MgSO₄, filtered and concentrated to dryness. The crude material was purified by silica gel chromatography (hexanes/EtOAc) to afford (2S,3S)-tert-butyl 2-(benzyloxycarbonylamino)-3-carbamoyl-6-(4,4,5,5-tetramethyl-1,3,2-dioxaborolan-2-yl)hexanoate (Intermediate 41, 73 mg, 64% yield). ¹H NMR (400 MHz, CDCl₃) δ 0.72-0.82 (2H, m), 1.20 (12H, s), 1.34-1.41 (9H, m), 1.43-1.52 (2H, m), 1.53-1.62 (1H, m), 1.62-1.72 (1H, m), 2.68-2.93 (1H, m), 4.19-4.40 (1H, m), 5.01-5.09 (1H, m), 5.09-5.16 (1H, m), 5.67-5.94 (2H, m), 6.04-6.25 (1H, m), 7.24-7.35 (5H, m); m/z: (ES⁺) [M+H]⁺=491.

### Example 16: (2S,3S)-2-amino-6-borono-3-carbamoylhexanoic Acid

A solution of HBr (33 wt % in AcOH, 0.5 mL, 2.8 mmol) was added to a solution of (2S,3S)-tert-butyl 2-(benzyloxycarbonylamino)-3-carbamoyl-6-(4,4,5,5-tetramethyl-1,3,2-dioxaborolan-2-yl)hexanoate (Intermediate 41, 73 mg, 0.15 mmol) in DCM (2 mL) and the reaction stirred at room temperature for 1 h. The reaction was concentrated and the resulting residue was diluted in Et₂O (2 mL) and 2 M aq. HCl (2 mL). Phenylboronic acid (36 mg, 0.30 mmol) was added and the clear biphasic solution stirred at room temperature for 15 h. The layers were separated and the aqueous layer was washed with Et₂O (3×10 mL) and lyophilized. The resulting solid was dissolved in MeOH (1 mL) and purified by ion exchange chromatography (PoraPak Rxn CX 20 cc column). The desired product was eluted from the column using a 5% ammonia in MeOH solution (15 mL) to afford (2S,3S)-2-amino-6-borono-3-carbamoylhexanoic acid (Example 16, 31 mg, 96% yield) as a white solid. ¹H NMR (500 MHz, D₂O) δ 0.79 (2H, td), 1.44 (2H, quin), 1.53-1.61 (1H, m), 1.64-1.73 (1H, m), 2.87-3.00 (1H, m), 3.77 (1H, d); m/z: (ES⁺) [M+H]⁺=219.

### Example 17: (2S,3S)-2-amino-6-borono-3-(methylcarbamoyl)hexanoic Acid

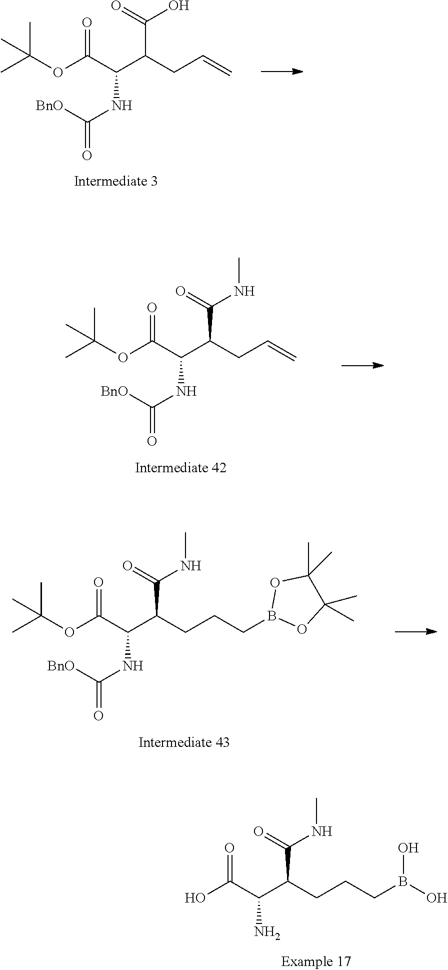 [SMILES: C=CCC(C(=O)O)[C@H](NC(=O)OCc1ccccc1)C(=O)OC(C)(C)C ; C=CC[C@H](C(=O)NC)[C@H](NC(=O)OCc1ccccc1)C(=O)OC(C)(C)C ; CNC(=O)[C@@H](CCCB1OC(C)(C)C(C)(C)O1)[C@H](NC(=O)OCc1ccccc1)C(=O)OC(C)(C)C ; CNC(=O)[C@@H](CCCB(O)O)[C@H](N)C(=O)O]

### Intermediate 42: (2S,3S)-tert-butyl 2-(benzyloxycarbonylamino)-3-(methylcarbamoyl)hex-5-enoate

HATU (266 mg, 0.699 mmol), methylamine hydrochloride (172 mg, 2.54 mmol) and N,N-diisopropylethylamine (0.67 mL, 3.8 mmol) were added to a solution of 2-((S)-1-(benzyloxycarbonylamino)-2-tert-butoxy-2-oxoethyl)pent-4-enoic acid (Intermediate 3, 231 mg, 0.64 mmol) in DMF (3 mL) and the reaction stirred at room temperature for 15 h. The mixture was diluted with DCM and saturated aqueous ammonium chloride. The layers were separated and the aqueous layer was extracted with DCM. The combined organics were dried over Na₂SO₄, filtered and concentrated to dryness. The crude material was purified by silica gel chromatography (hexanes/EtOAc) to afford pure diastereomers (2S,3S)-tert-butyl 2-(benzyloxycarbonylamino)-3-(methylcarbamoyl)hex-5-enoate (Intermediate 42, 133 mg, 56% yield) and (2S,3R)-tert-butyl 2-(benzyloxycarbonylamino)-3-(methylcarbamoyl)hex-5-enoate (77 mg, 32% yield). The stereochemistry of the major diastereomer was assigned by analogy to previous analogues. ¹H NMR (500 MHz, CDCl₃) δ 1.37 (9H, s), 2.24-2.45 (2H, m), 2.71 (3H, d), 2.81 (1H, td), 4.19-4.41 (1H, m), 4.96-5.18 (4H, m), 5.66-5.77 (1H, m), 5.77-5.84 (1H, m), 6.09-6.35 (1H, m), 7.24-7.36 (5H, m); m/z: (ES⁺) [M+H]⁺=377.

### Intermediate 43: (2S,3S)-tert-butyl 2-(benzyloxycarbonylamino)-3-(methylcarbamoyl)-6-(4,4,5,5-tetramethyl-1,3,2-dioxaborolan-2-yl)hexanoate

Bis(1,5-cyclooctadiene)diiridium (I) dichloride (8.1 mg, 0.012 mmol) and bis(diphenylphosphino)ethane (9.7 mg, 0.024 mmol) were added to an oven-dried round-bottom flask. The flask was sealed and purged with N₂. The solids were dissolved in DCM (1 mL) and 4,4,5,5-tetramethyl-1,3,2-dioxaborolane (59 μL, 0.40 mmol) was added slowly to the solution. The reaction stirred at room temperature for 10 min. (2S,3S)-tert-butyl 2-(benzyloxycarbonylamino)-3-(methylcarbamoyl)hex-5-enoate (Intermediate 42, 76 mg, 0.20 mmol) was added to the reaction as a solution in DCM (1.5 mL) and the reaction stirred at room temperature for 15 h. The reaction mixture was cooled to 0° C. and quenched with MeOH (2 mL) and water (10 mL). The layers were separated and the aqueous layer was extracted with DCM (2×10 mL). The combined organics were dried over MgSO₄, filtered and concentrated to dryness. The crude material was purified by silica gel chromatography (hexanes/EtOAc) to afford (2S,3S)-tert-butyl 2-(benzyloxycarbonylamino)-3-(methylcarbamoyl)-6-(4,4,5,5-tetramethyl-1,3,2-dioxaborolan-2-yl)hexanoate (Intermediate 43, 70 mg, 69% yield). ¹H NMR (500 MHz, CDCl₃) δ 0.64-0.87 (2H, m), 1.20 (13H, s), 1.31-1.45 (10H, m), 1.49-1.60 (1H, m), 1.63-1.72 (1H, m), 2.60-2.77 (4H, m), 4.30 (1H, br dd), 5.02-5.09 (1H, m), 5.09-5.16 (1H, m), 5.75-5.91 (1H, m), 6.28 (1H, br d), 7.24-7.35 (5H, m); m/z: (ES⁺) [M+H]⁺=505.

### Example 17: (2S,3S)-2-amino-6-borono-3-(methylcarbamoyl)hexanoic Acid

A solution of HBr (33 wt % in AcOH, 0.5 mL, 2.8 mmol) was added to a solution of (2S,3S)-tert-butyl 2-(benzyloxycarbonylamino)-3-(methylcarbamoyl)-6-(4,4,5,5-tetramethyl-1,3,2-dioxaborolan-2-yl)hexanoate (Intermediate 43, 70 mg, 0.14 mmol) in DCM (2 mL) and the reaction stirred at room temperature for 1 h. The reaction was concentrated and the resulting residue was diluted in Et₂O (2 mL) and 2 M aq. HCl (2 mL). Phenylboronic acid (34 mg, 0.28 mmol) was added and the clear biphasic solution stirred at room temperature for 15 h. The layers were separated and the aqueous layer was washed with Et₂O (3×10 mL) and lyophilized. The resulting solid was dissolved in MeOH (1 mL) and purified by ion exchange chromatography (PoraPak Rxn CX 20 cc column). The desired product was eluted from the column using a 5% ammonia in MeOH solution (15 mL) to afford (2S,3S)-2-amino-6-borono-3-(methylcarbamoyl)hexanoic acid (Example 17, 30 mg, 93% yield) as a white solid. ¹H NMR (500 MHz, D₂O) δ 0.71-0.85 (2H, m), 1.31-1.45 (2H, m), 1.51-1.60 (1H, m), 1.62-1.73 (1H, m), 2.67 (3H, s), 2.89 (1H, dt), 3.80 (1H, d); m/z: (ES⁺) [M+H]⁺=233.

### Example 18: (2S,3S)-2-amino-3-(aminomethyl)-6-boronohexanoic Acid Dihydrochloride

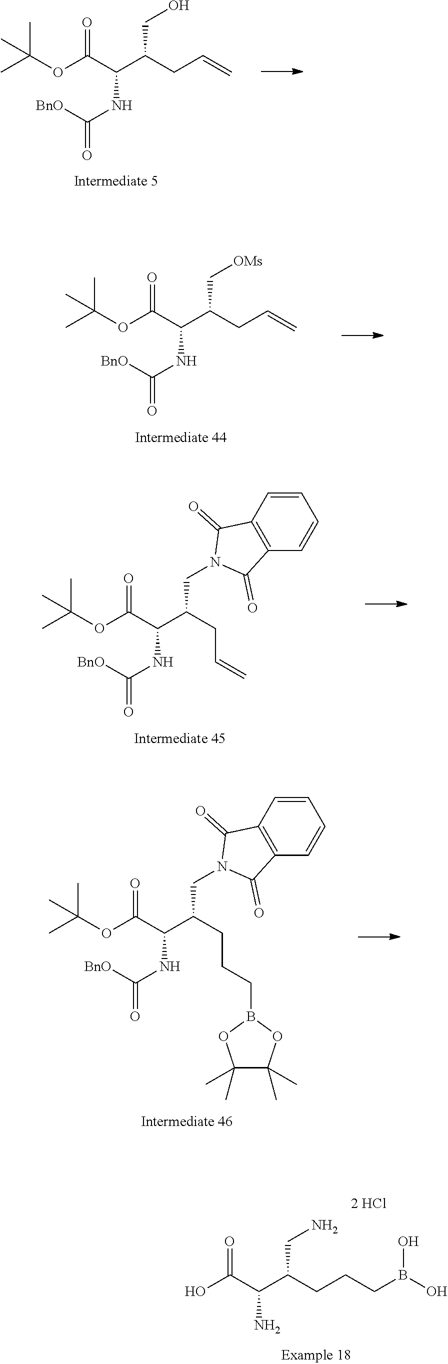 [SMILES: C=CC[C@@H](CO)[C@H](NC(=O)OCc1ccccc1)C(=O)OC(C)(C)C ; C=CC[C@@H](COS(C)(=O)=O)[C@H](NC(=O)OCc1ccccc1)C(=O)OC(C)(C)C ; C=CC[C@@H](CN1C(=O)c2ccccc2C1=O)[C@H](NC(=O)OCc1ccccc1)C(=O)OC(C)(C)C ; CC(C)(C)OC(=O)[C@@H](NC(=O)OCc1ccccc1)[C@@H](CCCB1OC(C)(C)C(C)(C)O1)CN1C(=O)c2ccccc2C1=O ; NC[C@H](CCCB(O)O)[C@H](N)C(=O)O]

### Intermediate 44: (2S,3R)-tert-butyl 2-(benzyloxycarbonylamino)-3-((methylsulfonyloxy)methyl)hex-5-enoate

Triethylamine (1.70 mL, 12.2 mmol) and methanesulfonyl chloride (0.60 mL, 7.7 mmol) were added to a solution of (2S,3R)-tert-butyl 2-(benzyloxycarbonylamino)-3-(hydroxymethyl)hex-5-enoate (Intermediate 5, 1.00 g, 2.86 mmol) in DCM (20 mL) at 0° C. The reaction was warmed to room temperature and stirred for 90 min. The crude mixture was diluted with DCM (10 mL) and washed sequentially with saturated aqueous sodium bicarbonate, water, and brine (25 mL each). The organic layer was dried over MgSO₄, filtered and concentrated to dryness. The crude material was purified by silica gel chromatography (hexanes/EtOAc) to afford (2S,3R)-tert-butyl 2-(benzyloxycarbonylamino)-3-((methylsulfonyloxy)methyl)hex-5-enoate (Intermediate 44, 1.17 g, 96% yield) as a pale yellow oil. ¹H NMR (300 MHz, CDCl₃) δ 1.46 (9H, s), 1.94-2.11 (1H, m), 2.18-2.32 (1H, m), 2.34-2.53 (1H, m), 2.97 (3H, s), 4.12-4.24 (2H, m), 4.45 (1H, br dd), 5.00-5.18 (4H, m), 5.42 (1H, br d), 5.63-5.89 (1H, m), 7.25-7.37 (5H, m); m/z: (ES⁺) [M+NH₄]⁺=445.

### Intermediate 45: (2S,3S)-tert-butyl 2-(benzyloxycarbonylamino)-3-((1,3-dioxoisoindolin-2-yl)methyl)hex-5-enoate

Potassium phthalimide (0.558 g, 3.01 mmol) and potassium iodide (0.227 g, 1.37 mmol) were added to an oven-dried flask under an atmosphere of N₂. (2S,3R)-tert-Butyl 2-(benzyloxycarbonylamino)-3-((methylsulfonyloxy)methyl)hex-5-enoate (Intermediate 44, 1.17 g, 2.74 mmol) was added as a solution in DMF (15 mL) and the reaction was heated to 95° C. for 3 h. The reaction mixture was cooled to room temperature and diluted with water (30 mL). The layers were separated and the aq. layer was extracted with Et₂O (3×20 mL). The combined organics were dried over MgSO₄, filtered and concentrated to dryness. The crude material was purified by silica gel chromatography (hexanes/EtAOc) to afford (2S,3S)-tert-butyl 2-(benzyloxycarbonylamino)-3-((1,3-dioxoisoindolin-2-yl)methyl)hex-5-enoate (Intermediate 45, 0.725 g, 55% yield) as a colorless oil. ¹H NMR (300 MHz, CDCl₃) δ 1.20 (9H, s), 2.04-2.26 (2H, m), 2.69 (1H, pentet), 3.61 (2H, d), 4.47 (1H, br d), 4.98-5.22 (4H, m), 5.75-5.94 (1H, m), 5.99 (1H, br d), 7.25-7.42 (5H, m), 7.63-7.72 (2H, m), 7.77-7.87 (2H, m); m/z: (ES⁺) [M+NH₄]⁺=496.

### Intermediate 46: (2S,3S)-tert-butyl 2-(benzyloxycarbonylamino)-3-((1,3-dioxoisoindolin-2-yl)methyl)-6-(4,4,5,5-tetramethyl-1,3,2-dioxaborolan-2-yl)hexanoate

Bis(1,5-cyclooctadiene)diiridium (I) dichloride (31 mg, 0.046 mmol) and bis(diphenylphosphino)methane (35 mg, 0.090 mmol) were added to an oven-dried round-bottom flask. The flask was sealed and purged with N₂. The solids were dissolved in DCM (5 mL) and 4,4,5,5-tetramethyl-1,3,2-dioxaborolane (0.50 mL, 3.5 mmol) was added slowly to the solution. The reaction stirred at room temperature for 10 min. (2S,3S)-tert-butyl 2-(benzyloxycarbonylamino)-3-((1,3-dioxoisoindolin-2-yl)methyl)hex-5-enoate (Intermediate 45, 725 mg, 1.52 mmol) was added to the reaction as a solution in DCM (4 mL) and the reaction stirred at room temperature for 15 h. The reaction mixture was cooled to 0° C. and quenched with MeOH (2 mL) and water (10 mL). The layers were separated and the aqueous layer was extracted with DCM (2×10 mL). The combined organics were dried over MgSO₄, filtered and concentrated to dryness. The crude material was purified by silica gel chromatography (hexanes/EtOAc) to afford (2S,3S)-tert-butyl 2-(benzyloxycarbonylamino)-3-((1,3-dioxoisoindolin-2-yl)methyl)-6-(4,4,5,5-tetramethyl-1,3,2-dioxaborolan-2-yl)hexanoate (Intermediate 46, 612 mg, 67% yield) as a glassy, colorless residue. ¹H NMR (300 MHz, CDCl₃) δ 0.79 (2H, br t), 1.19 (12H, s), 1.21 (9H, s), 1.25-1.54 (3H, m), 1.59-1.77 (1H, m), 2.51-2.66 (1H, m), 3.49-3.67 (2H, m), 4.44 (1H, br d), 5.11 (2H, s), 5.96 (1H, br d), 7.26-7.41 (5H, m), 7.61-7.72 (2H, m), 7.75-7.85 (2H, m); m/z: (ES⁺) [M+NH₄]⁺=624.

### Example 18: (2S,3S)-2-amino-3-(aminomethyl)-6-boronohexanoic Acid Dihydrochloride

(2S,3S)-tert-Butyl 2-(benzyloxycarbonylamino)-3-((1,3-dioxoisoindolin-2-yl)methyl)-6-(4,4,5,5-tetramethyl-1,3,2-dioxaborolan-2-yl)hexanoate (Intermediate 46, 315 mg, 0.520 mmol) was dissolved in 6 M aq. HCl (5 mL) and the solution was heated to 100° C. for 16 h. The reaction was cooled to room temperature, diluted with water (10 mL) and washed with ether (3×10 mL). The aqueous layer was lyophilized and purified by reverse phase chromatography (RediSep Rf Gold® C18Aq, 0 to 100% MeCN in water) to afford (2S,3S)-2-amino-3-(aminomethyl)-6-boronohexanoic acid dihydrochloride (Example 18, 94 mg, 65% yield) as a yellow solid. ¹H NMR (300 MHz, D₂O) δ 0.73-0.91 (2H, m), 1.34-1.64 (4H, m), 2.29-2.45 (1H, m), 3.27 (2H, qd), 4.16 (1H, d); m/z: (ES⁺) [M−H₂O+H]⁺=187.

### Example 19: (2S,3S)-2-amino-3-(((S)-2-amino-3-methylbutanamido)methyl)-6-boronohexanoic Acid

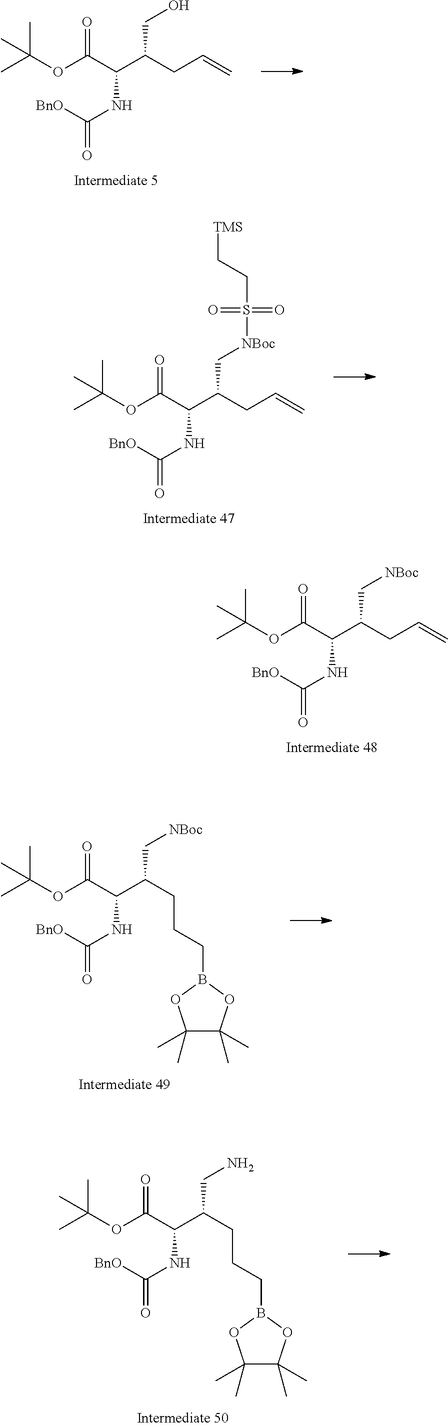

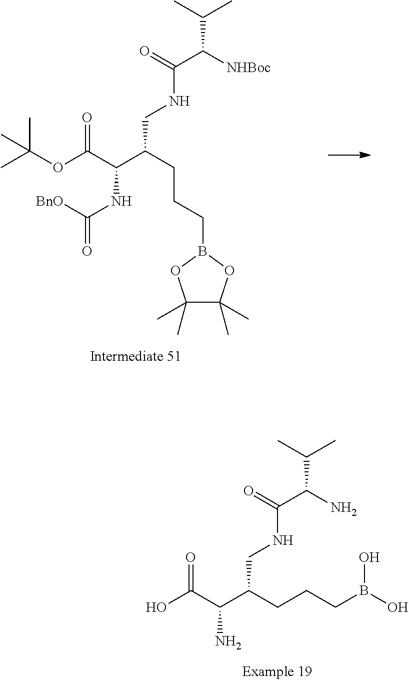 [SMILES: CC(C)[C@H](NC(=O)OC(C)(C)C)C(=O)NC[C@H](CCCB1OC(C)(C)C(C)(C)O1)[C@H](NC(=O)OCc1ccccc1)C(=O)OC(C)(C)C ; CC(C)[C@H](N)C(=O)NC[C@H](CCCB(O)O)[C@H](N)C(=O)O]

### Intermediate 47: (2S,3S)-tert-butyl 2-(benzyloxycarbonylamino)-3-((N-(tert-butoxycarbonyl)-2-(trimethylsilyl)ethylsulfonamido)methyl)hex-5-enoate

(2S,3R)-tert-Butyl 2-(benzyloxycarbonylamino)-3-(hydroxymethyl)hex-5-enoate (Intermediate 5, 747 mg, 2.14 mmol) and tert-butyl ((2-(trimethylsilyl)ethyl)sulfonyl)carbamate (602 mg, 2.14 mmol) were dissolved in THF (10 mL) and cooled to 0° C. Triphenylphosphine (842 mg, 3.21 mmol) and DIAD (0.85 mL, 4.4 mmol) were added and the reaction stirred for 16 h while slowly warming to room temperature. The reaction was quenched with saturated aqueous sodium bicarbonate (20 mL) and the layers were separated. The aqueous layer was extracted with EtOAc (2×15 mL). The combined organic layers were dried over MgSO₄, filtered, and concentrated to dryness. The crude material was purified by silica gel chromatography (hexanes/EtOAc) to afford (2S,3S)-tert-butyl 2-(benzyloxycarbonylamino)-3-((N-(tert-butoxycarbonyl)-2-(trimethylsilyl)ethylsulfonamido)methyl)hex-5-enoate (Intermediate 47, 860 mg, 66% yield). ¹H NMR (500 MHz, CDCl₃) 0.04 (9H, s), 0.91-1.01 (2H, m), 1.46 (9H, s), 1.50 (9H, s), 2.04-2.13 (2H, m), 2.41-2.65 (1H, m), 3.33-3.47 (2H, m), 3.58-3.81 (2H, m), 4.40 (1H, br d), 4.96-5.19 (4H, m), 5.66 (1H, br d), 5.72-5.96 (1H, m), 7.25-7.41 (5H, m); m/z: (ES⁺) [M+NH₄]⁺=630.

### Intermediate 48: (2S,3S)-tert-butyl 2-(benzyloxycarbonylamino)-3-((tert-butoxycarbonylamino)methyl)hex-5-enoate

A solution of TBAF (1 M in THF, 6.0 mL, 6.0 mmol) was added to a solution of (2S,3S)-tert-butyl 2-(benzyloxycarbonylamino)-3-((N-(tert-butoxycarbonyl)-2-(trimethylsilyl)ethylsulfonamido)methyl)hex-5-enoate (Intermediate 47, 1.23 g, 2.01 mmol) in THF (6 mL) and the reaction stirred at room temperature for 30 min. The reaction was diluted with Et₂O (30 mL) and washed sequentially with water (3×15 mL). The organic layer was dried over MgSO₄, filtered and concentrated to dryness. The crude material was purified by silica gel chromatography (hexanes/EtOAc) to afford (2S,3S)-tert-butyl 2-(benzyloxycarbonylamino)-3-((tert-butoxycarbonylamino)methyl)hex-5-enoate (Intermediate 48, 834 mg, 93% yield) as a pale yellow oil. ¹H NMR (500 MHz, CDCl₃) δ 1.41 (9H, s), 1.45 (9H, s), 1.90-1.99 (1H, m), 2.06-2.16 (1H, m), 2.17-2.30 (1H, m), 2.92-3.14 (1H, m), 3.14-3.27 (1H, m), 4.36 (1H, br dd), 4.46-4.69 (1H, m), 5.03-5.15 (4H, m), 5.70-5.92 (2H, m), 7.26-7.39 (5H, m); m/z: (ES⁺) [M+H]⁺=449.

### Intermediate 49: (2S,3S)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate

Bis(1,5-cyclooctadiene)diiridium (I) dichloride (48 mg, 0.071 mmol) and bis(diphenylphosphino)methane (55 mg, 0.14 mmol) were added to an oven-dried round-bottom flask. The flask was sealed and purged with N₂. The solids were dissolved in DCM (7 mL) and 4,4,5,5-tetramethyl-1,3,2-dioxaborolane (0.76 mL, 5.2 mmol) was added slowly to the solution. The reaction stirred at room temperature for 10 min. (2S,3S)-tert-Butyl 2-(benzyloxycarbonylamino)-3-((tert-butoxycarbonylamino)methyl)hex-5-enoate (Intermediate 48, 1.071 g, 2.39 mmol) was added to the reaction as a solution in DCM (6 mL) and the reaction stirred at room temperature for 15 h. The reaction mixture was cooled to 0° C. and quenched with MeOH (2 mL) and water (15 mL). The layers were separated and the aqueous layer was extracted with DCM (2×10 mL). The combined organics were dried over MgSO₄, filtered and concentrated to dryness. The crude material was purified by silica gel chromatography (hexanes/EtOAc) to afford (2S,3S)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate (Intermediate 49, 1.01 g, 73% yield) as a pale yellow gum. ¹H NMR (500 MHz, CDCl₃) δ 0.75 (2H, br t), 1.20 (12H, s), 1.30-1.38 (2H, m), 1.40 (9H, s), 1.43 (9H, s), 1.46-1.51 (2H, m), 2.09 (1H, br s), 2.98-3.12 (1H, m), 3.12-3.25 (1H, m), 4.30 (1H, br dd), 4.55-4.79 (1H, m), 5.01-5.18 (2H, m), 5.77 (1H, br d), 7.25-7.38 (5H, m); m/z: (ES⁺) [M+H]⁺=577.

### Intermediate 50: (2S,3S)-tert-butyl 3-(aminomethyl)-2-(benzyloxycarbonylamino)-6-(4,4,5,5-tetramethyl-1,3,2-dioxaborolan-2-yl)hexanoate

A solution of HCl (4 M in dioxane, 3.35 mL, 13.4 mmol) was added to a solution of (2S,3S)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate (Intermediate 49, 773 mg, 1.34 mmol) in dioxane (3.5 mL) at 0° C. The reaction stirred for 2 h while slowly warming to room temperature. The solution was concentrated to dryness to afford (2S,3S)-tert-butyl 3-(aminomethyl)-2-(benzyloxycarbonylamino)-6-(4,4,5,5-tetramethyl-1,3,2-dioxaborolan-2-yl)hexanoate (Intermediate 50, 639 mg, 100% yield) as an off-white solid which was used without further purification. m/z: (ES⁺) [M+H]⁺=476.

### Intermediate 51: (2S,3S)-tert-butyl 2-(benzyloxycarbonylamino)-3-(((S)-2-(tert-butoxycarbonylamino)-3-methylbutanamido)methyl)-6-(4,4,5,5-tetramethyl-1,3,2-dioxaborolan-2-yl)hexanoate

HATU (385 mg, 1.01 mmol) was added to a solution of Boc-Val-OH (220 mg, 1.01 mmol) in DMF (4 mL) and the reaction stirred at room temperature for 10 min. (2S,3S)-tert-Butyl 3-(aminomethyl)-2-(benzyloxycarbonylamino)-6-(4,4,5,5-tetramethyl-1,3,2-dioxaborolan-2-yl)hexanoate (Intermediate 50, 438 mg, 0.919 mmol) was then added to the reaction as a solution in DMF (6 mL). N,N-Diisopropylethylamine (0.80 mL, 4.6 mmol) was added and the reaction stirred at room temperature for 3 h. The reaction mixture was diluted with saturated aqueous NH₄Cl and DCM and the layers were separated. The aqueous layer was extracted with DCM (3×20 mL). The combined organics were dried over Na₂SO₄, filtered and concentrated to dryness. The crude material was purified by silica gel chromatography (hexanes/EtOAc) to afford (2S,3S)-tert-butyl 2-(benzyloxycarbonylamino)-3-(((S)-2-(tert-butoxycarbonylamino)-3-methylbutanamido)methyl)-6-(4,4,5,5-tetramethyl-1,3,2-dioxaborolan-2-yl)hexanoate (Intermediate 51, 450 mg, 72% yield) as a white foam. ¹H NMR (500 MHz, CDCl₃) δ 0.72 (2H, br t), 0.80-0.97 (6H, m), 1.13-1.35 (13H, m), 1.37-1.63 (21H, m), 1.95 (1H, br s), 2.07-2.17 (1H, m), 3.07-3.31 (1H, m), 3.34-3.54 (1H, m), 3.80-4.00 (1H, m), 4.14-4.28 (1H, m), 5.00-5.23 (3H, m), 5.50-5.78 (1H, m), 6.28-6.70 (1H, m), 7.25-7.45 (5H, m); m/z: (ES⁺) [M+H]⁺=676.

### Example 19: (2S,3S)-2-amino-3-(((S)-2-amino-3-methylbutanamido)methyl)-6-boronohexanoic Acid

Pd/C (10 wt %, 71 mg, 0.070 mmol) was added to a solution of (2S,3S)-tert-butyl 2-(benzyloxycarbonylamino)-3-(((S)-2-(tert-butoxycarbonylamino)-3-methylbutanamido)methyl)-6-(4,4,5,5-tetramethyl-1,3,2-dioxaborolan-2-yl)hexanoate (Intermediate 51, 450 mg, 0.67 mmol) in EtOAc (10 mL). The flask was equipped with a balloon of H₂ and the suspension stirred overnight at room temperature. The reaction mixture was filtered through diatomaceous earth and rinsed with EtOAc and methanol. The filtrate was concentrated to dryness and the resulting residue was dissolved in HCl (4 M in dioxane, 10.0 mL, 40.0 mmol). The reaction was heated to 50° C. and stirred for 1.5 h. The reaction was cooled to room temperature and concentrated. The resulting residue was dissolved in 1 M aq. HCl (15 ml) and Et₂O (15 mL). Phenylboronic acid (155 mg, 1.27 mmol) was added and the reaction stirred at room temperature for 4 h. The reaction mixture was diluted with water and washed with Et₂O. The aqueous layer was lyophilized and purified by ion exchange chromatography (PoraPak Rxn CX 20 cc column). The desired product was eluted from the column using 2.5 M ammonia/methanol to afford (2S,3S)-2-amino-3-(((S)-2-amino-3-methylbutanamido)methyl)-6-boronohexanoic acid (Example 19, 147 mg, 76% yield) as a white solid. ¹H NMR (500 MHz, D₂O) δ 0.70-0.85 (2H, m), 0.91-1.01 (6H, m), 1.29-1.56 (4H, m), 1.94-2.05 (1H, m), 2.17-2.31 (1H, m), 3.26-3.44 (3H, m), 3.70 (1H, d); m/z: (ES⁺) [M+H]⁺=304.

### Example 20: (2S,3S)-2-amino-3-(((S)-2-aminopropanamido)methyl)-6-boronohexanoic Acid

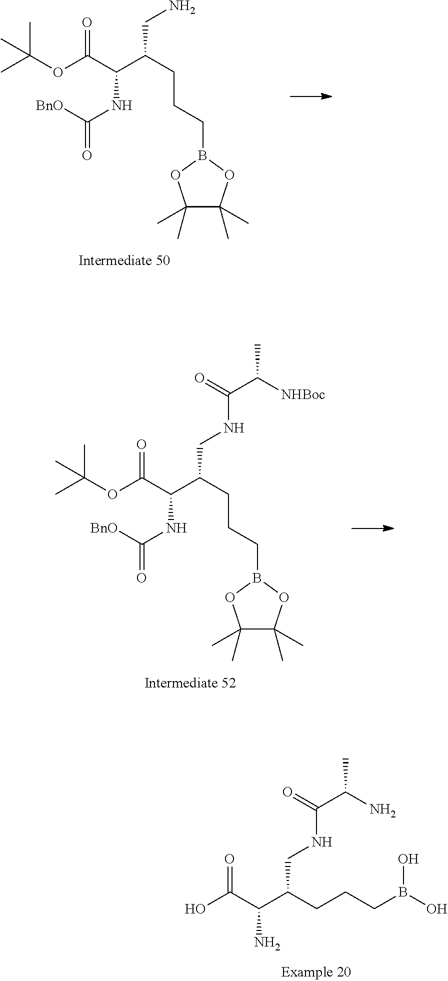 [SMILES: CC(C)(C)OC(=O)[C@@H](NC(=O)OCc1ccccc1)[C@H](CN)CCCB1OC(C)(C)C(C)(C)O1 ; C[C@H](NC(=O)OC(C)(C)C)C(=O)NC[C@H](CCCB1OC(C)(C)C(C)(C)O1)[C@H](NC(=O)OCc1ccccc1)C(=O)OC(C)(C)C ; C[C@H](N)C(=O)NC[C@H](CCCB(O)O)[C@H](N)C(=O)O]

### Intermediate 50 Intermediate 52 Example 20 Intermediate 52: (2S,3S)-tert-butyl 2-(benzyloxycarbonylamino)-3-(((S)-2-(tert-butoxycarbonylamino)propanamido)methyl)-6-(4,4,5,5-tetramethyl-1,3,2-dioxaborolan-2-yl)hexanoate

HATU (561 mg, 1.48 mmol) was added to a solution of Boc-Ala-OH (279 mg, 1.48 mmol) in DMF (4 mL) and the reaction stirred at room temperature for 10 min. (2S,3S)-tert-Butyl 3-(aminomethyl)-2-(benzyloxycarbonylamino)-6-(4,4,5,5-tetramethyl-1,3,2-dioxaborolan-2-yl)hexanoate (Intermediate 50, 639 mg, 1.34 mmol) was then added to the reaction as a solution in DMF (6 mL). N,N-Diisopropylethylamine (1.17 mL, 6.71 mmol) was added and the reaction stirred at room temperature for 3 h. The reaction mixture was diluted with saturated aqueous NH₄Cl and DCM and the layers were separated. The aqueous layer was extracted with DCM (3×30 mL). The combined organics were dried over Na₂SO₄, filtered and concentrated to dryness. The crude material was purified by silica gel chromatography (hexanes/EtOAc) to afford (2S,3S)-tert-butyl 2-(benzyloxycarbonylamino)-3-(((S)-2-(tert-butoxycarbonylamino)propanamido)methyl)-6-(4,4,5,5-tetramethyl-1,3,2-dioxaborolan-2-yl)hexanoate (Intermediate 52, 662 mg, 76% yield) as a white solid. ¹H NMR (500 MHz, CDCl₃) δ 0.65-0.78 (2H, m), 1.19 (12H, s), 1.32 (3H, br d), 1.37-1.55 (22H, m), 1.95-2.07 (1H, m), 3.10-3.27 (1H, m), 3.30-3.50 (1H, m), 3.87-4.45 (2H, m), 5.08 (2H, br s), 5.13-5.23 (1H, m), 5.76 (1H, br s), 6.52-6.80 (1H, m), 7.25-7.44 (5H, m); m/z: (ES⁺) [M+H]⁺=648.

### Example 20: (2S,3S)-2-amino-3-(((S)-2-aminopropanamido)methyl)-6-boronohexanoic Acid

Pd/C (10 wt %, 110 mg, 0.10 mmol) was added to a solution of (2S,3S)-tert-butyl 2-(benzyloxycarbonylamino)-3-(((S)-2-(tert-butoxycarbonylamino)propanamido)methyl)-6-(4,4,5,5-tetramethyl-1,3,2-dioxaborolan-2-yl)hexanoate (Intermediate 52, 662 mg, 1.02 mmol) in Et₂O (10 mL). The flask was equipped with a balloon of H₂ and the suspension stirred overnight at room temperature. The reaction mixture was filtered through diatomaceous earth and rinsed with EtOAc and methanol. The filtrate was concentrated to dryness and the resulting residue was dissolved in HCl (4 M in dioxane, 10.0 mL, 40.0 mmol) and the reaction stirred at room temperature for 3.5 h. The reaction mixture was concentrated and the resulting solid was triturated with Et₂O. The solid was dissolved in 1 M aq. HCl (15 ml) and Et₂O (15 mL). Phenylboronic acid (245 mg, 2.01 mmol) was added and the reaction stirred overnight at room temperature. The reaction mixture was diluted with water and washed with Et₂O. The aqueous layer was lyophilized and purified by ion exchange chromatography (Silicycle SPE-R51230B-20X column). The desired product was eluted from the column using 2.5 M ammonia/methanol to afford (2S,3S)-2-amino-3-(((S)-2-aminopropanamido)methyl)-6-boronohexanoic acid (Example 20, 250 mg, 91% yield) as a white solid. ¹H NMR (500 MHz, D₂O) δ 0.68-0.79 (2H, m), 1.26 (3H, br d), 1.29-1.36 (2H, m), 1.37-1.49 (2H, m), 2.10-2.27 (1H, m), 3.16-3.37 (2H, m), 3.50-3.64 (2H, m); m/z: (ES⁺) [M+H]⁺=276.

### Example 21: (2S,3S)-2-amino-3-(((S)-2-aminobutanamido)methyl)-6-boronohexanoic Acid

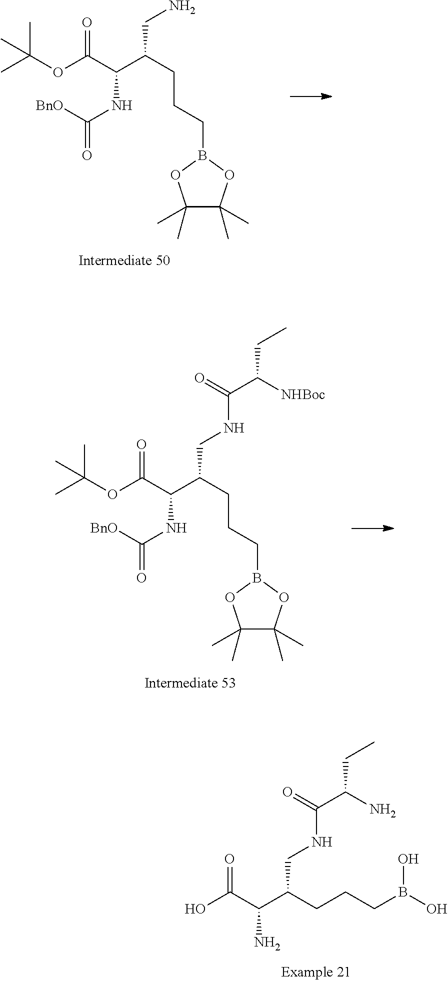 [SMILES: CC(C)(C)OC(=O)[C@@H](NC(=O)OCc1ccccc1)[C@H](CN)CCCB1OC(C)(C)C(C)(C)O1 ; CC[C@H](NC(=O)OC(C)(C)C)C(=O)NC[C@H](CCCB1OC(C)(C)C(C)(C)O1)[C@H](NC(=O)OCc1ccccc1)C(=O)OC(C)(C)C ; CC[C@H](N)C(=O)NC[C@H](CCCB(O)O)[C@H](N)C(=O)O]

### Intermediate 53: (2S,3S)-tert-butyl 2-(benzyloxycarbonylamino)-3-(((S)-2-(tert-butoxycarbonylamino)butanamido)methyl)-6-(4,4,5,5-tetramethyl-1,3,2-dioxaborolan-2-yl)hexanoate

N,N-Diisopropylethylamine (0.49 mL, 2.8 mmol) was added to a suspension of HATU (220 mg, 0.58 mmol) and Boc-Abu-OH (230 mg, 1.13 mmol) in DCM (3 mL) and the reaction stirred at room temperature for 10 min. DMF (1 mL) was added to the suspension and the reaction stirred at room temperature for an additional 5 min. A solution of (2S,3S)-tert-butyl 3-(aminomethyl)-2-(benzyloxycarbonylamino)-6-(4,4,5,5-tetramethyl-1,3,2-dioxaborolan-2-yl)hexanoate (Intermediate 50, 238 mg, 0.499 mmol) in DCM (2 mL) was added and the reaction stirred overnight at room temperature. The reaction mixture was diluted with water and DCM and the layers were separated. The aqueous layer was extracted with DCM (2×20 mL). The combined organics were washed with saturated sodium chloride, dried over Na₂SO₄, filtered and concentrated to dryness. The crude material was purified by silica gel chromatography (hexanes/EtOAc) to afford (2S,3S)-tert-butyl 2-(benzyloxycarbonylamino)-3-(((S)-2-(tert-butoxycarbonylamino)butanamido)methyl)-6-(4,4,5,5-tetramethyl-1,3,2-dioxaborolan-2-yl)hexanoate (Intermediate 53, 110 mg, 29% yield). ¹H NMR (500 MHz, CDCl₃) δ 0.73 (2H, br s), 0.91 (3H, br s), 1.21 (13H, br s), 1.30-1.37 (1H, m), 1.39-1.43 (9H, m), 1.45 (9H, br s), 1.49-1.65 (2H, m), 1.86 (1H, br s), 2.00 (1H, br s), 2.14-2.69 (1H, m), 3.20 (1H, br s), 3.31-3.61 (1H, m), 3.99 (1H, br s), 4.13-4.34 (1H, m), 5.09 (3H, br s), 5.66-5.88 (1H, m), 6.46-6.72 (1H, m), 7.28-7.39 (5H, m).

### Example 21: (2S,3S)-2-amino-3-(((S)-2-aminobutanamido)methyl)-6-boronohexanoic Acid

Pd/C (10 wt %, 43 mg, 0.040 mmol) was added to a solution of (2S,3S)-tert-butyl 2-(benzyloxycarbonylamino)-3-(((S)-2-(tert-butoxycarbonylamino)butanamido)methyl)-6-(4,4,5,5-tetramethyl-1,3,2-dioxaborolan-2-yl)hexanoate (Intermediate 53, 107 mg, 0.162 mmol) in ethyl acetate (5 mL). The flask was equipped with a balloon of H₂ and the suspension stirred overnight at room temperature. The reaction mixture was filtered through diatomaceous earth and rinsed with EtOAc and methanol. The filtrate was concentrated and the resulting residue was dissolved in DCM (1 mL) and trifluoroacetic acid (3 mL) and the reaction stirred at room temperature overnight. The reaction was concentrated and the residue was dissolved in 1 M aq. HCl (2 mL) and Et₂O (2 mL). Phenylboronic acid (38 mg, 0.31 mmol) was added and the reaction stirred at room temperature for 3 h. The reaction mixture was diluted with water and washed with Et₂O. The aqueous layer was lyophilized and purified by ion exchange chromatography (PoraPak Rxn CX 20 cc column). The desired product was eluted from the column using a 5% solution of ammonia in methanol to afford (2S,3S)-2-amino-3-(((S)-2-aminobutanamido)methyl)-6-boronohexanoic acid (Example 21, 40 mg, 89% yield) as a white solid. ¹H NMR (500 MHz, D₂O) δ 0.74 (2H, td), 0.85 (3H, br t), 1.27-1.36 (2H, m), 1.37-1.47 (2H, m), 1.56-1.71 (2H, m), 2.11-2.20 (1H, m), 3.20-3.35 (2H, m), 3.39 (1H, t), 3.60 (1H, d); m/z: (ES⁺) [M+H]⁺=290.

### Example 22: (2S,3S)-2-amino-3-(((2S,3S)-2-amino-3-methylpentanamido)methyl)-6-boronohexanoic Acid

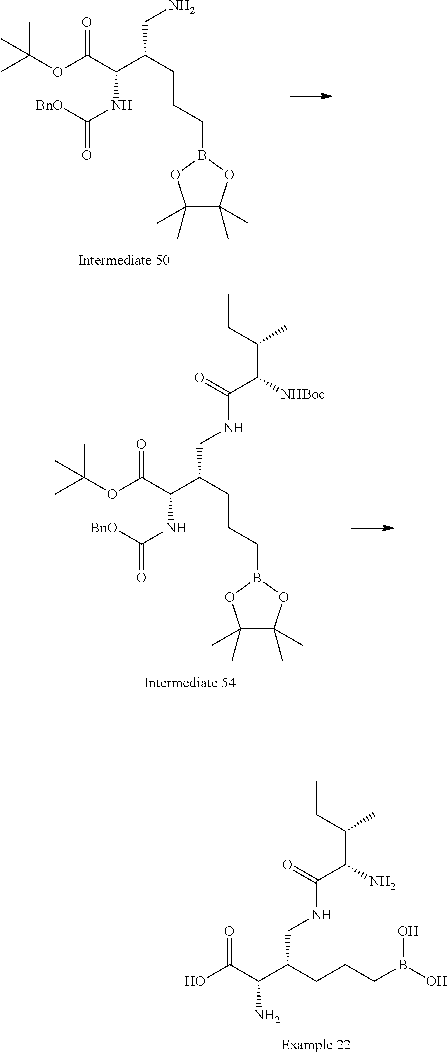 [SMILES: CC(C)(C)OC(=O)[C@@H](NC(=O)OCc1ccccc1)[C@H](CN)CCCB1OC(C)(C)C(C)(C)O1 ; CC[C@H](C)[C@H](NC(=O)OC(C)(C)C)C(=O)NC[C@H](CCCB1OC(C)(C)C(C)(C)O1)[C@H](NC(=O)OCc1ccccc1)C(=O)OC(C)(C)C ; CC[C@H](C)[C@H](N)C(=O)NC[C@H](CCCB(O)O)[C@H](N)C(=O)O]

### Intermediate 54: (2S,3S)-tert-butyl 2-(benzyloxycarbonylamino)-3-(((2S,3S)-2-(tert-butoxycarbonylamino)-3-methylpentanamido)methyl)-6-(4,4,5,5-tetramethyl-1,3,2-dioxaborolan-2-yl)hexanoate

N,N-Diisopropylethylamine (0.84 mL, 4.8 mmol) was added to a suspension of HATU (365 mg, 0.960 mmol) and Boc-Ile-OH (462 mg, 2.00 mmol) in DCM (3 mL) and DMF (3 mL) and the reaction stirred at room temperature for 10 min. A solution of (2S,3S)-tert-butyl 3-(aminomethyl)-2-(benzyloxycarbonylamino)-6-(4,4,5,5-tetramethyl-1,3,2-dioxaborolan-2-yl)hexanoate (Intermediate 50, 336 mg, 0.705 mmol) in DCM (3 mL) was added and the reaction stirred overnight at room temperature. The reaction mixture was diluted with water and DCM and the layers were separated. The aqueous layer was extracted with DCM (2×20 mL). The combined organics were washed with saturated sodium bicarbonate, dried over Na₂SO₄, filtered and concentrated to dryness. The crude material was purified by silica gel chromatography (hexanes/EtOAc) to afford (2S,3S)-tert-butyl 2-(benzyloxycarbonylamino)-3-(((2S,3S)-2-(tert-butoxycarbonylamino)-3-methylpentanamido)methyl)-6-(4,4,5,5-tetramethyl-1,3,2-dioxaborolan-2-yl)hexanoate (Intermediate 54, 260 mg, 47% yield). ¹H NMR (500 MHz, CDCl₃) δ 0.73 (2H, br t), 0.83-0.97 (6H, m), 1.08 (1H, br s), 1.21 (13H, br s), 1.29-1.63 (22H, m), 1.82-1.92 (1H, m), 1.92-2.04 (1H, m), 3.18 (1H, br s), 3.43 (1H, br d), 3.96 (1H, br s), 4.21 (1H, br d), 5.09 (3H, br s), 5.58-5.80 (1H, m), 6.40-6.69 (1H, m), 7.29-7.40 (5H, m).

### Example 22: (2S,3S)-2-amino-3-(((2S,3S)-2-amino-3-methylpentanamido)methyl)-6-boronohexanoic Acid

Pd/C (10 wt %, 99 mg, 0.093 mmol) was added to a solution of (2S,3S)-tert-butyl 2-(benzyloxycarbonylamino)-3-(((2S,3S)-2-(tert-butoxycarbonylamino)-3-methylpentanamido)methyl)-6-(4,4,5,5-tetramethyl-1,3,2-dioxaborolan-2-yl)hexanoate (Intermediate 54, 256 mg, 0.371 mmol) in ethyl acetate (8 mL). The flask was equipped with a balloon of H₂ and the suspension stirred overnight at room temperature. The reaction mixture was filtered through diatomaceous earth and rinsed with EtOAc and methanol. The filtrate was concentrated and the resulting residue was dissolved in DCM (2 mL) and trifluoroacetic acid (6 mL) and the reaction stirred at room temperature for 3 h. The reaction was concentrated and the residue was dissolved in 1 M aq. HCl (2 mL) and Et₂O (5 mL). Phenylboronic acid (82 mg, 0.67 mmol) was added and the reaction stirred at room temperature overnight. The reaction mixture was diluted with water and washed with Et₂O. The aqueous layer was lyophilized and purified by ion exchange chromatography (PoraPak Rxn CX 20 cc column). The desired product was eluted from the column using a 5% solution of ammonia in methanol to afford (2S,3S)-2-amino-3-(((2S,3S)-2-amino-3-methylpentanamido)methyl)-6-boronohexanoic acid (Example 22, 93 mg, 87% yield) as a white solid. ¹H NMR (500 MHz, D₂O) δ 0.68-0.78 (2H, m), 0.80-0.91 (6H, m), 1.07-1.18 (1H, m), 1.27-1.48 (5H, m), 1.69 (1H, br d), 2.14 (1H, br d), 3.21-3.36 (3H, m), 3.62 (1H, br d); m/z: (ES⁺) [M+H]⁺=318.

### Example 23: (2S,3S)-2-amino-3-(((S)-2-amino-3,3-dimethylbutanamido)methyl)-6-boronohexanoic Acid

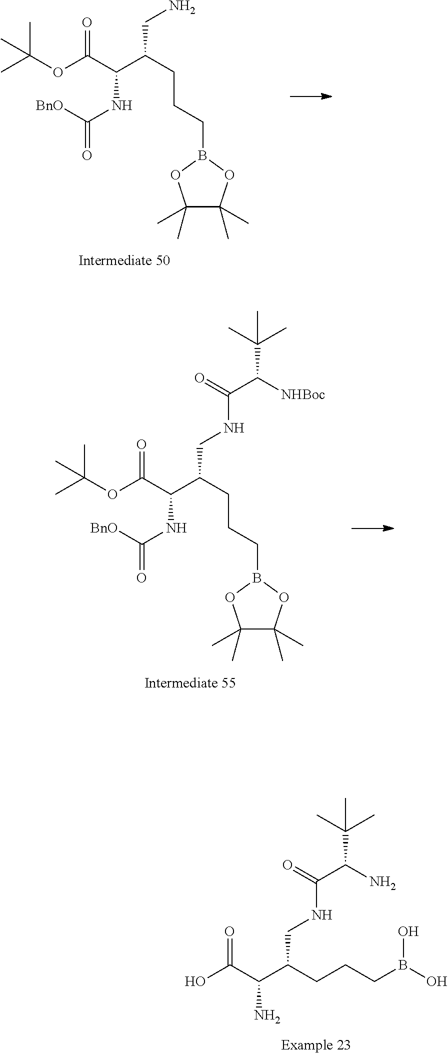 [SMILES: CC(C)(C)OC(=O)[C@@H](NC(=O)OCc1ccccc1)[C@H](CN)CCCB1OC(C)(C)C(C)(C)O1 ; CC(C)(C)OC(=O)N[C@H](C(=O)NC[C@H](CCCB1OC(C)(C)C(C)(C)O1)[C@H](NC(=O)OCc1ccccc1)C(=O)OC(C)(C)C)C(C)(C)C ; CC(C)(C)[C@H](N)C(=O)NC[C@H](CCCB(O)O)[C@H](N)C(=O)O]

### Intermediate 55: (2S,3S)-tert-butyl 2-(benzyloxycarbonylamino)-3-(((S)-2-(tert-butoxycarbonylamino)-3,3-dimethylbutanamido)methyl)-6-(4,4,5,5-tetramethyl-1,3,2-dioxaborolan-2-yl)hexanoate

N,N-Diisopropylethylamine (1.08 mL, 6.20 mmol) was added to a suspension of HATU (472 mg, 1.24 mmol) and Boc-Tle-OH (550 mg, 2.4 mmol) in DCM (3 mL) and DMF (3 mL) and the reaction stirred at room temperature for 10 min. A solution of (2S,3S)-tert-butyl 3-(aminomethyl)-2-(benzyloxycarbonylamino)-6-(4,4,5,5-tetramethyl-1,3,2-dioxaborolan-2-yl)hexanoate (Intermediate 50, 434 mg, 0.911 mmol) in DCM (4.5 mL) was added and the reaction stirred overnight at room temperature. The reaction mixture was diluted with water and DCM and the layers were separated. The aqueous layer was extracted with DCM (2×20 mL). The combined organics were washed with saturated sodium bicarbonate, dried over Na₂SO₄, filtered and concentrated to dryness. The crude material was purified by silica gel chromatography (hexanes/EtOAc) to afford (2S,3S)-tert-butyl 2-(benzyloxycarbonylamino)-3-(((S)-2-(tert-butoxycarbonylamino)-3,3-dimethylbutanamido)methyl)-6-(4,4,5,5-tetramethyl-1,3,2-dioxaborolan-2-yl)hexanoate (Intermediate 55, 560 mg, 79% yield) as a colorless oil. ¹H NMR (500 MHz, CDCl₃) δ 0.75 (2H, br t), 0.95-1.05 (9H, m), 1.18-1.25 (13H, m), 1.32-1.40 (1H, m), 1.42-1.45 (9H, m), 1.46-1.51 (9H, m), 1.52-1.62 (1H, m), 1.64-1.73 (1H, m), 1.88-2.04 (1H, m), 3.08-3.27 (1H, m), 3.39-3.56 (1H, m), 3.74-3.90 (1H, m), 4.18-4.30 (1H, m), 5.05-5.17 (2H, m), 5.31 (1H, s), 5.47-5.80 (1H, m), 6.14-6.44 (1H, m), 7.30-7.40 (5H, m); m/z: (ES⁺) [M+H]⁺=690.

### Example 23: (2S,3S)-2-amino-3-(((S)-2-amino-3,3-dimethylbutanamido)methyl)-6-boronohexanoic Acid

Pd/C (10 wt %, 138 mg, 0.130 mmol) was added to a solution of (2S,3S)-tert-butyl 2-(benzyloxycarbonylamino)-3-(((S)-2-(tert-butoxycarbonylamino)-3,3-dimethylbutanamido)methyl)-6-(4,4,5,5-tetramethyl-1,3,2-dioxaborolan-2-yl)hexanoate (Intermediate 55, 359 mg, 0.521 mmol) in ethyl acetate (12 mL). The flask was equipped with a balloon of H₂ and the suspension stirred overnight at room temperature. The reaction mixture was filtered through diatomaceous earth and rinsed with EtOAc and methanol. The filtrate was concentrated and the resulting residue was dissolved in DCM (3 mL) and trifluoroacetic acid (9 mL) and the reaction stirred at room temperature for 3 h. The reaction was concentrated and the residue was dissolved in 1 M aq. HCl (3 mL) and Et₂O (5 mL). Phenylboronic acid (127 mg, 1.04 mmol) was added and the reaction stirred at room temperature overnight. The reaction mixture was diluted with water and washed with Et₂O. The aqueous layer was lyophilized and purified by ion exchange chromatography (PoraPak Rxn CX 20 cc column). The desired product was eluted from the column using a 5% solution of ammonia in methanol. The obtained material was further purified by reverse phase chromatography (RediSep Rf Gold® C18Aq, 0 to 10% acetonitrile in water) to afford (2S,3S)-2-amino-3-(((S)-2-amino-3,3-dimethylbutanamido)methyl)-6-boronohexanoic acid (Example 23, 96 mg, 58% yield) as a white solid. ¹H NMR (500 MHz, D₂O w/TFA) δ 0.52-0.73 (2H, m), 0.90 (9H, s), 1.17-1.41 (4H, m), 2.11-2.23 (1H, m), 3.21-3.34 (2H, m), 3.49-3.55 (1H, m), 3.91-4.00 (1H, m); m/z: (ES⁺) [M+H]⁺=318.

### Example 24: (2S,3S)-2-amino-3-((2-aminoacetamido)methyl)-6-boronohexanoic Acid

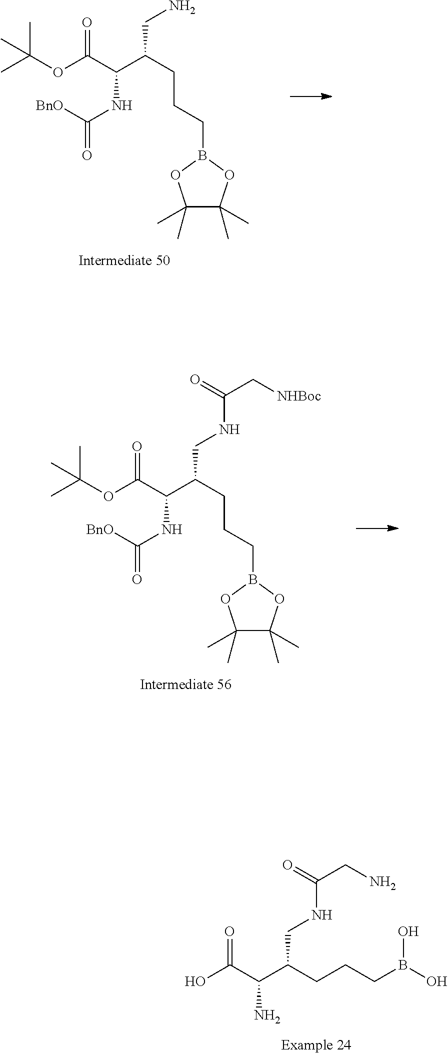 [SMILES: CC(C)(C)OC(=O)[C@@H](NC(=O)OCc1ccccc1)[C@H](CN)CCCB1OC(C)(C)C(C)(C)O1 ; CC(C)(C)OC(=O)NCC(=O)NC[C@H](CCCB1OC(C)(C)C(C)(C)O1)[C@H](NC(=O)OCc1ccccc1)C(=O)OC(C)(C)C ; NCC(=O)NC[C@H](CCCB(O)O)[C@H](N)C(=O)O]

### Intermediate 50 Intermediate 56 Example 24 Intermediate 56: (2S,3S)-tert-butyl 2-(benzyloxycarbonylamino)-3-((2-(tert-butoxycarbonylamino)acetamido)meth yl)-6-(4,4,5,5-tetramethyl-1,3,2-dioxaborolan-2-yl)hexanoate

N,N-Diisopropylethylamine (0.92 mL, 5.3 mmol) was added to a suspension of HATU (434 mg, 1.14 mmol) and Boc-Gly-OH (400 mg, 2.28 mmol) in DCM (3 mL) and DMF (3 mL) and the reaction stirred at room temperature for 10 min. A solution of (2S,3S)-tert-butyl 3-(aminomethyl)-2-(benzyloxycarbonylamino)-6-(4,4,5,5-tetramethyl-1,3,2-dioxaborolan-2-yl)hexanoate (Intermediate 50, 369 mg, 0.775 mmol) in DCM (3 mL) was added and the reaction stirred overnight at room temperature. The reaction mixture was diluted with water and DCM and the layers were separated. The aqueous layer was extracted with DCM (2×20 mL). The combined organics were washed with saturated sodium chloride, dried over Na₂SO₄, filtered and concentrated to dryness. The crude material was purified by silica gel chromatography (hexanes/EtOAc) to afford (2S,3S)-tert-butyl 2-(benzyloxycarbonylamino)-3-((2-(tert-butoxycarbonylamino)acetamido)methyl)-6-(4,4,5,5-tetramethyl-1,3,2-dioxaborolan-2-yl)hexanoate (Intermediate 56, 259 mg, 47% yield). ¹H NMR (500 MHz, CDCl₃) δ 0.72 (2H, br s), 1.19 (13H, br s), 1.28-1.36 (1H, m), 1.36-1.45 (18H, m), 1.45-1.56 (2H, m), 2.03-2.13 (1H, m), 3.12 (1H, br s), 3.41 (1H, br d), 3.61-3.89 (2H, m), 4.16-4.38 (1H, m), 5.07 (2H, br s), 5.30-5.43 (1H, m), 5.95 (1H, br s), 6.73 (1H, br s), 7.22-7.36 (5H, m).

### Example 24: (2S,3S)-2-amino-3-((2-aminoacetamido)methyl)-6-boronohexanoic Acid

Pd/C (10 wt %, 107 mg, 0.101 mmol) was added to a solution of (2S,3S)-tert-butyl 2-(benzyloxycarbonylamino)-3-((2-(tert-butoxycarbonylamino)acetamido)methyl)-6-(4,4,5,5-tetramethyl-1,3,2-dioxaborolan-2-yl)hexanoate (Intermediate 56, 256 mg, 0.404 mmol) in ethyl acetate (8 mL). The flask was equipped with a balloon of H₂ and the suspension stirred overnight at room temperature. The reaction mixture was filtered through diatomaceous earth and rinsed with EtOAc and methanol. The filtrate was concentrated and the resulting residue was dissolved in DCM (2 mL) and trifluoroacetic acid (6 mL) and the reaction stirred at room temperature for 3 h. The reaction was concentrated and the residue was dissolved in 1 M aq. HCl (3 mL) and Et₂O (5 mL). Phenylboronic acid (98 mg, 0.80 mmol) was added and the reaction stirred at room temperature overnight. The reaction mixture was diluted with water and washed with Et₂O. The aqueous layer was lyophilized and purified by ion exchange chromatography (PoraPak Rxn CX 20 cc column). The desired product was eluted from the column using a 5% solution of ammonia in methanol. The obtained material was further purified by reverse phase chromatography (RediSep Rf Gold® C18Aq, 0 to 5% acetonitrile in water) to afford (2S,3S)-2-amino-3-((2-aminoacetamido)methyl)-6-boronohexanoic acid (Example 24, 20 mg, 19% yield) as a white solid. ¹H NMR (500 MHz, D₂O w/TFA) δ 0.53-0.74 (2H, m), 1.16-1.45 (4H, m), 2.17-2.31 (1H, m), 3.16-3.36 (2H, m), 3.57-3.71 (2H, m), 3.96 (1H, d); m/z: (ES⁺) [M+H]⁺=262.

### Example 25: (2S,3S)-3-(aminomethyl)-2-[[(2S)-2-aminopropanoyl]amino]-6-borono-hexanoic acid

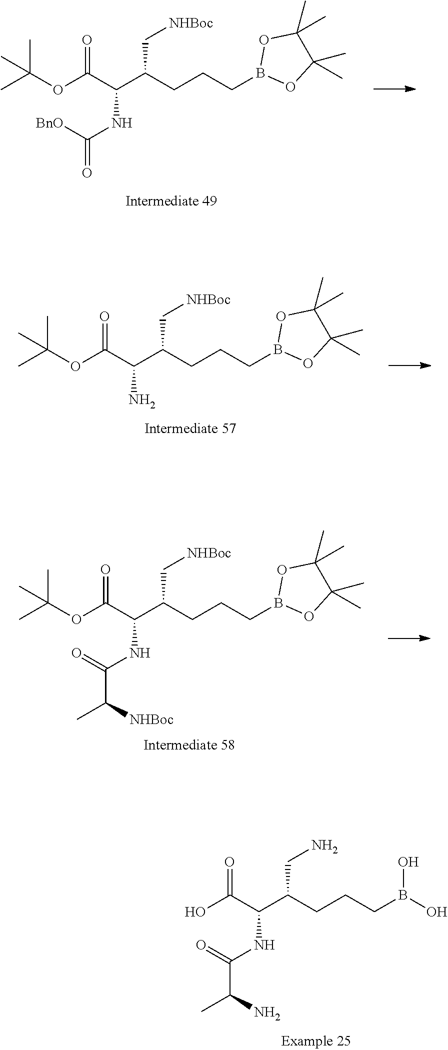

### Intermediate 57: tert-butyl (2S,3S)-2-amino-3-[(tert-butoxycarbonylamino)methyl]-6-(4,4,5,5-tetramethyl-1,3,2-dioxaborolan-2-yl)hexanoate

Pd/C (10 wt %, 243 mg, 0.228 mmol) was added to a solution of (2S,3S)-tert-butyl 2-(benzyloxycarbonylamino)-3-((tert-butoxycarbonylamino)methyl)-6-(4,4,5,5-tetramethyl-1,3,2-dioxaborolan-2-yl)hexanoate (Intermediate 49, 1.40 g, 2.43 mmol) in EtOAc (50 mL). The flask was equipped with a balloon of H₂ and the suspension stirred overnight at room temperature. The reaction mixture was filtered through diatomaceous earth and rinsed with EtOAc. The filtrate was concentrated to dryness to afford the crude material as a colorless oil. Crude material was subjected to chiral SFC [Chiral Pak IC column, 21×250 mm, 5 μm, Temperature=40° C., Mobile phase=15% isopropanol (with 0.2% NH₄OH):CO₂, flow rate=4 mL/min, Outlet pressure=100 bar] to afford tert-butyl (2S,3S)-2-amino-3-[(tert-butoxycarbonylamino)methyl]-6-(4,4,5,5-tetramethyl-1,3,2-dioxaborolan-2-yl)hexanoate (Intermediate 57, 855 mg, 80% yield, >98:2 dr) as a colorless oil. ¹H NMR (500 MHz, CDCl₃) δ 0.80 (2H, t), 1.19-1.33 (14H, m), 1.35-1.56 (20H, m), 1.61-1.85 (2H, m), 1.88-2.00 (1H, m), 3.10-3.31 (2H, m), 3.39 (1H, d), 5.25 (1H, br s); m/z: (ES⁺) [M+H]⁺=444.

### Intermediate 58: tert-butyl (2S,3S)-3-[(tert-butoxycarbonylamino)methyl]-2-[[(2S)-2-(tert-butoxycarbonylamino)propanoyl]amino]-6-(4,4,5,5-tetramethyl-1,3,2-dioxaborolan-2-yl)hexanoate

HATU (142 mg, 0.373 mmol) was added to a solution of Boc-Ala-OH (70 mg, 0.37 mmol) in DMF (6 mL) and the reaction stirred at room temperature for 10 min. tert-Butyl (2S,3S)-2-amino-3-[(tert-butoxycarbonylamino)methyl]-6-(4,4,5,5-tetramethyl-1,3,2-dioxaborolan-2-yl)hexanoate (Intermediate 57, 150 mg, 0.34 mmol) was then added to the reaction as a solution in DMF (2 mL). N,N-Diisopropylethylamine (0.12 mL, 0.68 mmol) was added and the reaction stirred at room temperature overnight. The reaction mixture was diluted with saturated aqueous NH₄Cl and DCM and the layers were separated. The aqueous layer was extracted with DCM (3×20 mL). The combined organics were dried over Na₂SO₄, filtered and concentrated to dryness. The crude material was purified by silica gel chromatography (hexanes/EtOAc) to afford tert-butyl (2S,3S)-3-[(tert-butoxycarbonylamino)methyl]-2-[[(2S)-2-(tert-butoxycarbonylamino)propanoyl]amino]-6-(4,4,5,5-tetramethyl-1,3,2-dioxaborolan-2-yl)hexanoate (Intermediate 58, 125 mg, 60% yield) as a white solid. ¹H NMR (500 MHz, CDCl₃) δ 0.71-0.82 (2H, m), 1.16-1.36 (14H, m), 1.37-1.69 (34H, m), 2.14-2.28 (1H, m), 3.01-3.10 (1H, m), 3.19-3.35 (1H, m), 4.21-4.36 (1H, m), 4.52-4.63 (1H, m), 4.66-4.80 (1H, m), 5.21-5.32 (1H, m), 7.13-7.25 (1H, m).

### Example 25: (2S,3S)-3-(aminomethyl)-2-[[(2S)-2-aminopropanoyl]amino]-6-borono-hexanoic Acid

tert-Butyl (2S,3S)-3-[(tert-butoxycarbonylamino)methyl]-2-[[(2S)-2-(tert-butoxycarbonylamino)propanoyl]amino]-6-(4,4,5,5-tetramethyl-1,3,2-dioxaborolan-2-yl)hexanoate (Intermediate 58, 125 mg, 0.204 mmol) was dissolved in HCl (4 M in dioxane, 7.0 mL, 28 mmol) and the reaction was heated to 50° C. and stirred for 2 h. The reaction mixture was cooled to room temperature and the solvent was removed under reduced pressure. The resulting white solid was dissolved in 1 M aq. HCl (10 mL) and Et₂O (10 mL). Phenylboronic acid (49 mg, 0.41 mmol) was added and the reaction stirred at room temperature for 1 h. The reaction mixture was diluted with water and washed with Et₂O. The aqueous layer was lyophilized and purified by ion exchange chromatography (PoraPak Rxn CX 20 cc column). The desired product was eluted from the column using 2.5 M ammonia/methanol. The obtained material was further purified by reverse phase chromatography (RediSep Rf Gold® C18Aq, 0 to 20% acetonitrile in water) to afford (2S,3S)-3-(aminomethyl)-2-[[(2S)-2-aminopropanoyl]amino]-6-borono-hexanoic acid (Example 25, 38 mg, 67% yield) as a white solid. ¹H NMR (500 MHz, D₂O) δ 0.74 (2H, t), 1.31 (3H, d), 1.33-1.54 (4H, m), 2.14-2.25 (1H, m), 2.98-3.04 (1H, m), 3.06-3.15 (1H, m), 3.64 (1H, q), 4.36 (1H, d); m/z: (ES⁺) [M+H]⁺=276.

### Example 26: (2S,3S)-3-(aminomethyl)-2-[[(2S)-2-amino-3-methyl-butanoyl]amino]-6-borono-hexanoic Acid

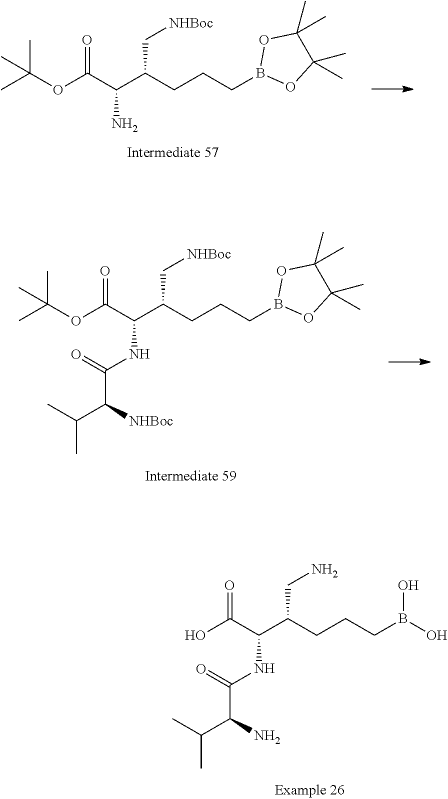

### Intermediate 59: tert-butyl (2S,3S)-3-[(tert-butoxycarbonylamino)methyl-2-[[(2S)-2-(tert-butoxycarbonylamino)-3-methyl-butanoyl]amino]-6-(4,4,5,5-tetramethyl-1,3,2-dioxaborolan-2-yl)hexanoate

HATU (326 mg, 0.857 mmol) was added to a solution of Boc-Val-OH (186 mg, 0.857 mmol) in DMF (10 mL) and the reaction stirred at room temperature for 10 min. tert-Butyl (2S,3S)-2-amino-3-[(tert-butoxycarbonylamino)methyl]-6-(4,4,5,5-tetramethyl-1,3,2-dioxaborolan-2-yl)hexanoate (Intermediate 57, 345 mg, 0.780 mmol) was then added to the reaction as a solution in DMF (5 mL). N,N-Diisopropylethylamine (0.27 mL, 1.6 mmol) was added and the reaction stirred at room temperature overnight. The reaction mixture was diluted with saturated aqueous NH₄Cl and DCM and the layers were separated. The aqueous layer was extracted with DCM (3×40 mL). The combined organics were dried over Na₂SO₄, filtered and concentrated to dryness. The crude material was purified by silica gel chromatography (hexanes/EtOAc) to afford tert-butyl (2S,3S)-3-[(tert-butoxycarbonylamino)methyl]-2-[[(2S)-2-(tert-butoxycarbonylamino)-3-methyl-butanoyl]amino]-6-(4,4,5,5-tetramethyl-1,3,2-dioxaborolan-2-yl)hexanoate (Intermediate 59, 422 mg, 84% yield) as a white solid. ¹H NMR (500 MHz, CDCl₃) δ 0.77 (2H, m), 0.90-1.04 (6H, m), 1.13-1.36 (14H, m), 1.37-1.72 (31H, m), 2.12-2.31 (2H, m), 2.94-3.08 (1H, m), 3.18-3.37 (1H, m), 4.00-4.11 (1H, m), 4.61 (1H, br d), 4.70 (1H, br d), 5.22 (1H, m), 7.08-7.20 (1H, m).

### Example 26: (2S,3S)-3-(aminomethyl)-2-[[(2S)-2-amino-3-methyl-butanoyl]amino]-6-borono-hexanoic Acid

tert-Butyl (2S,3S)-3-[(tert-butoxycarbonylamino)methyl]-2-[[(2S)-2-(tert-butoxycarbonylamino)-3-methyl-butanoyl]amino]-6-(4,4,5,5-tetramethyl-1,3,2-dioxaborolan-2-yl)hexanoate (Intermediate 59, 422, 0.658 mmol) was dissolved in HCl (4 M in dioxane, 10.0 mL, 48.0 mmol) and the reaction was heated to 50° C. and stirred for 2 h. The reaction mixture was cooled to room temperature and the solvent was removed under reduced pressure. The resulting white solid was dissolved in 1 M aq. HCl (15 mL) and Et₂O (15 mL). Phenylboronic acid (160 mg, 1.32 mmol) was added and the reaction stirred at room temperature for 1 h. The reaction mixture was diluted with water and washed with Et₂O. The aqueous layer was lyophilized and purified by ion exchange chromatography (Silicycle SPE-R51230B-20X column). The desired product was eluted from the column using 2.5 M ammonia/methanol. The obtained material was further purified by reverse phase chromatography (RediSep Rf Gold® C18Aq, 0 to 20% acetonitrile in water) to afford (2S,3S)-3-(aminomethyl)-2-[[(2S)-2-amino-3-methyl-butanoyl]amino]-6-borono-hexanoic acid (Example 26, 162 mg, 81% yield) as a white solid. ¹H NMR (500 MHz, D₂O) δ 0.76 (2H, t), 0.91 (3H, d), 0.95 (3H, d), 1.28-1.52 (4H, m), 1.99 (1H, dq), 2.18 (1H, dq), 2.98-3.07 (1H, m), 3.08-3.17 (1H, m), 3.31 (1H, d), 4.37 (1H, d); m/z: (ES⁺) [M+H]⁺=304.

### Example 27: Biological Activity of Examples 1-26

The inhibitory effects of Examples 1-26 on the activity of Human Arginase 1 and Arginase 2 activity were quantified by measuring the formation of the thiol group from thioarginine using recombinant Arginase 1 or Arginase 2 produced from E. coli. The thiol group was detected with Ellman's reagent, 5,5′-dithiobis(2-nitrobenzoic acid) (DTNB). DTNB reacts with the thiol to give the mixed disulfide and 2-nitro-5-thiobenzoic acid (TNB) which is quantified by the absorbance of the anion (TNB²⁻) at 412 nm.

The assays were run in clear 384 well plates (Greiner cat no: 781101). Various concentrations of Examples 1-26 in 300 nL DMSO were dispensed to assay plates using an Echo acoustic dispenser immediately followed by plate sealing and centrifugation. Two pre-mixes were prepared from reagents thawed immediately before addition to assay plates. Pre-mix one comprised human Arginase 1 or human Arginase 2, at a final concentration of 5 nM and 0.5 mM DTNB in assay buffer, 45 mM HEPES pH7.5, brij 35, 0.045% (w/v) and 100 μM MnCl₂. Pre-mix two comprised freshly thawed 0.5 mM thioarginine in assay buffer. Fifteen microlitres of pre-mix one was dispensed to assay plates containing Examples 1-9, centrifuged and incubated for 30 minutes at room temperature prior to adding fifteen microlitres of pre-mix two.

Assay plates were centrifuged prior to reading absorbance at 412 nm in a Pherastar multi-mode plate reader to collect data at time point 0 (T0). The plates were incubated at room temperature for 60 min prior to reading again to collect data at time point 1 (T1). Data is derived by subtracting the A412 signal measured at T0 (time point 0) from that measured at T1 (time point 1). The data was transformed to % effect using the equation:

Compound % effect=100*[(X−min)/(max−min)],

where X represents the normalized value for the compound based on the Min (vehicle) and Max (reference compound) inhibition control.

The concentration of Examples 1-26 that inhibited the activity by 50% (i.e.the IC₅₀) was calculated by plotting the % effect versus test compound concentration and fitting the data using the Genedata Screener Smart fit algorithm. The results of these assays are found in Table 2:

TABLE 2

| | Human Arginase 1 Enzyme | Human Arginase 2 Enzyme |
|---|---|---|
| Example | IC₅₀ (μM) | IC₅₀ (μM) |
| 1 | 0.039 | 0.081 |
| 2 | 38.600 | 85.200 |
| 3 | 7.060 | 16.300 |
| 4 | 0.870 | 0.770 |
| 5 | 2.090 | 4.310 |
| 7 | 2.730 | 5.580 |
| 8 | 0.150 | 0.490 |
| 9 | 0.180 | 0.410 |
| 10 | 0.160 | 0.360 |
| 11 | 5.080 | 5.890 |
| 12 | 0.400 | 1.080 |
| 13 | 13.200 | 9.460 |
| 14 | 2.180 | 3.900 |
| 15 | 0.220 | 0.480 |
| 16 | 19.200 | 54.400 |
| 17 | 34.200 | >100.000 |
| 18 | 0.090 | 0.220 |
| 19 | 0.060 | 0.110 |
| 20 | 0.009 | 0.140 |
| 21 | 0.039 | 0.061 |
| 22 | 0.049 | 0.100 |
| 23 | 0.030 | 0.100 |
| 24 | 0.022 | 0.065 |
| 25 | 0.068 | 0.150 |
| 26 | 2.556 | 2.027 |

### Example 28: Bioavailability Studies

Example 14 is a prodrug form of Example 1. Examples 19 to 22 and 24 to 26 are prodrugs of example 18. The following pharmacokinetic study was performed to demonstrate bioavailability of Example 18 from Example 19. Example 19 was formulated in 0.9% w/v saline pH 4 (adjusted with 1 M HCl) for IV dosing. The formulation was dosed at 2 mg/kg by femoral catheter to two male rats each (170-250 g). Jugular vein catheter serial blood samples were taken at 0.033, 0.083, 0.167, 0.5, 1, 2, 4, 8, and 24 hrs post-dose. For PO dosing, Example 19 was formulated in deionized water pH 4 (adjusted with 1 M HCl) and dosed at 5 mg/kg by oral gavage to two male rats each (170-250 g). Serial blood samples were taken by jugular vein catheter at 0.25, 0.5, 1, 1.5, 2, 3, 4, 8, and 24 hrs post dose. Plasma samples were generated from blood using low speed centrifugation. A single set of calibration standards containing Example 18 and Example 19 were prepared by spiking blank plasma. The samples and standards were extracted by precipitation with two volumes of acetonitrile followed by centrifugation. The results obtained were used to determine the Cl (mL/min/kg), Vdss (L/kg), Cmax (μM), AUC (μM h), tmax (h), and % F for both Example 18 and Example 19. Absolute bioavailability was determined by comparing the PO dose normalized AUC of Example 18 when dosed as Example 19, versus the dose normalized IV AUC of Example 18 when dosed as Example 18. Where appropriate, measured, not nominal, doses were used in the calculation. In an analogous fashion, the same procedure was repeated for Examples 14, 20 to 22, and 24 to 26. The results are shown in Tables 3 to 10. These results indicate that bioavailability may be increased by incorporating certain amino acid moieties as prodrugs.

TABLE 3

| | Example 19 | Example 18 |
|---|---|---|
| Cl (mL/min/kg) | 16.40 ^{#} | 7.30 * |
| Vdss (L/kg) | 0.47 ^{#} | 0.38 * |
| PO Cmax (μM) | 0.66 ^{#} | 4.40 ^{#} |
| PO AUC (μM.h) | 1.40 ^{#} | 15.6 ^{#} |
| Tmax (h) | 0.50 ^{#} | 1.50 ^{#} |
| %F | 8.30 ^{#} | 37.00 ^{#} |

^{#} observed value when dosed a pro-drug
* Observed value when dosed as payload.
NV No reportable value

TABLE 4

| | Example 20 | Example 18 |
|---|---|---|
| Cl (mL/min/kg) | 12.50 ^{#} | 7.30 * |
| Vdss (L/kg) | 0.21 ^{#} | 0.38 * |
| PO Cmax (μM) | 0.44 ^{#} | 8.10 ^{#} |
| PO AUC (μM.h) | 1.25 ^{#} | 30.90 ^{#} |
| Tmax (h) | 0.75 ^{#} | 1.25 ^{#} |
| %F | 5.10 ^{#} | 54.90 ^{#} |

^{#} observed value when dosed a pro-drug
* Observed value when dosed as payload.
NV No reportable value

TABLE 5

| | Example 21 | Example 18 |
|---|---|---|
| Cl (mL/min/kg) | 14.10 ^{#} | 7.30 * |
| Vdss (L/kg) | 0.22 ^{#} | 0.38 * |
| PO Cmax (μM) | 0.23 ^{#} | 10.20 ^{#} |
| PO AUC (μM.h) | 0.35 ^{#} | 32.40 ^{#} |
| Tmax (h) | 0.50 ^{#} | 1.25 ^{#} |
| %F | 1.50 ^{#} | 72.00 ^{#} |

^{#} observed value when dosed a pro-drug
* Observed value when dosed as payload.
NV No reportable value

TABLE 6

| | Example 22 | Example 18 |
|---|---|---|
| Cl (mL/min/kg) | 13.10 ^{#} | 7.30 * |
| Vdss (L/kg) | 0.20 ^{#} | 0.38 * |
| PO Cmax (μM) | 0.45 ^{#} | 4.70 ^{#} |
| PO AUC (μM.h) | 0.92 ^{#} | 16.00 ^{#} |
| Tmax (h) | 1.00 ^{#} | 1.75 ^{#} |
| %F | 4.50 ^{#} | 39.00 ^{#} |

^{#} observed value when dosed a pro-drug
* Observed value when dosed as payload.
NV No reportable value

TABLE 7

| | Example 24 | Example 18 |
|---|---|---|
| Cl (mL/min/kg) | 8.40 ^{#} | 7.30 * |
| Vdss (L/kg) | 0.20 ^{#} | 0.38 * |
| PO Cmax (μM) | 0.88 ^{#} | 1.30 ^{#} |
| PO AUC (μM.h) | 2.90 ^{#} | 6.40 ^{#} |
| Tmax (h) | 1.75 ^{#} | 2.50 ^{#} |
| %F | 6.40 ^{#} | 11.30 ^{#} |

^{#} observed value when dosed a pro-drug
* Observed value when dosed as payload.
NV No reportable value

TABLE 8

| | Example 26 | Example 18 |
|---|---|---|
| Cl (mL/min/kg) | 26.60 ^{#} | 7.30 * |
| Vdss (L/kg) | 0.15 ^{#} | 0.38 * |
| PO Cmax (μM) | NV ^{#} | 15.30 ^{#} |
| PO AUC (μM.h) | NV ^{#} | 37.30 ^{#} |
| Tmax (h) | NV ^{#} | 0.75 ^{#} |
| %F | NV ^{#} | 66.30 ^{#} |

^{#} observed value when dosed a pro-drug
* Observed value when dosed as payload.
NV No reportable value

TABLE 9

| | Example 25 | Example 18 |
|---|---|---|
| Cl (mL/min/kg) | 28.30 ^{#} | 7.30 * |
| Vdss (L/kg) | 0.18 ^{#} | 0.38 * |
| PO Cmax (μM) | 0.04 ^{#} | 8.83 ^{#} |
| PO AUC (μM.h) | NV ^{#} | 26.20 ^{#} |
| Tmax (h) | 0.25 ^{#} | 1.00 ^{#} |
| %F | NV ^{#} | 56.00 ^{#} |

^{#} observed value when dosed a pro-drug
* Observed value when dosed as payload.
NV No reportable value

TABLE 10

| | Example 14 | Example 1 |
|---|---|---|
| Cl (mL/min/kg) | 46.10 ^{#} | 8.26 * |
| Vdss (L/kg) | 0.47 ^{#} | 0.46 * |
| PO Cmax (μM) | 0.13 ^{#} | 3.42 ^{#} |
| PO AUC (μM.h) | NV ^{#} | 15.50 ^{#} |
| Tmax (h) | 0.25 ^{#} | 1.75 ^{#} |
| %F | NV ^{#} | 42.30 ^{#} |

^{#} observed value when dosed a pro-drug
* Observed value when dosed as payload.
NV No reportable value

1. A compound of formula (I), or a pharmaceutically acceptable salt thereof: 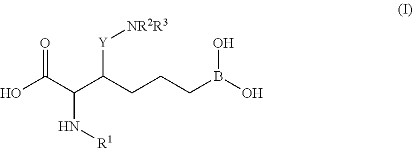
wherein
R¹ is selected from hydrogen, —CH₃ and —(C═O)CH(R^{1a})NH₂;
R^{1a} is C₁-C₄ alkyl;
Y is —(CH₂)ₙ— or —(C═O)—;
n is an integer selected from 1 and 2;
R² is selected from hydrogen, —CH₃ and —(C═X)R⁴ and R³ is hydrogen or —CH₃; or
R² and R³, together with the nitrogen to which they are attached, are linked to form a 6-membered heterocyclic ring;
X is NH or O;
R⁴ is —CH₃ or —[CH(R^{4a})]ₘNH₂;
m is an integer selected from 0 or 1; and
R^{4a} is hydrogen or C₁-C₆ alkyl.

2. The compound of claim 1, wherein the compound of formula (I), or a pharmaceutically acceptable salt thereof, is a compound of formula (Ia):

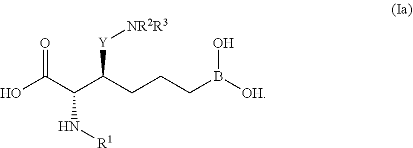

3. The compound of claim 1, wherein the compound of formula (I), or a pharmaceutically acceptable salt thereof, is a compound of formula (Ib):

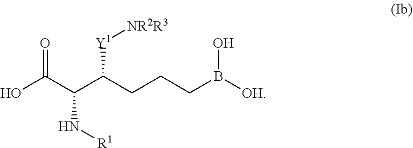

4. A compound of formula (II), or a pharmaceutically acceptable salt thereof: 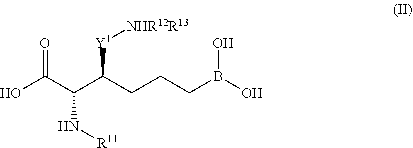
wherein
R¹¹ is selected from hydrogen, —CH₃ and 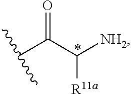
wherein * indicates (S) stereochemistry;
Y¹ is —(CH₂)ₚ— or —(C═O)—;
p is an integer selected from 1 and 2;
R^{11a} is C₁-C₄ alkyl;
R¹² is selected from hydrogen, —CH₃ and —(C═X¹)R¹⁴ and R¹³ is hydrogen or —CH₃; or
R¹² and R¹³, together with the nitrogen to which they are attached, are linked to form a 6-membered heterocyclic ring;
X¹ is NH or O;
R¹⁴ is —CH₃ or 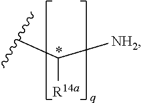
wherein * indicates (S) stereochemistry;
R^{14a} is C₁-C₄ alkyl; and
q is an integer selected from 0 and 1.

5. A compound of formula (III), or a pharmaceutically acceptable salt thereof: 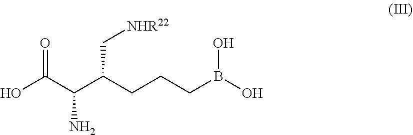
wherein
R²² is hydrogen or 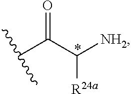
wherein * indicates (S) stereochemistry; and
R^{24a} is C₁-C₄ alkyl.

6. A compound of formula (IV), or a pharmaceutically acceptable salt thereof: 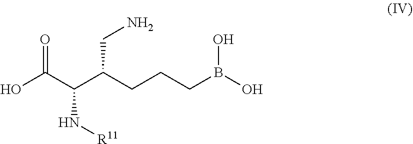
wherein
R¹¹ is selected from 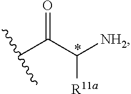
wherein * indicates (S) stereochemistry; and
R^{11a} is C₁-C₄ alkyl.

7. A compound of Table 1, or a pharmaceutically acceptable salt thereof.

8. A pharmaceutical composition comprising a compound of any one of claims 1 to 7, or a pharmaceutically acceptable salt thereof, and a pharmaceutically acceptable carrier, diluent or excipient.

9. A method of treating cancer comprising administering to a subject a therapeutically effective amount of a compound of any one of claims 1 to 7, or a pharmaceutically acceptable salt thereof.

10. A compound of any one of claims 1 to 7, or a pharmaceutically acceptable salt thereof, for treating cancer.

11. A pharmaceutical composition of claim 8 for treating cancer.

12. Use of a compound of any one of claims 1 to 7, or a pharmaceutically acceptable salt thereof, in the manufacture of a medicament for treating cancer.

13. A method of treating a respiratory inflammatory disease comprising administering to a subject a therapeutically effective amount of a compound of any one of claims 1 to 7, or a pharmaceutically acceptable salt thereof.

14. A compound of any one of claims 1 to 7, or a pharmaceutically acceptable salt thereof, for treating a respiratory inflammatory disease.

15. A pharmaceutical composition of claim 8 for treating a respiratory inflammatory disease.

16. Use of a compound of any one of claims 1 to 7, or a pharmaceutically acceptable salt thereof, in the manufacture of a medicament for treating a respiratory inflammatory disease.
